(19) United States
(12) Patent Application Publication
(10) Pub. No.: US 2025/0019769 A1
(43) Pub. Date: Jan. 16, 2025

(54) REPURPOSING DRUGS FOR NON-SMALL CELL LUNG CANCER

(71) Applicant: WEST VIRGINIA UNIVERSITY BOARD OF GOVERNORS ON BEHALF OF WEST VIRGINIA UNIVERSITY, Morgantown, WV (US)

(72) Inventor: Nancy Lan GUO, Morgantown, WV (US)

(21) Appl. No.: 18/684,274

(22) PCT Filed: Aug. 18, 2022
(86) PCT No.: PCT/US2022/075136

§ 371 Date: Feb. 16, 2024

Related U.S. Application Data
(60) Provisional application No. 63/355,587, filed on Jun. 25, 2022.
(60) Provisional application No. 63/355,353, filed on Jun. 24, 2022.
(60) Provisional application No. 63/344,200, filed on May 20, 2022.
(60) Provisional application No. 63/265,649, filed on Dec. 17, 2021.
(60) Provisional application No. 63/234,393, filed on Aug. 18, 2021.

(51) Int. Cl. C12Q 1/6886; A61K 31/404; G16H 50/20

(52) U.S. Cl. CPC C12Q 1/6886; A61K 31/404; G16H 50/20

(57) ABSTRACT

There are currently no effective biomarkers to select chemotherapy, immunotherapy, and radiotherapy for treating lung cancer patients. This study identified genetic networks containing major immune-checkpoint inhibitors CD27, PD1, and PDL1, and their associated prognostic genes and proliferation genes in lung cancer tumors. A co-expression network that was missing in tumor B cells was discovered that can promote lung cancer proliferation. A 5-gene and a 9-gene prognostic models were developed and validated in extensive cohorts to select patients at a high risk for developing metastasis. CRISPR-Cas9 and RNA interference screening data were used in the selection of proliferation genes. These genes were associated with chemoresponse and radio-therapy response in lung cancer cell lines and patient tumors. These immune-omics networks led to the discovery of repositioning drugs for improving lung cancer treatment.

### CROSS-REFERENCE TO RELATED APPLICATIONS

This application claims priority to, and the benefit of, U.S. Provisional Application No. 63/234,393, filed Aug. 18, 2021; U.S. Provisional Application No. 63/265,649, filed Dec. 17, 2021; U.S. Provisional Application No. 63/344,200, filed May 20, 2022; U.S. Provisional Application No. 63/355,353, filed Jun. 24, 2022; U.S. Provisional Application No. 63/355,587, filed Jun. 25, 2022; all of which are hereby incorporated by reference in their entireties.

### STATEMENT REGARDING FEDERALLY SPONSORED RESEARCH OR DEVELOPMENT

This invention was made with government support under Grant Nos. LM009500 and RR016440 awarded by the National Institutes of Health. The government has certain rights in the invention.

### BACKGROUND

Lung cancer remains the leading cause of cancer-related deaths in the world, and non-small cell lung cancer (NSCLC) accounts for almost 80% of lung cancer deaths. The heterogeneous nature of lung cancer makes it a very difficult disease to treat. According to the National Cancer Institute, 56% of lung cancer cases are at the distant stage, meaning that cancer has metastasized at the time of diagnosis. NSCLC accounts for 84% of lung cancer cases. Major histology of NSCLC includes lung adenocarcinoma (LUAD), squamous cell lung carcinoma (LUSC), and large cell carcinoma. Surgical resection is the major treatment for early-stage NSCLC. However, about 22-38% of stage I NSCLC patients will develop tumor recurrence within five years following the surgery. It is therefore preferable to select early-stage NSCLC patients for more aggressive treatment. NSCLC patients with stage II and above receive chemotherapy, with additional radiation for stage III and IV patients. While adjuvant chemotherapy of stage II and stage III disease has resulted in 10-15% increased overall survival, the prognosis for early-stage NSCLC remains poor, indicating that patients may not benefit from the prescribed chemotherapy. Currently, there are no clinically available biomarkers to predict the risk for tumor recurrence and the clinical benefits of specific chemotherapy in resectable NSCLC patients of all histological subtypes. Nor are there effective biomarkers to select chemotherapy, immunotherapy, and radiotherapy for treating lung cancer patients.

### SUMMARY

Aspects of the present disclosure are related to repurposing drugs for non-small cell lung cancer. In one aspect, among others, a method for treating non-small cell lung cancer (NSCLC) in a subject, comprises administering to the subject an effective amount of a compound selected from the group consisting of anisomycin, puromycin, aztreonam, ivermectin, natural compound piperlongumine, disulfiram (Antabuse), trichostatin A, dasatinib, lestaurtinib, homosalate, FK 888, penfluridol, and lestaurtinib in a pharmaceutically acceptable carrier.

In another aspect, a method for treating non-small cell lung cancer (NSCLC) in a subject comprises assaying a sample from the subject for expression levels of COPA, CSE1L, EIF2AK3, FOSL1, LSM3, MCM5, PMPCB, POLR1B, POLR2F, PSMD11, RPL32, RPS18, SNRPE, SPP1, IFITM3, ATP1B4, PRRX1, FUT7, POF1B, ZNF671, ZBED2, C10orf10, ERH, KLHL6, SERPING1, TRIM29, CCR2, TNFSF12-TNFSF13, ATP1B4, PRDM1, RGS17, CD80, ARK1C1, DEFB103A, CSPP1, AKR1C2, DSG3, PRDM1, ABCA13, ARSE, PTAFR, ZEB1, ERH, F13A1, KLHL6, PRRX1, GPX2, LAIR1, TRIM22, PTAFR, RAB25, ESRP1, SERPINB5, KLHL6, CD80, SH2D1A, CD27, TGM5, CD274, HLA-F, LY75, F13A1, CTSH, CPNE8, ARSE, HLA-DMB, SLC39A8, HNMT, CTSH, CDH3, HNMT, ABCA13, F13A1, DUSP2, HLA-DRB1, TIGIT, TNIP3, ANAPC5, OAS1, VCP, IL2RA, LMNA, PPP2R1A, TNIP3, CCND2, TSC22D3, HRH1, CCT3, EWSR1, TUBA1B, HSD17B13, MAP4, HRH1, CD74, HLA-DRA, HSD17B13, EXOC4, GBP4, TSC22D3, or a combination thereof; and, detecting elevated levels of DUSP2, VCP, IL2RA, LMNA, CCT3, PPP2R1A, POF1B, CD274, HLA-F, LY75, PRRX1, and/or F13A1, detecting reduced levels of PSMD11, ANAPC5, OAS1, FUT7, ZNF671, ZBED2, C10orf10, CTSH, and/or TRIM29, or a combination thereof, in a sample from the subject, and treating the subject with cisplatin; detecting elevated levels of RPS18, PMPCB, TNIP3, IL2RA, CCND2, GBP4, ERH, KLHL6, AKR1C1, AKR1C2, SERPINB5, and/or POLR1B, detecting reduced levels of IFITM3, C10orf10, SERING1, PSMD11, LMNA, PPP2R1A, TUBA1B, CPNE8, and/or EIF2AK3, or a combination thereof, and treating the subject with docetaxel; detecting elevated levels of RPL32, TSC22D3, CCT3, VCP, KLHL6, TRIM29, IFITM3, SERPINB5, PRDM1, RGS17, CD80, AKR1C1, AKR1C2, DEFB103A, CSPP1, ARSE, HLA-DMB, TRIM22, POF1B, LSM3, and/or POLR2F, detecting reduced levels of CSE1L, DUSP2, HRH1, LMNA, LY75, TGM5, F13A1, IFITM3, CCR2, TNFSF12-TNFSF13, ATP1B4, SERPING1, DSG3, and/or FOSL1, or a combination thereof, and treating the subject with erlotinib; detecting elevated levels of CCT3, EWSR1, TUBA1B, VCP, HSD17B13, LMNA, PSMD11, PRDM1, ATP1B4, SERPING1, ABCA13, HLA-F, ABCA13, and/or SERPINB5, detecting reduced levels of TGM5, TRIM29, and/or ARSE, or a combination thereof, and treating the subject with etoposide; detecting elevated levels of CCND2, CCT3, CCT3, HLA-DRA, TSC22D3, CCT3, DEFB103A, PTAFR, CSPP1, KLHL6, TRIM29, and/or SLC39A8, detecting reduced levels of COPA, OAS1, VCP, HSD17B13, LMNA, MAP4, TUBA1B, HRH1, HSD17B13, ZEB1, ERH, F13A1, RGS17, ATP1B4, PRRX1, SERPING1, and/or ARSE, or a combination thereof, and treating the subject with gefitinib; detecting elevated levels of CCT3, CD74, DUSP2, HLA-DRA, TIGIT, LMNA, VCP, ARSE, GPX2, RGS17, LAIR1, TRIM22, TGM5, CTSH, HLA-F, SERPINB5, CDH3, HNMT, SPP1, and/or MCM5, detecting reduced levels of PSMC3, EXOC4, SERPING1, ARSE, C10orf10, ABCA13, AKR1C2, GPX2, C10orf10, and/or RPS18, or a combination thereof, and treating the subject with gemcitabine; detecting elevated levels of DUSP2, VCP, OAS1, TNIP3, PTAFR, TRIM29, RAB25, ESRP1, SERPINB5, KLHL6, CD80, CD27, CDH3, PRRX1, PMPCB, RPS18, POLR1, and/or LY75, detecting reduced levels of PSMD11, PPP2R1A, HSD17B13, CD74, HLA-DRA, TUBA1B, SH2D1A, C10orf10, HLA-F, SLC39A8, CTSH, and/or TRIM29, or a combination thereof, and treating the subject with paclitaxel; detecting elevated levels of CCT3, DUSP2, ANAPC5, HRH1, and/or RPS18, detecting reduced levels of SH2D1A, or a combination thereof, and treating the subject with pemetrexed; detecting elevated levels of FUT, VCP, CCT3, DUSP2, CCND2,, SERPINB5, AKR1C2, AKR1C1, F13A1, HLA-F, and/or POLR2F, detecting reduced levels of CSE1L, EXOC4, HRH1, LMNA, TNFSF12-TNFSF13, DSG3, SNRPE, and/or CTSH, or a combination thereof, and treating the subject with vinorelbine; or detecting elevated levels of DUSP2, TIGIT, TGM5, and/or IFITM3, detecting reduced levels of HLA-DRB1, TNIP3, ATP1B4, and/or PRRX1, or a combination thereof, and treating the subject with carboplatin.

In another aspect, a system comprises a computing device comprising a processor and memory; and an application to generate Boolean implication networks when compiled with the computing device, the application comprising machine readable instructions stored in the memory that, when executed by the processor, cause the computing device to at least: generate one or more multi-omics networks from patient cohort information based upon one or more Boolean implication networks; identify prognostic genes based upon the one or more multi-omics networks and patient gene expression profiles and associated clinical information; identify proliferation genes based upon the one or more multi-omics networks and screening results in cell lines; delineating a final multi-omics network based upon identified prognostic and proliferation genes; and identifying a repurposing drug based upon the final multi-omics network.

In various aspects, the screening results can comprise CRSPR-Cas9 and RNA interference (RNAi). The repurposing drug can be identified based upon the final multi-omics network using a Connectivity Map database. The patient cohort information can be associated with cancer, and the patient cohort information can include disease-free survival time with status and/or disease-specific survival time with status. The cancer can be non-small cell lung cancer (NSCLC) and/or the cell lines can be NSCLC.

In various aspects, the method can comprise obtaining the patient cohort information from a patient cohort database. The patient cohort information can include genome-scale mRNA expression profiles and DNA copy number variations from the patient cohort database. The patient cohort information can be obtained from a NSCLC patient cohort. The patient cohort information can be evaluated based upon a plurality of implication rules of the one or more Boolean implication networks. In various aspects, the method can comprise identifying genes associated with radiotherapy and chemotherapy.

In another aspect, a kit to predict the risk of NSCLC tumor recurrence or metastasis comprising primers and/or probes is configured to detect EIF2AK3, F2RL3, FOSL1, SLC25A26, and SPP1; or configured to detect ANAPC5, CCT3, EWSR1, EXOC4, HRH1, MAP4, PPP2R1A, TUBA1B, and VCP.

In another aspect, a method to predict the risk of NSCLC tumor recurrence or metastasis in a subject comprises assaying a sample from the subject for synthesized cDNA levels representing mRNA levels of one or more of EIF2AK3, F2RL3, FOSL1, SLC25A26, and SPP1, and generating a risk score from the mRNA levels; or assaying a sample from the subject for synthesized cDNA levels representing mRNA levels of one or more of ANAPC5, CCT3, EWSR1, EXOC4, HRH1, MAP4, PPP2R1A, TUBA1B, and VCP, and generating a risk score from the mRNA levels. In various aspects, the method can comprise assaying a sample from the subject for synthesized cDNA levels representing mRNA levels of EIF2AK3, F2RL3, FOSL1, SLC25A26, SPP1; F2RL3, FOSL1, SLC25A26, SPP1; EIF2AK3, FOSL1, SLC25A26, SPP1; EIF2AK3, F2RL3, SLC25A26, SPP1; EIF2AK3, F2RL3, FOSL1, SPP1; EIF2AK3, F2RL3, FOSL1, SLC25A26; EIF2AK3, FOSL1, SLC25A2; EIF2AK3, FOSL1, SPP1; EIF2AK3, SLC25A26, SPP1; F2RL3, FOSL1, SLC25A26; F2RL3, SLC25A26, SPP1; FOSL1, SLC25A26, SPP1; EIF2AK3, FOSL1; EIF2AK3, SLC25A26; EIF2AK3, SPP1; F2RL3, SLC25A26; F2RL3, SPP1; FOSL1, SLC25A26; FOSL1, SPP1; or FOSL1. In various aspects, the method can comprise assaying a sample from the subject for synthesized cDNA levels representing mRNA levels of ANAPC5, CCT3, EWSR1, PPP2R1A, TUBA1B, VCP, EXOC4, HRH1, MAP4; ANAPC5, CCT3, EWSR1, TUBA1B, VCP, EXOC4, HRH1, MAP4; ANAPC5, CCT3, EWSR1, TUBA1B, EXOC4, HRH1, MAP4; ANAPC5, CCT3, EWSR1, TUBA1B, HRH1, MAP4; ANAPC5, CCT3, TUBA1B, HRH1, MAP4; ANAPC5, CCT3, EWSR1, TUBA1B, MAP4; ANAPC5, CCT3, TUBA1B; or HRH1.

In another aspect, a method for treating non-small cell lung cancer (NSCLC) in a subject, comprises assaying a sample from the subject for expression levels of one or more of CD27, MYC, SERPINB5, TTC16, VCP, and URM1; detecting reduced levels of one or more of CD27, TTC16, VCP, and URM1 and elevated levels of one or more of MYC and SERPINB5 compared to control values; and treating the subject with radiotherapy.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an example of a scheme to generate and utilize immune-omics networks, in accordance with various embodiments of the present disclosure.

FIGS. 2A-2C illustrate examples of CD27 multi-omics networks in non-small cell lung cancer (NSCLC), in accordance with various embodiments of the present disclosure.

FIG. 3 illustrates an example of experimentally confirmed interactions with CD27 retrieved from ingenuity pathways analysis (IPA), in accordance with various embodiments of the present disclosure.

FIGS. 4A-4C illustrate examples of CD27, PD1 and PDL1 immune-omics network in NSCLC, in accordance with various embodiments of the present disclosure.

FIGS. 5A-5C illustrate examples of a 5-gene prognostic model for NSCLC, in accordance with various embodiments of the present disclosure.

FIGS. 6A and 6B illustrate an example of the discovery of repurposing drug candidates, in accordance with various embodiments of the present disclosure.

FIG. 7 illustrates examples of compounds with a significantly negative correlation of drug concentration and the mRNA expression of CD27, PD1, or PDL1 in the CCLE NSCLC cell lines, in accordance with various embodiments of the present disclosure.

FIGS. 8A-D illustrate examples of a 7-gene signature and associated multi-omics network, in accordance with various embodiments of the present disclosure.

FIGS. 9A-D illustrate examples of an association between immune infiltration and expression of the multi-omics network genes, in accordance with various embodiments of the present disclosure.

FIGS. 10A-C illustrate examples of a correlation between mRNA and protein expression of genes in the multi-omics network, in accordance with various embodiments of the present disclosure.

FIG. 11 illustrates an example of a correlation of expression of ZNF71 with EMT markers in five NSCLC cell lines, in accordance with various embodiments of the present disclosure.

FIGS. 12A-F illustrate examples of ZNF71 gene level, ZNF71 KRAB, and ZNF71 KRAB-less co-expression networks in patient cohort GSE81089, in accordance with various embodiments of the present disclosure.

FIGS. 13A-C illustrate examples of dentification of functional pathways associated with the 7-gene multi-omics network and discovery of repostitioning drugs, in accordance with various embodiments of the present disclosure.

FIGS. 14A-F illustrate examples of identified proliferation and prognostic gene co-expression network in NSCLC B cells, in accordance with various embodiments of the present disclosure.

FIGS. 15A-B illustrate examples of differential expression patterns of selected genes in NSCLC T cells and B cells, in accordance with various embodiments of the present disclosure.

FIGS. 16A-B illustrate examples of differentially expressed genes in NSCLC T cells with prognostic indications, in accordance with various embodiments of the present disclosure.

FIG. 17 illustrates an example of an mRNA expression of VCP was associated with resistance to radiotherapy, in accordance with various embodiments of the present disclosure.

FIGS. 18A-C illustrate examples of discovery of repositioning drugs and functional pathways based on selected genes, in accordance with various embodiments of the present disclosure.

FIG. 19 illustrates an example of a schematic block diagram of a computing device 800 that can be utilized to execute an immune-omics network application for biomarker identification and repurposing drug discovery for evaluation and treatment of cancer such as NSCLC, in accordance with various embodiments of the present disclosure.

### DETAILED DESCRIPTION

Disclosed herein are various examples related to biomarker identification and repurposing drug discovery. To date, there are no prognostic/predictive biomarkers to select chemotherapy, immunotherapy, and radiotherapy in individual non-small cell lung cancer (NSCLC) patients. Major immune-checkpoint inhibitors (ICIs) have more DNA copy number variations (CNV) than mutations in The Cancer Genome Atlas (TCGA) NSCLC tumors. Nevertheless, CNV mediated dysregulated gene expression in NSCLC is not well understood. Genetic networks containing major immune-checkpoint inhibitors (ICSs) CD27, PD1, and PDL1, and their associated prognostic genes proliferation genes in lung cancer tumors can be identified. A prognostic model (e.g., a 5-gene prognostic model) can be developed and validated in extensive cohorts to select patients at a high risk for developing metastasis. CRISPR-Cas9 and RNA interference screening data can be used in the selection of proliferation genes. These genes can be associated with chemoresponse and radiotherapy response in lung cancer cell lines and patient tumors. The immune-omics network can lead to the discovery of repurposing drugs for improving lung cancer treatment. Reference will now be made in detail to the description of the embodiments as illustrated in the drawings, wherein like reference numbers indicate like parts throughout the several views.

Immunotherapy has rapidly gained attention of oncologists as an effective and less toxic treatment than chemotherapy in patients with advanced lung cancers. A recent study used paired single cell analysis to compare normal lung tissue and blood with tumor tissue in stage I NSCLC and found that early-stage tumors had already begun to alter the immune cells in their microenvironment. These results showed that immunotherapy could potentially be used to treat early-stage lung cancer patients. More recently, studies showed that immune-checkpoint inhibitors (ICIs) in combination with chemotherapy could improve NSCLC patient survival regardless of PDL1 expression. PDL1 and tumor mutational burdens have not been proven to be indicative biomarkers. To date, predictive biomarkers of immunotherapy are not well established except for PD1 or PDL1, and it is unlikely that a single marker is sufficient.

A 7-gene NSCLC prognostic and chemo-predictive qRT-PCR assay, including CD27, has been identified. The 7-gene NSCLC prognostic and predictive assay is disclosed in U.S. patent application Ser. No. 17/251,359 and International Patent Application No. PCT/US20/23597, published as WO 2019/241508 on Dec. 19, 2019, both of which are hereby incorporated by reference in their entireties. The protein expression of CD27 quantified with ELISA had a strong correlation with its mRNA expression in NSCLC tumors. CD27 is a new generation of ICI, which has shown promising results as adjuvant therapy in phase I/II clinical trials for multiple tumor types. Increasing evidence supports that CD27 agonist antibodies, either alone or combined with PD1-blockade, can improve the therapeutic efficacy of cancer vaccines and immunotherapy in general. Anti-CD27/CD20 showed therapeutic potential in recruiting and activating myeloid cells for enhanced killing of tumors. A review of The Cancer Genome Atlas (TCGA) data showed that CD27, PD1, PDL1, and CD20 had a higher frequency of DNA copy number variations (CNV) than mutations in LUAD and LUSC tumors. Nevertheless, CNV mediated dysregulated gene expression in NSCLC is not well understood. FoundationOneDx assay is an FDA-approved companion test for tumor mutation burdens in stage IV patients. Currently, there are no effective CNV or mRNA expression-based biomarkers for predicting the risk of recurrence/metastasis in stage I to III patients, or for the selection of immunotherapy, chemotherapy, and radiotherapy in individual NSCLC patients in all cancer stages.

The tumor immune microenvironment is a complex network of genes and proteins functioning in immune and stromal cells as well as other systemic host factors, which together determine the immunological status of the host-tumor interactions. Given this multidimensionality, a gene signature that integrates these elements should be developed to select patients for personalized immunotherapy, in combination with chemotherapy and/or radiotherapy. Recent studies systematically revealed genomic instability and transcriptomic dysregulation in tumor immune microenvironment. Innovative network approaches are needed to identify interweaving molecular interactions with implications in prognosis, proliferation, and response to chemotherapy, immunotherapy, and radiotherapy for improving cancer treatment. Such pathway and network approaches can lead to the discovery of novel therapeutic targets as well as repositioning drug candidates.

In the current disclosure, a novel Boolean implication network scheme is disclosed that models CNV and gene expression (GE) profiles in NSCLC tumors (n=371) to decipher molecular networks for CD27, and dissect those involving CD27, PD1, and PDL1. Such revelation can provide insights into molecular mechanisms underlying immunotherapy targeting CD27 alone, or in combination with PD1/PDL1-blockade. Significantly enriched cytobands, pathways and gene families can be analyzed for the constructed CD27 networks. Based on the identified multi-omics network of CD27, PD1, and PDL1, proliferation genes can be identified from CRISPR-Cas9 (n=78) and RNAi (n=92) screening data; prognostic genes can be selected and validated in NSCLC tumors (n=1,163). The identified genes can be examined for their association with chemotherapy in the Cancer Cell Line Encyclopedia (CCLE) NSCLC panel (n=117) and radiotherapy in TCGA patient tumors (n=966). The immune infiltration associated with the identified genes can be investigated in myeloid dendritic cells, macrophages, neutrophils, CD4+ T cells, CD8+ T cells, and B cells. Further, repurposing drug candidates for NSCLC treatment can be discovered based on the identified immune-omics network.

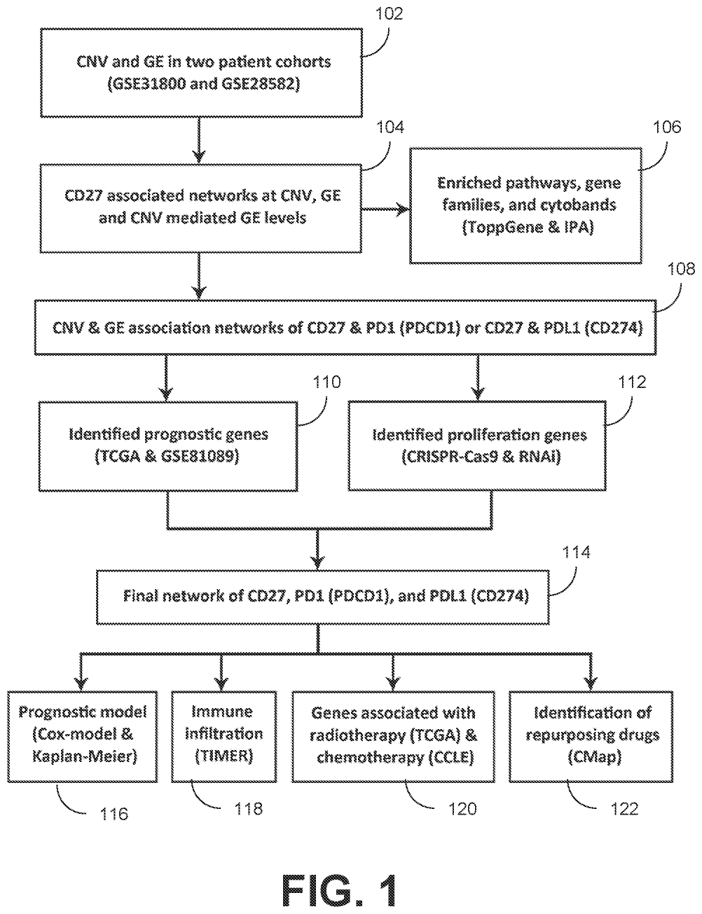

FIG. 1 illustrates an example of an overall scheme that can result in biomarker identification and repurposing drug discovery that at least portions of which may be implemented as, e.g., an immune-omics network application. Beginning at 102, patient cohorts can be obtained. Associated multi-omics networks can be identified at 104 using preprocessing and/or an implication network methodology and functional enrichment analysis can be carried out at 106 to generate multi-omics networks at 108. Prognostic genes and proliferation genes can be identified at 110 and 112, with the delineation of the final multi-omics network being provided at 114. With the final multi-omics network, a prognostic model can be developed at 116 and correlation of immune infiltration can be estimated at 118. Genes associated with radiotherapy and chemotherapy can be identified at 120 and repurposing drug candidates discovered at 122.

### Materials and Methods

### Patient Cohorts

NSCLC patient cohort GSE31800. DNA copy number profiles of 271 NSCLC tumor samples have been measured, including 179 adenocarcinomas and 92 squamous cell carcinomas (raw data available at NCBI GEO with the accession number GSE31800). The DNA copy number profiles were measured with whole genome tiling path array CGH. A total of 49 out of the 271 samples had matched custom microarray gene expression profiles, with 29 adenocarcinomas and 20 squamous cell carcinomas.

NSCLC patient cohort GSE28582. A publicly available NSCLC patient cohort (n=100) was included in this study, including 50 adenocarcinomas, 22 large cell carcinomas, and 28 squamous cell carcinomas (raw data available at NCBI GEO with the accession number GSE28582). All the samples had matched DNA copy number variation profiled by SNP array and microarray gene expression data. Patients who survived shorter than 20 months after treatment were defined as short-term survivors, and those who survived longer than 58 months after treatment were defined as long-term survivors.

NSCLC patient cohort GSE81089. A total of 199 NSCLC patient tumors were collected. Deep RNA sequencing of the patient tumors was generated with Illumina HiSeq 2500 (raw data available at NCBI GEO with the accession number GSE81089). Patients with sufficient survival information (n=197) were included in this study. The raw RNA-seq data were processed with Salmon to quantify the expression of ZNF71 at the isoform level by using transcripts per million (TPM) reads

TCG. A NSCLC patient cohorts. The TCGA-LUAD (n=515) and TCGA-LUSC (n=501) data was available from LinkedOmics (http://linkedomics.org/, accessed on 28 Apr. 2021). The gene expression was quantified with Illumina HiSeq platform. The RNA-Seq data values were log-transformed. The TCGA-LUSC and TCGA-LUAD dataset also had the cancer stage and radiotherapy information for each sample.

### Data Pre-Processing

CNV data pre-processing. The original copy number data of GSE31800 was converted from human genome assembly Hg18 to Hg38 in this study. According to the converted chromosomal locations, 26,061 genes were matched. Bioconductor R package “CGHbase” (v1.46.0) and “CGHcall” (v2.48.0) were used to call the gains and losses of the copy number data.

The original GSE28582 data was processed by converting the Hg17 reference to Hg38 in this study. According to the converted chromosomal locations, 12,842 genes were matched. PennCNV package was used to process the SNP array data. The CNV data was categorized into 3 levels: 1 (amplification), −1 (deletion), and 0 (normal).

Gene expression data pre-processing. To define the level of gene expression, 27 housekeeping genes (ACTB, B2M, CDKN1B, ESD, FLOT2, GAPDH, GRB2, GUSB, HMBS, HPRT1, HSP90AB1, IPO8, LDHA, NONO, PGK1, POLR2A, PPIA, PPIH, PPP1CA, RHOA, RPL13A, SDCBP, TBP, TFRC, UBC, YAP1, YWHAZ) were used to define the thresholds of gene expression categorization. The total percentage of over-expression and under-expression samples for all the housekeeping genes was fixed to be 30%, and the number n which meets the following equation (1) was computed for each dene:

$$( the number of samples with expression > mean + n \times std ) + ( the number of samples with expression < mean - n \times std ) = 30 \% of the total number of samples , \qquad ( 1 )$$

where std is the standard deviation.

The averaged n value of all 27 housekeeping genes was 0.874 in the dataset GSE31800 and 0.977 in the dataset GSE28582. These n values were used to categorize each gene expression dataset. The mean and standard deviation for each gene were calculated. The sample with a gene expression higher than (mean+n×std) was defined as over-expressed; the sample with a gene expression lower than (mean−n×std) was defined as under-expressed; the sample with a gene expression in between the two thresholds was defined as normal. Other thresholds may be defined for use. After categorizing with the above-mentioned thresholds, more than 50% of the genes in both patient cohorts had more over-expressed or under-expressed samples than the housekeeping genes.

### Boolean Implication Networks

The disclosed Boolean implication network algorithm was based on prediction logic. Formal logic rules relating two binary variables were inferenced for each pair of genes to form the implication network. The algorithm can be improved by counting the error cell of each implication rule such that it can model not only the binary variable but also multivariate data. This network induction algorithm is generic for various general applications, including multinomial datasets and multi-classification problems.

The Boolean implication algorithm evaluates six implication rules: A→B (positive implication), A→¬B (forward negative implication), ¬A→B (inverse negative implication), ¬A→¬B (negative implication), A [symbol] B (positive equivalence), and A [symbol] ¬B (negative equivalence). (A &¬B) is the error cell for A→B. (A & B) is the error cell for A→¬B. (¬A &¬B) is the error cell for ¬A→B. (¬A & B) is the error cell for ¬A→¬B. (A &¬B) and (¬A & B) are the error cells for A [symbol] B. (A & B) and (¬A &¬B) are the error cells for A [symbol] ¬B.

The Boolean implication algorithm can rely predominantly upon error cells for a particular rule while calculating the scope (Uₚ), indicating how much the data is covered, and precision (∇ₚ), indicating accuracy, for that rule. For instance, while looking for co-amplification in a pair of genes (A→B), the algorithm checks if a gene is amplified, and the remaining two states of deletion and normalcy become simply the negate of amplification; the error would be when gene A is amplified but gene B is not amplified. In this manner, the algorithm can use the same fundamental principle to create implication relations amongst genes, irrespective of the number of states for each gene.

For each implication rule to be significant, it is identified as having a scope and precision value which is greater than the threshold value, and the greatest amongst all the rules for the pair of variables in question. The threshold for scope and precision can depend on the significance level in statistical tests. An example of the algorithm can be described as:

```
Begin
  Set a significance level ∇ₘᵢₙ and a minimal Uₘᵢₙ
  For nodeᵢ, i ∈ [0, νₘₐₓ − 1] and nodeⱼ, j ∈ [i +1, νₘₐₓ]
      (Note: νₘₐₓ is the total number of nodes)
    For all empirical case samples N, compute a contingency table:
```

| | |
|---|---|
| Mᵢⱼ = | N₁₁ N₁₂ |
| | N₂₁ N₂₂ |

For each relation type k out of the six cases find the solution:

| | |
|---|---|
| | Max Uₚ |
| Subject to | Max Uₚ > Uₘᵢₙ |
| | ∇ₚ > ∇ₘᵢₙ |
| | ∇_{error cells} > ∇_{non-error cells} |

```
    If the solution exists, then return a type k relation
  End
```

In the contingency table Mᵢⱼ, i stands for the index of the first gene (A) in the methodology and j stands for the index of the second gene (B). One association type (i.e., co-amplification, etc.) was tested each time. N₁₁ represents the number of samples where both A and B have a true statement in the examined association type; N₁₂ is the count of samples where A is true, but B is not true; N₂₁ is the count of samples where A is not true, but B is true; and N₂₂ is the number of samples where both A and B are not true. For a single error cell, where Nᵢⱼ is the number of error occurrences, N_{i⋅} is the row sum of contingency table Mᵢⱼ, N_{⋅j} is the column sum of contingency table Mᵢⱼ. N is the total number, which is the sum of N₁₁, N₁₂, N₂₁, and N₂₂. The scope Uₚ and the precision ∇ₚ can be defined as the equations (2):

$${U}_{p} = {U}_{ij} = \frac{{N}_{i .} \times {N}_{. j}}{{N}^{2}} , {∇}_{p} = {∇}_{ij} = 1 - \frac{{N}_{ij}}{N \times {U}_{p}} . \qquad ( 2 )$$

For multiple error cell types, the scope Uₚ and the precision ∇ₚ can be defined as the equations (3), where ωᵢⱼ=1 for error cells; otherwise, ωᵢⱼ=1.

$${U}_{p} = {\sum }_{i} {\sum }_{j} {ω}_{ij} \times {U}_{ij} , {∇}_{p} = {\sum }_{i} {\sum }_{j} ( \frac{{ω}_{ij} \times {U}_{ij}}{{U}_{p}} ) {∇}_{ij} . \qquad ( 3 )$$

The pairwise gene relationships at the CNV level included co-amplification, co-deletion, amplification-deletion, and deletion-amplification. The pairwise gene relationships at gene expression level included co-upregulation, co-downregulation, upregulation-downregulation, and downregulation-upregulation. The modelled CNV and gene expression cross-level included amplification-upregulation, amplification-downregulation, deletion-upregulation, and deletion-downregulation. The scope and precision threshold can be adjusted by altering the statistical significance for the implication rules in the implementation. The threshold value was calculated from a one-tailed z-test based on the sample size by setting the ideal z value. The z value used for this study was 1.64 (95% confidence interval, α=0.05, one-tailed z-tests).

### Functional Enrichment Analysis Using ToppGene

Functional enrichment analysis can be carried out. ToppFun of the ToppGene suite was used to detect functional enrichment including pathways, gene families, and cytobands. The ToppFun tool used the false discovery rate (FDR) multiple correction method with a significance cutoff level of 0.05 in the enrichment analysis.

### Ingenuity Pathways Analysis

Ingenuity pathways can be analyzed. Functional involvement of genes in cancer, immunological disease, and inflammatory disease was examined with Qiagen Ingenuity Pathways Analysis (IPA) (Ingenuity® Systems, Qiagen, Hilden, Germany). IPA is a functional pathway analysis tool incorporating genes, cellular species such as proteins, and chemical compounds with information curated from published literature.

### Cancer Cell Line Encyclopedia (CCLE)

Gene expression data for CCLE were downloaded from DepMap 20Q2 (https://figshare.com/articles/dataset/DepMap_20Q2_Public/12280541, accessed on 1 Apr. 2021). Gene expression data were obtained from the CCLE data portal (https://data.broadinstitute.org/ccle/CCLE_RNAseq_081117.rpkm.gct, accessed on 1 Apr. 2021). RNA-seq data were quantified using the GTEx pipelines. A total of 117 NSCLC cell lines were included in this analysis.

Proteomics data for CCLE were downloaded from the Gygi lab (https://gygi.hms.harvard.edu/publications/ccle.html, accessed on 1 Oct. 2021). A total of 63 NSCLC cell lines were included in this analysis.

### CRISPR-Cas9 Assays

Gene knockout effects in CCLE using CRISPR-Cas9 screens were quantified in Project Achilles. The data were obtained from DepMap 20Q2 (https://figshare.com/articles/dataset/DepMap_20Q2_Public/12280541, accessed on 1 Apr. 2021). The CRISPR-Cas9 data were processed with the CERES method. Gene effects in each cell line were normalized such that the median non-essential gene knockout effect is 0 and the median essential gene knockout effect is −1. A gene is defined as an essential gene if it is essential to the cell growth in each line; otherwise, it is defined as a non-essential gene. There were 78 NSCLC cell lines with genome-scale CRISPR-Cas9 knockout results. A normalized dependence score less than −0.5 indicates a significant effect in CRISPR-Cas9 knockout.

### RNAi Functional Assays

Genome-scale RNAi screening data in CCLE were obtained from Project Achilles (https://depmap.org/R2-D2/, accessed on 1 Apr. 2021). The DEMETER2 method was used to estimate average gene dependency scores in each cell line for short hairpin RNA (shRNA) libraries. Gene dependency scores were standardized with DEMETER2 such that the median of the across-cell-line average dependency scores of the positive control gene set was −1 and that of the negative control gene set was 0. There were 92 NSCLC cell lines with genome-scale RNAi screening results normalized with DEMTER2. A normalized dependence score less than −0.5 indicates a significant effect in RNAi knockdown.

### Immune Infiltration Estimation

TIMER 2.0 was used to find the association of gene expression and immune infiltration. TIMER is a comprehensive resource for systematically analyzing the immune infiltration of different cancer types (http://timer.cistrome.org/, accessed on 14 Jun. 2021). It provides the abundance of immune infiltration estimated by a variety of immune deconvolution methods.

### PRISM Drug Response in CCLE

The growth inhibitory activity of 4,518 drugs was quantified in 578 human cancer cell lines using the PRISM molecular barcoding and multiplexed screening method. The PRISM dataset is available at the Cancer Dependency Map portal (https://depmap.org/portal/download/, accessed on 1 Apr. 2021). Drug responses of 9 commonly used chemotherapeutic regimens in treating NSCLC were included: carboplatin, cisplatin, paclitaxel, docetaxel, gemcitabine, vinorelbine, etoposide, gefitinib, and erlotinib. For each drug, cell lines with IC₅₀ or EC₅₀ value higher than the maximum dose were defined as resistant; cell lines with IC₅₀ or EC₅₀ value lower than the minimum dose were defined as sensitive. The remaining cell lines were divided into groups of resistant, sensitive, or partial response by using the mean±0.5 standard deviation (SD) of the drug activity metrics, including IC₅₀, ln(IC₅₀), EC₅₀, or ln(EC₅₀) values. Cell lines with a drug activity value less than the mean−0.5 SD were defined as sensitive to the drug, and those with a drug activity value between the mean+0.5 SD and the mean−0.5 SD were defined as having a partial response to the drug. Cell lines with a drug activity value greater than the mean+0.5 SD were defined as resistant to the drug. This categorization corresponds to the RECIST 1.1 system (i.e., complete response, partial response, and stable disease/disease progression) in evaluating chemotherapeutic response in solid tumors.

### Genomics of Drug Sensitivity in Cancer (GDSC1/2)

Drug screening data was downloaded from Genomics of Drug Sensitivity in Cancer (GDSC) Project (https://www.cancerrxgene.org/downloads/bulk_download, accessed on 15 Apr. 2021). The GDSC Project screened more than 1,000 genetically characterized human cancer cell lines with a wide range of anti-cancer therapeutic agents. Among the commonly used chemotherapeutic regimens in treating NSCLC drugs, 9 were found in GDSC1, including cisplatin, docetaxel, erlotinib, etoposide, gefitinib, gemcitabine, pemetrexed, and vinorelbine; 7 were found in GDSC2, including cisplatin, docetaxel, erlotinib, gefitinib, gemcitabine, paclitaxel, and vinorelbine. For each drug, cell lines were defined as resistant, sensitive, or partial response by using the mean±0.5 SD of the IC₅₀ values as described above.

### Drug Repurposing Using Cconnectivity Map (CMap)

CMap (https://clue.io/, accessed on 1 Oct. 2021)) was used to identify candidate small molecules as repurposing drugs based on the gene expression signatures. CMap is a database of gene expression profiles of pharmacologically perturbed cell lines. It is a resource of gene expression profiles of pharmacologic perturbed cell lines, using transcriptional expression signatures to connect disease, cell physiology, and therapeutics. It allows the use of gene expression signatures to connect small molecules, genes, and disease. CMap was used to find perturbagen signatures that connected with the gene expression signatures.

### Western Blots

NSCLC cell lines H441, H23 and H1299 (kind gift of Dr. Scott Weed, WVU), A549 and H1975 (kind gift of Dr. Ivan Martinez, WVU) were grown in DMEM (Corning, cat. #15-018-CV) supplemented with 10% FBS (HyClone, UT, USA), 2 mM L-glutamine (Corning, NY, USA; cat. #25-005-CI), and 1×Antibiotic Antimycotic Solution (Corning, NY, USA; cat. #30-004-CI). All cells were maintained at 37° C. in a 5% CO₂ incubator. Whole/total cell lysates were prepared in nonreducing Laemmli buffer as described in. Nuclear-cytoplasmic fractionation was performed using ProteoJET Cytoplasmic and Nuclear Protein Extraction Kit (Fermentas, cat. #K0311) as described in.

Protein concentration was quantified by Pierce BCA Protein Assay (ThermoFisher, MA, USA; cat #23225). Lysates with equal amount of total protein were separated on 4% to 12% Bis-Tris NuPAGE Novex gels (Invitrogen, MA, USA) and transferred to a polyvinylidene difluoride (PVDF) membrane. Protein bands were detected using standard chemiluminescence techniques using GE Healthcare Amersham Imager 680.

ZNF71 antibody was purchased from GeneTex (Cat. No. GTX116553). The following antibodies were included in Western blots: ZEB1 (Sigma, St. Louis, MO, USA; HPA027524), Vimentin (V-9; Santa Cruz Biotechnology, TX, USA; sc-6260), E-cadherin (BD Biosciences, NJ, USA; cat #610181), pan-Cytokeratin (C11, Santa Cruz Biotechnology, Dallas, TX, USA; sc-8018), PCNA (eBioSciences, 14-6748-81), and Tubulin α (Sigma, St. Louis, MO, USA; T9026). Standard chemiluminescence was used to detect the protein bands.

### Statistical Methods

Various statistical methods can be utilized. Statistical analysis was performed using Rstudio version 1.4.1106. Differential gene expression between two groups was evaluated with Student's t-tests, and a two-sided p-value<0.05 was considered statistically significant. Survival analysis was performed using Kaplan-Meier analysis with the survival package in R. Log-rank tests were used to assess the difference in survival probability from different groups in Kaplan-Meier analyses. The genes in the networks were evaluated with Cox proportional hazard model and Kaplan-Meier analysis. Pearson's correlation test was used to find the relationship be-tween two variables. The multivariate Cox regression analysis was used to build the model of risk-scores. Differential gene mRNA and protein expression between two groups was evaluated with Student's t-tests, and a two-sided p-value<0.05 was considered statistically significant. Pearson's correlation test was used to find the relationship between two variables. One-tailed two proportion z-test was used to compare if two proportions are the same.

### Examples and Results

### Construction of Multi-omics CD27 Networks

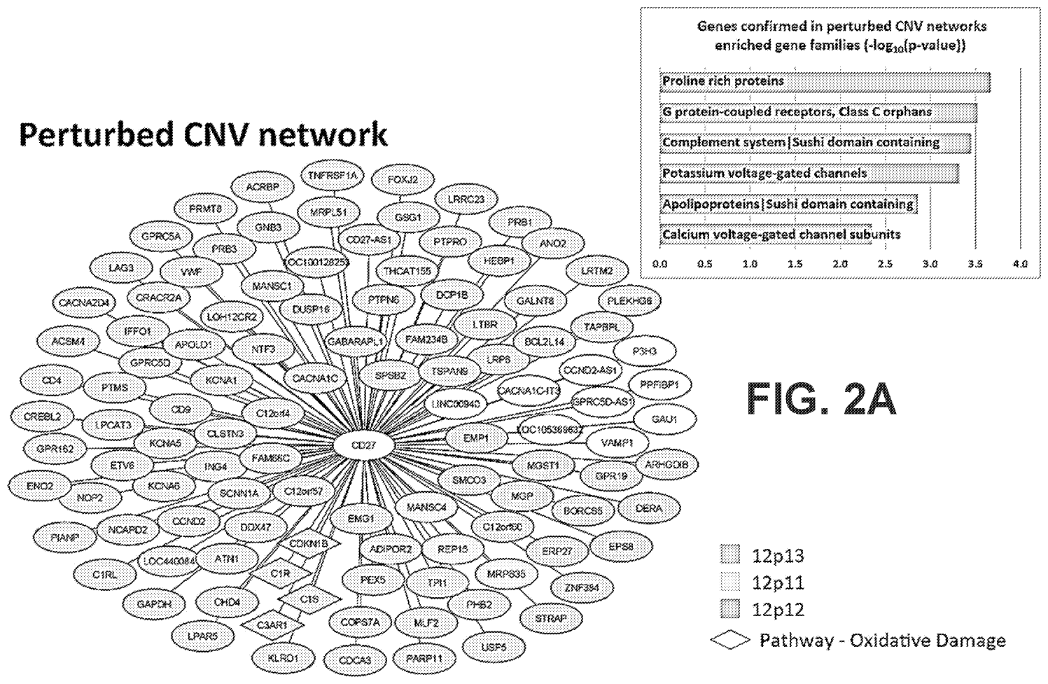

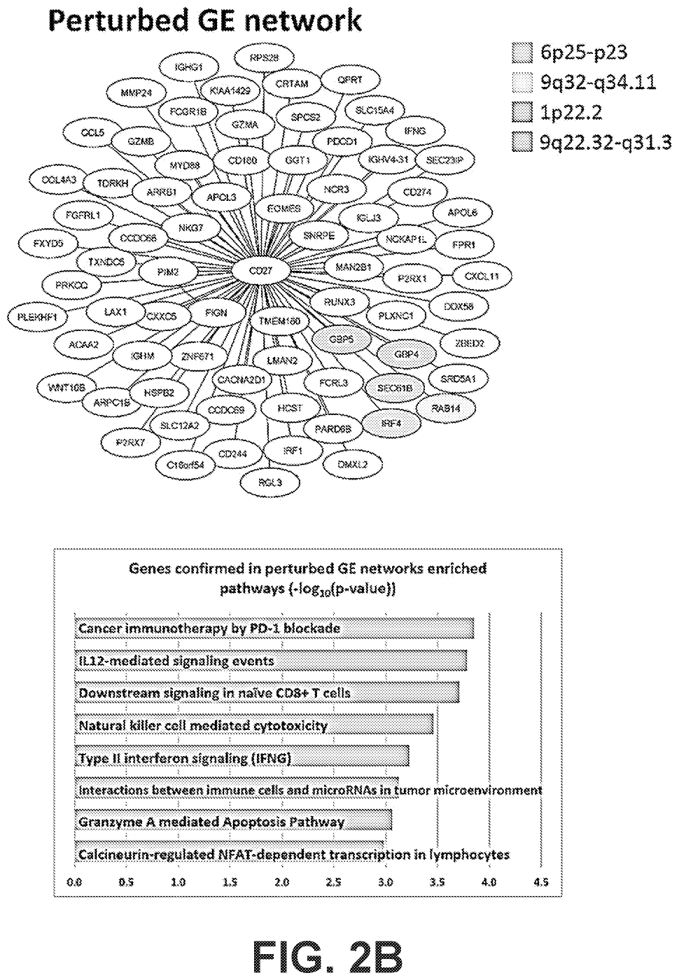

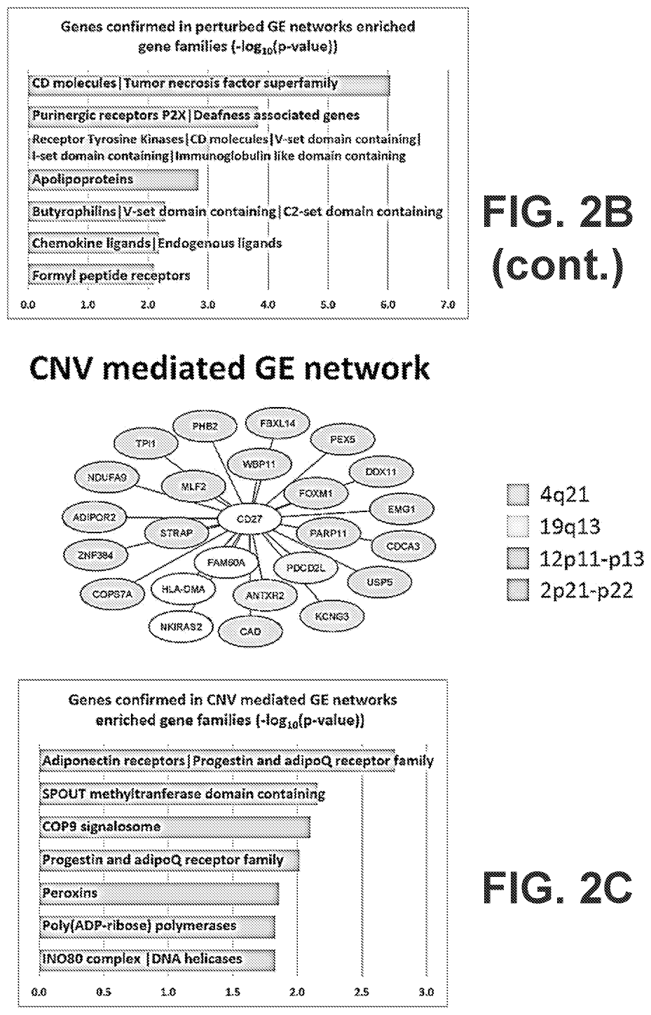

CD27 networks containing co-occurrences of DNA copy number aberrations, co-expression, and CNV mediated gene expression dysregulations were generated with two NSCLC patient cohorts (GSE31800 and GSE28582) using the Boolean implication network algorithm. The significant genetic associations (p<0.05, z-tests) found in both patient cohorts were kept for further analysis. There were 111 genes associated with CD27 from the CNV level, 75 genes from the gene expression level, and 24 genes from the CNV mediated gene expression (GE) level. FIGS. 2A-2C illustrate examples of CD27 multi-omics networks in NSCLC.

The enrichment of the CD27 networks in cytobands, gene families and pathways can be analyzed with ToppGene. The perturbed CD27 CNV network was overrepresented (p<0.05, FDR<0.05) in cytobands 12p11, 12p12 and 12p13. FIG. 2A illustrates an example of the perturbed CD27 CNV network. The plot shows the genes which had a significant (p<0.05, z-tests) concordant co-occurrence of DNA copy number aberration with CD27 in both patient cohorts GSE31800 and GSE28582. The bar chart shows the −log₁₀(p-values) of the significantly (p<0.05, FDR<0.05) enriched gene families in the ToppGene functional enrichment analysis. These genes were significantly (p<0.05, FDR<0.05) enriched in gene families including Proline rich proteins, G protein-coupled receptors, Class C orphans, Complement system, Potassium voltage-gated channels, Apolipoproteins, and Calcium voltage-gated channel subunits. The enriched (p<0.05, FDR<0.05) pathway was Oxidative Damage. Interestingly, LAG3, a promising ICI, had a significant (p<0.05, z-tests) co-amplification with CD27 in both patient cohorts GSE31800 and GSE28582. The computationally detected patterns showed when CD27 was amplified in the NSCLC tumors, LAG3 was also amplified; and when CD27 was not amplified in the NSCLC tumors, LAG3 was not amplified, either. Together with LAG3, numerous genes on cytoband 12p13 had a significant (p<0.05, z-tests) CNV co-occurrence with CD27.

FIG. 2B illustrates an example of the perturbed CD27 gene expression (GE) network. The plot shows the genes which had a significant (p<0.05, z-tests) concordant co-expression with CD27 in both patient cohorts GSE31800 and GSE28582. The bar charts show the −log₁₀(p-values) of the significantly (p<0.05, FDR<0.05) enriched pathways and gene families in the ToppGene analysis. The perturbed CD27 gene expression (GE) network of FIG. 2B was significantly (p<0.05, FDR<0.05) enriched in gene families including CD molecules/Tumor necrosis factor superfamily, Purinergic receptors P2X/Deafness associated genes, Receptor Tyrosine Kinases/CD molecules/Immunoglobulin like domain, Apolipoproteins, Butyrophilins, Chemokine ligands/Endogenous ligands, and Formyl peptide receptors. The enriched pathways (p<0.05, FDR<0.05) were Cancer immunotherapy by PD-1 blockade, IL 12-mediated signaling events, Downstream signaling in naïve CD8+ T cells, Natural killer cell mediated cytotoxicity, Type II interferon signaling (IFNG), Interactions between immune cells and microRNAs in tumor microenvironment, Granzyme A mediated Apoptosis Pathway, and Calcineurin-regulated NFAT-dependent transcription in lymphocytes. These genes were overrepresented (p<0.05, FDR<0.05) in cytobands 1p22.2, 6p25-p23, 9q32-q34.11, and 9q22.32-q31.3.

FIG. 2C illustrates an example of the CD27 CNV mediated gene expression (GE) network. The plot shows the genes which had a significant (p<0.05, z-tests) concordant CNV mediated gene expression regulation with CD27 in both patient cohorts GSE31800 and GSE28582. The bar chart shows the −log₁₀(p-values) of the significantly overrepresented gene families in the ToppGene analysis. The CD27 CNV mediated gene expression (GE) network of FIG. 2C was significantly (p<0.05, FDR<0.05) enriched in gene families including Adiponectin receptors, COP9 signalosome, Progestin and adipoQ receptor family, Peroxins, Poly(ADP-ribose) polymerases, and INO80 complex/DNA helicases. These genes were overrepresented (p<0.05, FDR<0.05) in cytobands 2p21-p22, 4q21, 12p11-p13, and 19q13. No significantly overrepresented pathways were found in the CD27 CNV mediated GE network.

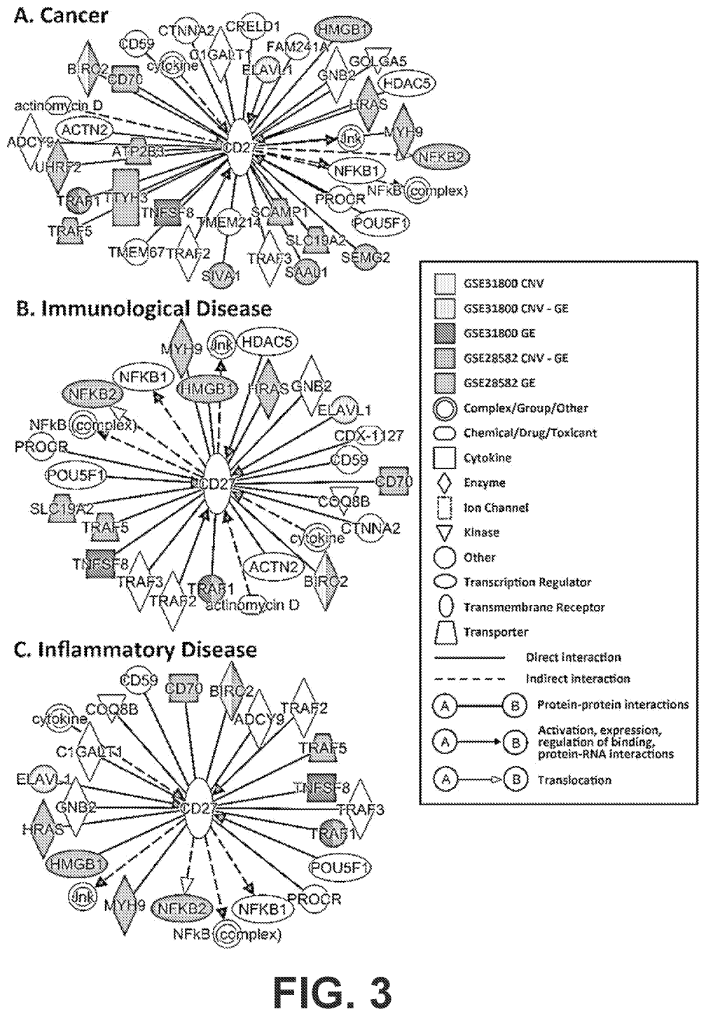

Multiple genes in the identified CD27 networks had experimentally confirmed interactions with CD27 as retrieved with IPA. FIG. 3 shows examples of the experimentally confirmed interactions with CD27 retrieved from IPA. Functional involvements in (A) cancer related interactions, (B) immunological disease related interactions, and (C) inflammatory disease related interactions are illustrated. In cancer, immunological disease, and inflammatory disease, CD27 activates NF-kB. Cytokines IL-2 and IL-15 down-regulate CD27. The binding and interaction of CD27 with CD70 make CD27-CD70 an attractive therapeutic target. In the Boolean implication networks generated in this study, a down-regulation of FCRL3 was associated with a down-regulation of CD27 and CD20 (MS4A1), respectively, in both patient cohorts GSE31800 and GSE28582 (results not shown). FCRL3 may be involved in human-specific mechanisms to control the generation of natural T regulatory (nTreg) cells, and its expression on nTreg cells correlates with cell hypoproliferation. The expression of FOXP3 alone is insufficient to induce FCRL3 expression. The co-expression associations among CD27, CD20, and FCRL3 observed in multiple NSCLC patient cohorts in this study warrant further investigation.

### Delineation of CD27, PD1, and PDL1 Multi-omics Networks

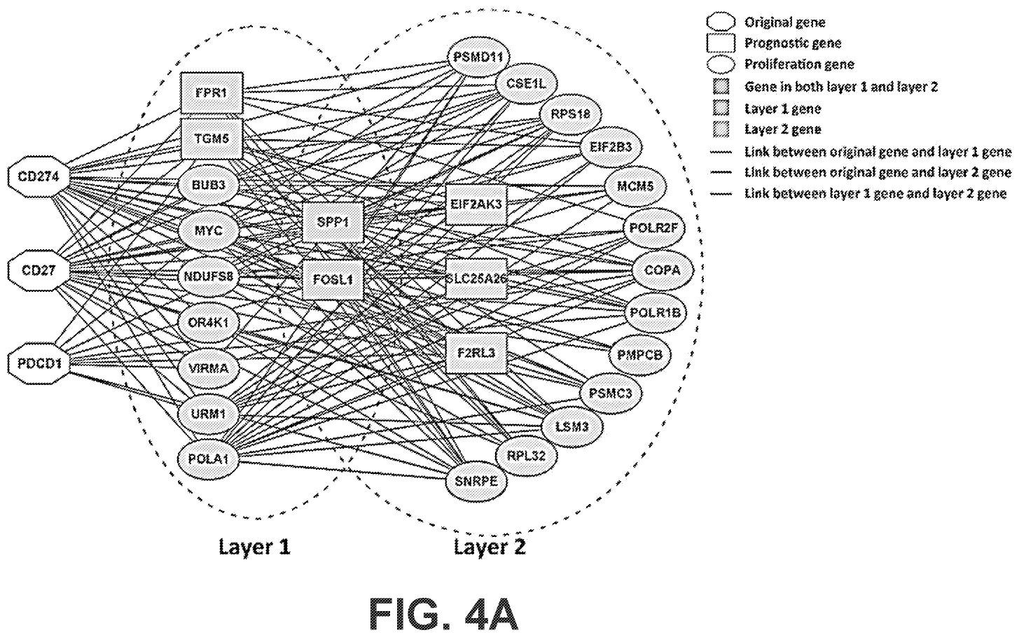

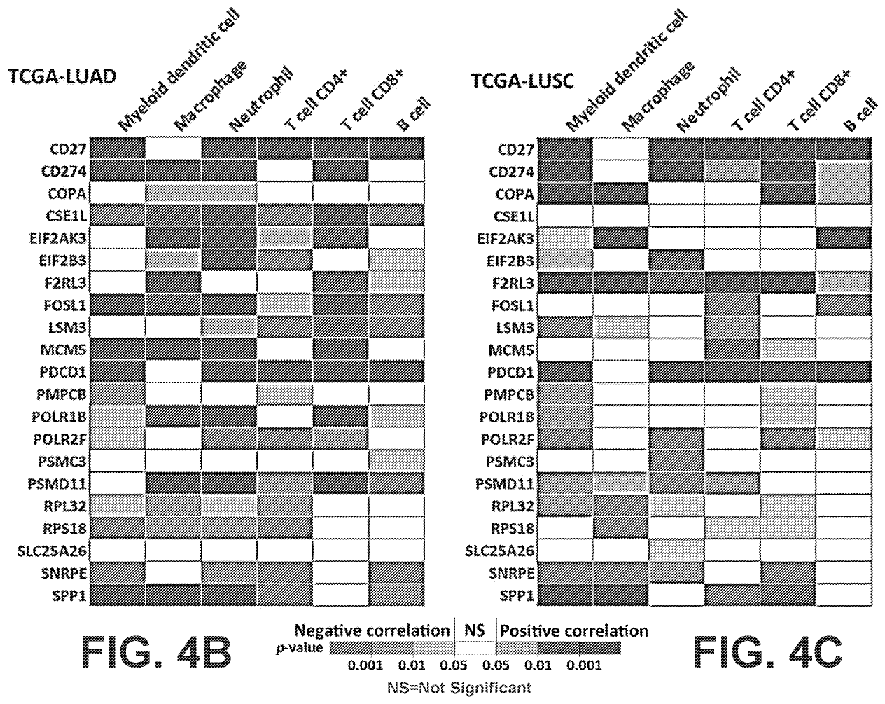

Multi-omics association networks of CD27, PD1 (PDCD1) and PDL1 (CD274) were also generated with the Boolean implication network algorithm. In the first layer, following genes were selected. First, genes that had a significant (p<0.05, z-tests) co-expression between CD27 and PD1, or between CD27 and PDL1, in both patient cohorts GSE31800 and GSE28582 were identified in the genome-scale analysis. Second, genes with a significant (p<0.05, z-tests) co-occurrence of CD27 and PDL1 DNA copy number aberrations in patient cohort GSE31800 were identified, since the CNV information of PDL1 was not available in GSE28582. Third, genes with a significant (p<0.05, z-tests) CNV mediated gene expression (GE) association between CD27 and PD1, or between CD27 and PDL1, in patient cohort GSE31800 were selected. There were 38 genes selected at the CNV level, 88 genes at the GE level, and 26 genes at the CNV mediated GE level. From this gene list, genes that were significantly associated with survival in GSE81089 (p<0.05, univariate Cox-modeling) and were validated with a concordant significant differential expression in short versus long survival (p<0.05, two sample t-tests) in GSE28582 were selected as prognostic genes in the layer 1, including SPP1, FPR1, TGM5, and FOSL1. FIG. 4A illustrates an example of a CD27, PD1, and PDL1 multi-omics association network.

FOSL1 connects the KRAS oncogene to components of the mitotic pathway. Mutant KRAS lung and pancreatic cancer patients with high FOSL1 expression had the worst survival outcome. FOSL1 inhibition is detrimental to both KRAS-driven lung and pancreatic cancer. MORAb-202, an anti-human FRα (FOSL1) antibody-drug conjugate achieved robust antitumor response in patient derived xenograft models and phase I study of FRα (FOSL1)-positive advanced solid tumors. SPP1 promotes proliferation, migration, invasion, and chemoresistance to cisplatin in NSCLC cells, and mediates macrophage polarization and facilitating immune evasion in lung adenocarcinoma through the upregulation of PDL1. In addition, genes with a significant dependency score in 50% of the tested NSCLC cell lines in CRISPR-Cas9 (n=78) or RNAi (n=92) were defined as layer 1 proliferation genes, including URM1, VIRMA, BUB3, POLA1, NDUFS8, OR4K1, and MYC. See, e.g., FIG. 4A.

Similarly, genes with a significant association (p<0.05, z-tests) with at least 2 out of the 14 genes (CD27, PD1, PDL1, and 11 layer 1 genes) in both patient cohorts GSE31800 and GSE28582 were selected as layer 2 candidate genes using the Boolean implication networks. A total of 30 genes at the CNV level, 1,538 genes at the GE level, and 24 genes at the CNV mediated GE level were selected. From this list of the layer 2 genes, 33 genes were significantly associated with survival in the patient cohort GSE81089 (p<0.05, univariate Cox-modeling) and were validated with a concordant significant differential expression in short versus long survival (p<0.05, two sample t-tests) in the patient cohort GSE28582. When fitting these 33 genes in a multivariate Cox model in a separate NSCLC patient cohort GSE81089 (n=197), 5 significant (p<0.05, multivariate Cox modeling) genes, including EIF2AK3, F2RL3, FOSL1, SLC25A26, and SPP1, were selected as layer 2 prognostic genes. From the list of the layer 2 candidate genes, 13 genes (COPA, CSE1L, EIF2B3, LSM3, MCM5, PMPCB, POLR1B, POLR2F, PSMC3, PSMD11, RPL32, RPS18, and SNRPE) had a significant dependency score in 100% of the tested NSCLC cell lines in both CRISPR-Cas9 (n=78) and RNAi assays (n=92), defined as layer 2 proliferation genes.

The final immune-omics network of FIG. 4A contained genes having a direct or indirect association with CD27, PD1, and PDL1, with each network edge indicating a co-occurrence of CNV, co-expression, or CNV mediated gene expression dysregulation in NSCLC tumors observed in multiple patient cohorts. This network contained both NSCLC prognostic genes and proliferation genes. Like CD27, PD1, and PDL1, the identified NSCLC prognostic genes and proliferation genes in the immune-omics network had a higher occurrence of CNVs than mutations in TCGA LUAD and LUSC tumors. The correlation of immune infiltration with the expression of 21 genes (CD27, PD1, PDL1 and the layer 2 genes) in TCGA-LUAD (n=515) and TCGA-LUSC (n=501) patient cohorts was assessed with TIMER. FIGS. 4B and 4C show the correlation of immune infiltration level with gene expression in the TCGA-LUAD patients (n=515) and the TCGA-LUSC patients (n=501) estimated with TIMER, respectively. The immune infiltration included myeloid dendritic cells, macrophages, neutrophils, CD4+ T cells, CD8+ T cells, and B cells.

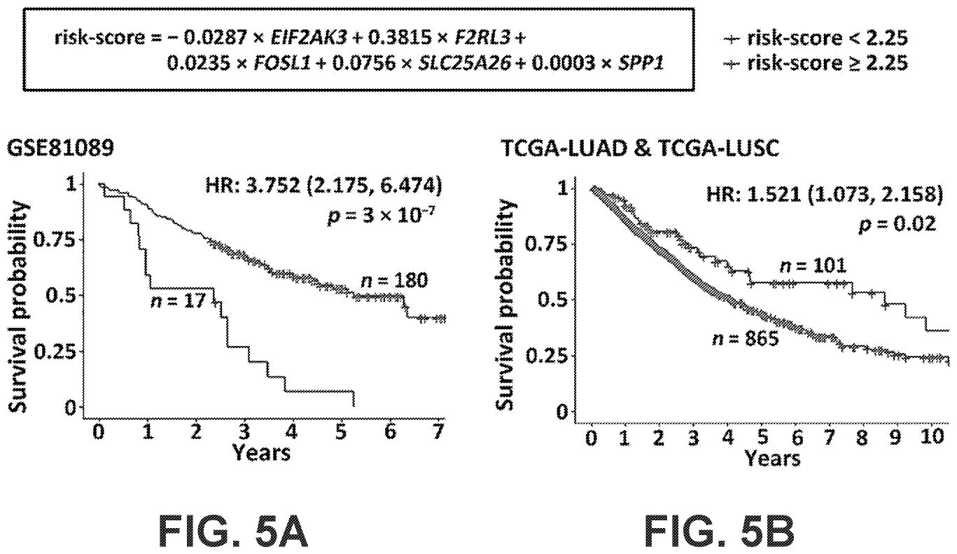

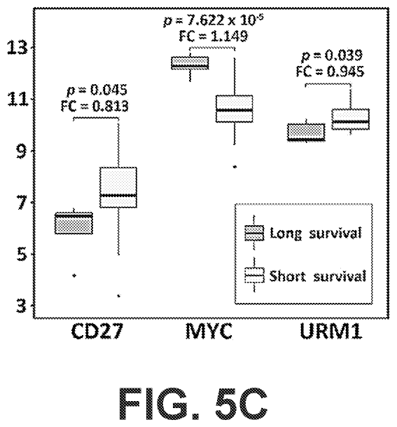

Using the 5 prognostic genes selected in the layer 2 of FIG. 4A, a multivariate Cox model was built in the patient cohort GSE81089 (n=197) to calculate the risk-score for each patient. The 5-gene prognostic model for NSCLC is illustrated in FIGS. 5A and 5B. FIG. 5A illustrates the Kaplan-Meier analysis of patients grouped by risk-scores with the cutoff value of 2.25 in GSE81089. The Kaplan-Meier analysis results showed that the patients who had a risk-score less than 2.25 survived significantly (p=3×10⁻⁷, HR: 3.752 [2.175, 6.474]) longer than the patients who had a risk-score equal or greater than 2.25 in the training cohort GSE81089. FIG. 5B illustrates the Kaplan-Meier analysis of patients stratified by risk-scores with the cutoff value of 2.25 in TCGA-LUSC and TCGA-LUAD. The plot shows the results of first 10 years after surgery. The mRNA expression data of TCGA-LUAD and TCGA-LUSC was used to validate this prognostic model. The Kaplan-Meier analysis results in the TCGA data (n=966) also showed a significantly better 10-year survival (p=0.02, HR: 1.521 [1.073, 2.158]) in the patient group with lower risk-scores, confirming the results from the training data.

Identification of Genes Associated with Radiotherapy and Chemotherapy

According to the current practice guidelines, NSCLC patients with stage II and above receive chemotherapy, with additional radiation for stage IIIA patients. To identify genes associated with radiotherapy response, stage III and IV patients in TCGA-LUAD and TCGA-LUSC who had received radiotherapy were included in the analysis. FIG. 5C illustrates genes associated with a radiotherapy response. These genes had a significant differential expression (p<0.05, two sample t-tests) in the long survival group versus the short survival group in TCGA-LUSC and TCGA-LUAD. Stage III or IV patients who had received radiotherapy were included in the analysis. (FC: fold change of gene expression in the long survival versus the short survival patient group).

Among the genes identified in the immune-omics network of FIG. 4A, CD27, MYC, and URM1 had a significant differential expression (p<0.05, two sample t-tests) in the short survival (<20 mo.) versus long survival (>58 mo.) patient group. It is recognized that many factors other than the response to radiotherapy may impact survival in this studied patient cohort. CD27 and URM1 expressed higher in the short survival patient group, and MYC expressed higher in the long survival patient group. The main function of URM1, a ubiquitin-like protein, is to modify tRNA, and elevated levels of tRNA modifying enzymes promote tumorigenesis and metastasis. The MYC oncogene causes many human cancers and is activated by FOXP3 in NSCLC.

Furthermore, drug responses of 10 commonly used chemotherapeutic regimens in treating NSCLC were included in this study, including carboplatin, cisplatin, paclitaxel (Taxol), pemetrexed, docetaxel, gemcitabine, vinorelbine, etoposide, gefitinib, and erlotinib. The studied NSCLC cell lines included adenocarcinoma, squamous cell carcinoma, large cell carcinoma, and adenosquamous carcinoma. Due to the synergism and successful results of the combination of cisplatin-etoposide in treating small cell lung cancer, long term daily administration of oral etoposide in combination with cisplatin was used to treat NSCLC. A systematic review showed that cisplatin-etoposide has comparable efficacy as carboplatin-paclitaxel when used with concurrent radiotherapy for patients with stage III unresectable NSCLC. Paclitaxel, a tubulin-binding agent, is commonly used to treat NSCLC in combination with a platinum-based compound. Gefitinib and erlotinib are widely used epidermal growth factor receptor (EGFR) tyrosine kinase inhibitors for treating advanced NSCLC with proven efficacy.

A recent meta-analysis showed that gefitinib and erlotinib have comparable effects on patient survival, overall response rate, and disease control rate, with no considerable variation associated with EGFR mutation status, ethnicity, line of treatment, and baseline brain metastasis status. Docetaxel offers clinical benefits as a second-line treatment of NSCLC in patients previously treated with platinum-based chemotherapy. The combination of pembrolizumab (anti-PD1 immunotherapy) plus docetaxel is well tolerated and substantially improved progression-free survival and overall response rate in patients with advanced NSCLC after platinum-based chemotherapy, including patients with EGFR variations. Here, genes associated with chemoresponse were selected based on their differential expression in sensitive versus resistant NSCLC cell lines to the studied drugs. In the identified CD27, PD1, and PDL1 network of FIG. 4A, multiple genes were associated with chemosensitivity or chemoresistance to cisplatin, docetaxel, erlotinib, etoposide, gefitinib, gemcitabine, paclitaxel, pemetrexed, and vinorelbine in the CCLE NSCLC cell lines (n=117, Table 1).

TABLE 1
Genes with a significant differential expression (p < 0.05; two sample t-tests) in sensitive
versus resistant CCLE NSCLC cell lines (n = 117) to specific drugs. Drug activity measurements
were natural log transformed. BOLD font indicates the gene has a higher expression in the
resistant cell lines than in the sensitive cell lines. REGULAR font indicates the gene
had a lower expression in the resistant cell lines than in the sensitive cell lines.

| | PRISM | | | |
|---|---|---|---|---|
| | In(IC₅₀) | PRISM In(EC₅₀) | GDSC1 In(IC₅₀) | GDSC2 In(IC₅₀) |
| Carboplatin | | | | |
| Cisplatin | | | | PSMD11 |
| Docetaxel | | RPS18 | EIF2AK3, | PMPCB, POLR1B |
| | | | PMPCB, PSMD11 | |
| Erlotinib | | LSM3, POLR2F, | CSE1L | FOSL1 |
| | | RPL32 | | |
| Etoposide | | PSMD11 | | |
| Gefitinib | | | COPA | |
| Gemcitabine | PSMC3 | PSMC3 | | SPP1, MCM5, |
| | | | | RPS18 |
| Paclitaxel | PSMD11 | PMPCB, RPS18 | | POLR1B |
| Pemetrexed | | | RPS18 | |
| Vinorelbine | | POLR2F | CSE1L, SNRPE | |

### Discovery of Repurposing Drug Candidates

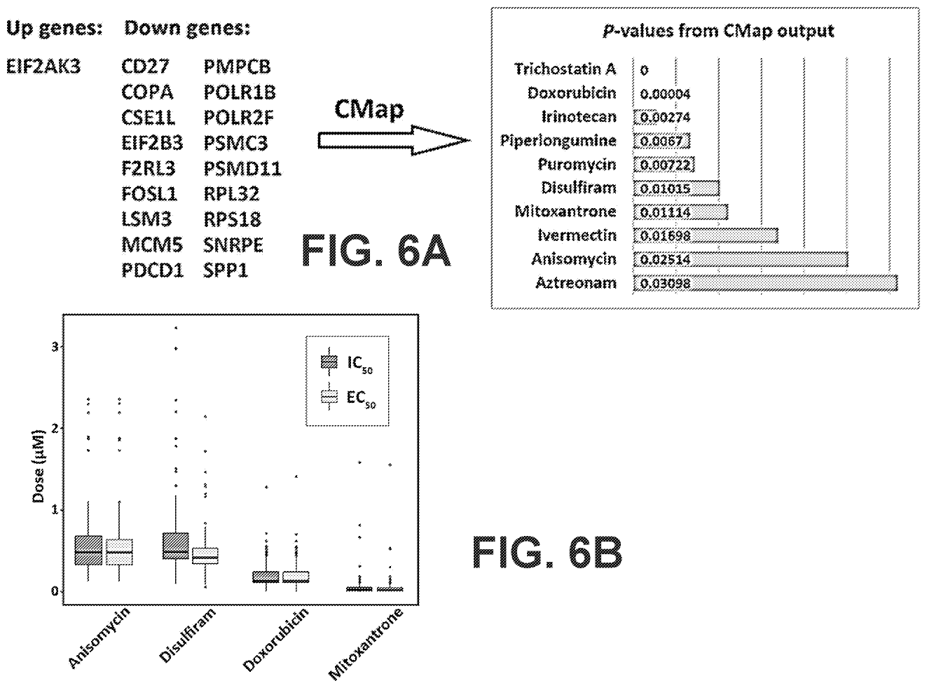

The identified CD27, PD1, and PDL1 network of FIG. 4A was used to discover repurposing drugs with potential implications in improving NSCLC treatment in combination with immunotherapy. One or more of the following mechanisms of action can be (and were) considered in the selection of small molecules: 1) to down-regulate CD27, PD1, and PDL1 expression; 2) to inhibit proliferation genes that had a significant impact in 100% of tested NSCLC cell lines in CRISPR-Cas9 and RNAi assays; 3) to maintain a high expression of survival protection genes that expressed higher in the long survival patient group; 4) to down-regulate the expression of survival hazard genes that expressed higher in the short survival patient group. The identified survival protection genes and hazard genes had consistent expression patterns in three NSCLC cohorts (GSE81089, GSE28582, and GSE81089) as well as TCGA. This gene expression signature was input into CMap to generate a list of significant compounds. FIGS. 6A and 6B illustrate an example of the discovery of repurposing drug candidates. FIG. 6A shows selection of small molecules based on the identified gene expression signature with CMap. PDL1 was not available in the CMap database and was not included in the input gene list in FIG. 6A.

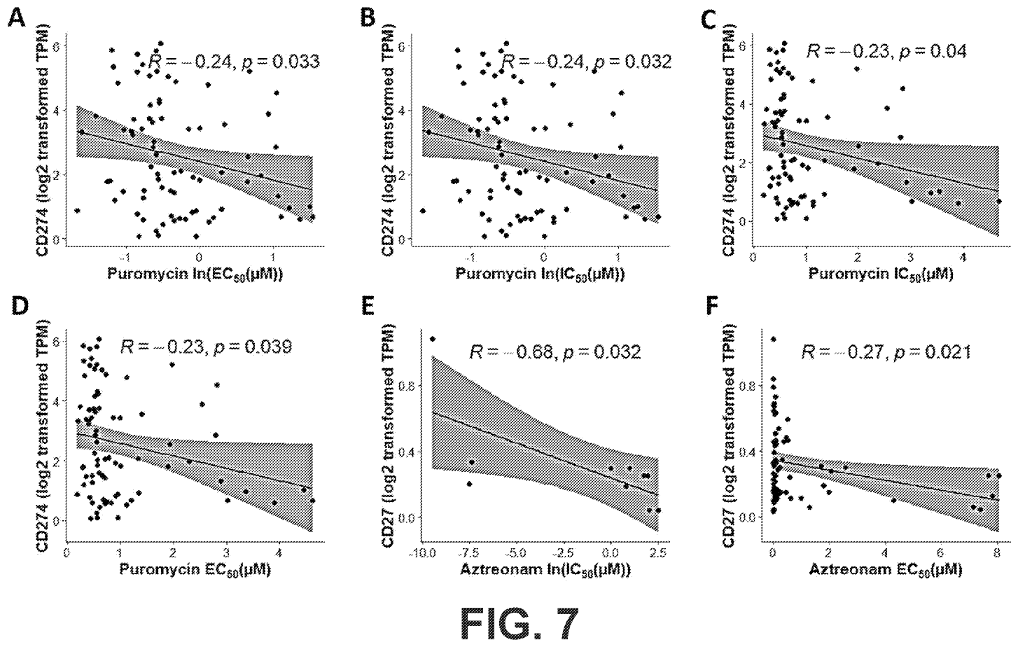

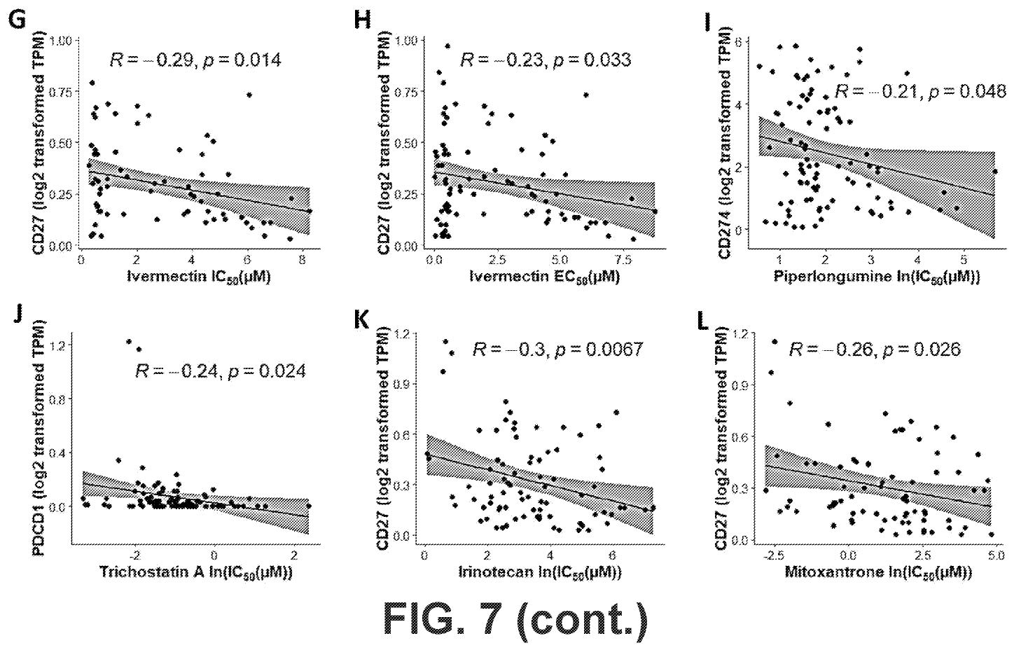

A total of 119 significant (p<0.05) compounds were identified with CMap. These compounds were further selected by using, e.g., the following criteria in the CCLE NSCLC panel (n=117): 1) choosing the small molecules with strong inhibitory effects of NSCLC cells, i.e., with low IC₅₀ and EC₅₀ values, and 2) filtering the compounds with a negative correlation of drug concentration and mRNA expression of CD27, PD1, or PDL1 in the NSCLC cell lines. In the original PRISM drug screen, 8 doses ranging from 0.0006 μM to 10 μM were tested on most of the compounds. Four small molecules (anisomycin, disulfiram, doxorubicin, and mitoxantrone) had a small average IC₅₀ or EC₅₀ value (<1 μM) in the PRISM drug screen and were selected for drug repurposing, since most NSCLC cell lines were relatively sensitive to these small molecules. FIG. 6B illustrates small molecules that had a low average concentration of drug response in the CCLE NSCLC cell lines (n=117). An outlier, disulfiram EC₅₀₌25.645, was excluded from the plot. Seven compounds (puromycin, aztreonam, ivermectin, piperlongumine, trichostatin A, irinotecan, and mitoxantrone) had a significant negative correlation (p<0.05, Pearson's correlation) of drug concentration [IC₅₀, EC₅₀, ln(IC₅₀), or ln(EC₅₀) value] with mRNA expression of CD27, PD1, or PDL1, respectively, in the datasets PRISM, GDSC1 or GDSC2. FIG. 7 illustrates examples of compounds with a significantly negative correlation of drug concentration and the mRNA expression of CD27, PD1, or PDL1 in the CCLE NSCLC cell lines (n=117). The drug activity measurements included IC₅₀, EC₅₀, ln(IC₅₀), and ln(EC₅₀).

Doxorubicin, mitoxantrone, irinotecan are chemotherapeutic drugs. Doxorubicin is used to treat breast cancer, bladder cancer, Kaposi's sarcoma, lymphoma, and acute lymphocytic leukemia. TIGAR knockdown may inhibit epithelial-to-mesenchymal (EMT) transition in doxorubicin-resistant human NSCLC. Mitoxantrone is mainly used to treat acute myeloid leukemia, with reported excellent in vitro antitumor activity against human lung adenocarcinoma. Irinotecan is used for the treatment of colon cancer and small cell lung cancer. A randomized phase III clinical trial showed that cisplatin plus irinotecan, carboplatin plus paclitaxel, cisplatin plus gemcitabine, and cisplatin plus vinorelbine, have similar efficacy and different toxicity profiles, and they can treat advanced NSCLC. The results from this study showed that doxorubicin, mitoxantrone, irinotecan may have inhibitory effects on CD27 and PD1 based on the CMap pharmacogenomic profiles, with potential implications in combined use with NSCLC immunotherapy.

Anisomycin, an antibiotic and a potential psychiatric drug, can activate stress-activated protein kinases, MAP kinase, and other signal transduction pathways. Disulfiram (Antabuse), a drug for alcohol use disorders, is a possible treatment for cancer, parasitic infections, and latent HIV infection. Puromycin is an antibiotic protein synthesis inhibitor critical in mRNA display. Aztreonam is an antibiotic mainly used to treat infections. Piperlongumine is a natural alkaloid from long peppers, which increases the level of reactive oxygen species and selectively kills cancer cells. Trichostatin A, an organic compound and an antifungal antibiotic, selectively inhibits class I and class II mammalian histone deacetylase (HDAC), but not class III HDAC. Ivermectin, a medication used to treat many types of parasite infestations, showed in vitro antiviral effects against several positive-sense single-strand RNA viruses, including SARS-CoV-2, with doses much higher than the maximum approved for safely use. These results provided evidence for drug repurposing to improve NSCLC treatment in combination with clinical therapies.

### Observations

Lung cancer has the highest cancer-related mortality in both men and women due to its complex etiology, advanced disease stage at the time of diagnosis, and a lack of biomarkers for the selection of chemotherapy, immunotherapy and radiotherapy in individual patients based on their tumor characteristics. Multiple ICIs have more CNV occurrences than mutations in NSCLC patient tumors. The mechanisms of CNV mediated transcriptional perturbations in the NSCLC disease course are not well understood. This study developed the novel Boolean implication network methodology to model integrated CNV and transcriptomic profiles in NSCLC tumors. The constructed immune-omics networks contain novel NSCLC proliferation genes selected from the CRISPR-Cas9 and RNAi screening data and prognostic genes validated in extensive patient cohorts. These identified genes are gnomically/transcriptomically associated with ICIs including CD27, PD1, and PDL1, and are linked to chemotherapy and radiotherapy response in NSCLC. The constructed immune-omics networks reveal information underlying molecular mechanisms of NSCLC, with potential implications in developing novel therapeutic strategies and discovering repositioning drug candidates to improve clinical therapies.

NSCLC tumor immune microenvironment is a complex molecular network functioning in multi-dimensional components of immune and stromal cells as well as blood vessels, exerting intricate interactions with epithelial tumor cells. To model this complicated molecular machinery, the Boolean implication network scheme was developed to integrate genome-scale CNV and gene expression profiles in NSCLC tumors. The presented Boolean implication network algorithm has the following advantages over other existing models. Firstly, it can analyze discrete CNV data and continuous gene expression data in a seamless, biologically relevant way that correlation networks cannot. Secondly, unlike the acyclic Bayesian networks, the presented Boolean implication networks can model cyclic molecular interactions, such as feedback loops. Further, the Boolean implication networks can model multivariate data based on prediction logic in combination with robust statistical tests, whereas other Boolean networks are primarily focused on binary variables. This is an improvement because categorized CNV and gene expression data should have at least three states to be biologically relevant, representing amplification/normal/deletion, or up-regulation/normal/down-regulation.

The Boolean implication networks can be utilized to model genome-scale co-expression networks and crosstalk with major NSCLC signaling pathways. The Boolean implication networks identified sets of biomarkers that generated an accurate prediction of lung cancer risk and metastases; meanwhile, Boolean implication networks revealed more biologically relevant molecular interactions than the Boolean networks, Bayesian networks, and Pearson's correlation networks. Based on the candidate genes identified with the Boolean implication networks from public microarray data, the NSCLC prognostic and chemo-predictive 7-gene qRT-PCR assay was developed using multiple patient cohorts collected from the US hospitals and was validated with a clinical trial JBR. 10. Included in this 7-gene assay, CD27 and ZNF71 had concordant mRNA and protein expression in NSCLC tumors. The study showed that ZNF71-KRAB, the KRAB isoform that is transcriptional repression, was associated with EMT in NSCLC tumors and cell lines. CD27, an emerging ICI, is being tested as adjuvant therapy in phase I/II clinical trials for multiple tumor types with promising results. These results demonstrate that embedding molecular networks and crosstalk with major NSCLC signaling hallmarks can identify biologically relevant biomarkers with prognostic and predictive implications and with potential as therapeutic targets.

Novel combinations of chemotherapy with ICIs complicate biomarker discovery and multiple complex interactions relevant to immune responses can be considered in this process. This can improve the Boolean implication networks to model multivariate CNV and gene expression profiles in NSCLC tumors and identified the immune-omics network of CD27, PD1, and PDL1. Among the identified 5 prognostic genes, anti-human FRα (FOSL1) antibody-drug conjugate (MORAb-202) achieved robust antitumor response in patient derived xenograft models and a phase I clinical trial of FRα (FOSL1)-positive advanced solid tumors. The identified NSCLC proliferation genes (COPA, CSE1L, EIF2B3, LSM3, MCM5, PMPCB, POLR1B, POLR2F, PSMC3, PSMD11, RPL32, RPS18, and SNRPE) had a significant impact on 100% of NSCLC cell lines in CRISPR-Cas9 and RNAi assays. TBP-1 (PSMC3) depletion stabilizes FRα (FOSL1) and further increases its levels in tumor cells expressing RAS-ERK pathway oncogenes. These results show that the proposed network approach reveals molecular interactions relevant to the underlying mechanisms.

The multi-omics network involving the 7-gene signature was dissected. All the network genes, including PD-L1 (CD274), had a significant correlation with immune infiltration in at least one immune cell types of TCGA NSCLC tumors, including myeloid dendritic cells, macrophages, neutrophils, CD4+ T cells, CD8+ T cells, and B cells. The mRNA and protein expressions of numerous network genes were associated with drug sensitivity or resistance to 10 commonly used therapeutic regimens for treating NSCLC. The multi-omics network genes had a higher percentage (82.9%) of correlated mRNA and protein expression in CCLE NSCLC cell lines compared with the rest of the genome (66.3%). These results further substantiate the potential clinical implications of the multi-omics network of the 7-gene signature.

The Golgi apparatus, M-COPA (2-methylcoprophilinamide), might be a promising therapeutic target to conquer the detrimental cycle of TKI resistance in EGFR-mutated NSCLC cells via downregulating cell surface RTK expression. CSE1L promotes the nuclear accumulation of transcriptional coactivator TAZ and enhances invasiveness and malignancy in human lung cancer and glioblastoma cells. Depletion of ore spliceosome encoding genes SNRPE or SNRPD1 remarkably reduces cell viability in breast, lung, and melanoma cancer cell lines. Knockdown of SNRPE leads to dramatically decreased mTOR mRNA and protein levels, accompanied with deregulated mTOR pathway. Together, these results demonstrate that the multi-omics network approach unveils promising therapeutic strategies and novel linkages to ICIs including CD27, PD1, and PDL1. The association of the identified genes with chemoresponse and radiotherapy response revealed in this study can aid clinical studies toward precision oncology.

Drug repositioning can improve patient care, due to a lower risk of failure, expediated drug development, and less investment. The presented network approach, in combination with comprehensive CRISPR-Cas9/RNAi screening and extensive patient gene expression/survival profiles, discovered promising drug repurposing candidates for treating NSCLC. Three chemotherapeutic agents were recommended for repurposing in this study, doxorubicin, mitoxantrone, and irinotecan. This study provided the first evidence that these agents had inhibitory effects on CD27 and PD1 based on the CMap pharmacological database and the CCLE NSCLC panel. Cisplatin plus irinotecan was proven as effective in treating advanced NSCLC in a randomized phase III clinical trial, substantiating the validity of the presented approach. The identified antibiotic compounds (anisomycin, puromycin, aztreonam, trichostatin A, and ivermectin), natural compound Piperlongumine, and disulfiram (Antabuse) warrant further study as repositioning candidates for treatment of NSCLC, based on their relevant pharmacological profiles in the CMap database and their inhibitory effects on the CCLE NSCLC cell lines.

The present disclosure identifies a CNV mediated transcriptional regulatory network of CD27, PD1, and PDL1 with substantiation in multiple NSCLC cohorts using Boolean implication networks. This immune-omics network contained prognostic genes validated in extensive patient cohorts and proliferation genes having a significant impact on 100% of NSCLC cell lines in CRISPR-Cas9 and RNAi assays. A 5-gene prognostic model was derived and validated for patient stratification in multiple cohorts including TCGA data. Numerous identified genes were associated with chemoresponse and radiotherapy response in NSCLC cell lines and patient tumors. The constructed immune-omics network revealed promising therapeutic strategies and novel linkages to ICIs including CD27, PD1, and PDL1. This study provided evidence that doxorubicin, mitoxantrone, and irinotecan had inhibitory effects on CD27 and PD1. Drug repurposing candidates were discovered for improving lung cancer treatment and pharmaceutical R&D.

Integrated CNV and transcriptional profiles in NSCLC tumors (n=371) were analyzed using the Boolean implication networks for the identification of a multi-omics CD27, PD1, and PDL1 network, containing novel prognostic genes and proliferation genes. A 5-gene (EIF2AK3, F2RL3, FOSL1, SLC25A26, and SPP1) prognostic model was developed and validated for patient stratification (p<0.02, Kaplan-Meier analyses) in NSCLC tumors (n=1,163). A total of 13 genes (COPA, CSE1L, EIF2B3, LSM3, MCM5, PMPCB, POLR1B, POLR2F, PSMC3, PSMD11, RPL32, RPS18, and SNRPE) had a significant impact on proliferation in 100% of the NSCLC cell lines in both CRISPR-Cas9 (n=78) and RNA interference (RNAi) assays (n=92). Multiple identified genes were associated with chemoresponse and radiotherapy response in NSCLC cell lines (n=117) and patient tumors (n=966). Repurposing drugs were discovered based on this immune-omics network to improve NSCLC treatment.

In some embodiments, the nucleic acid sequence for EIF2AK3 can be found in NCBI Accession No. NG_016424 (5113 . . . 75836). In some embodiments, the nucleic acid sequence for F2RL3 can be found in NCBI Accession No. NG_032894 (4985 . . . 8592). In some embodiments, the nucleic acid sequence for FOSL1 can be found in NCBI Accession No. NC_000011 (complement of 65892049 . . . 65900545). In some embodiments, the nucleic acid sequence for SLC25A26is can be found in NCBI Accession No. NG_054637 (5001 . . . 250318). In some embodiments, the nucleic acid sequence for SPP1 can be found in NCBI Accession No. NG_030362 (5001 . . . 12762). In some embodiments, the nucleic acid sequence for CD27 can be found in NCBI Accession No. NG_031995 (5001 . . . 11834). In some embodiments, the nucleic acid sequence for MYC can be found in NCBI Accession No. NG_007161 (5798 . . . 12518). In some embodiments, the nucleic acid sequence for URM1 can be found in NCBI Accession No. NC_000009 (128371319 . . . 128392016). In some embodiments, the nucleic acid sequence for PSMD11 can be found in NCBI Accession No. NC_000017 (32444510 . . . 32483319). In some embodiments, the nucleic acid sequence for RPS18 can be found in NCBI Accession No. NC_000006 (33272075 . . . 33276511). In some embodiments, the nucleic acid sequence for PMPCB can be found in NCBI Accession No. NC_000007 (103297435 . . . 103329902). In some embodiments, the nucleic acid sequence for POLR1B can be found in NCBI Accession No. NC_000002 (112542036 . . . 112579818). In some embodiments, the nucleic acid sequence for LSM3 can be found in NCBI Accession No. NC_000003 (14178817 . . . 14201122). In some embodiments, the nucleic acid sequence for POLR2F can be found in NCBI Accession No. NC_000022 (37953663 . . . 38041915). In some embodiments, the nucleic acid sequence for RPL32 can be found in NCBI Accession No. NC_000003 (complement of 12834485 . . . 12841582). In some embodiments, the nucleic acid sequence for CSE1L can be found in NCBI Accession No. NC_000020 (49046312 . . . 49096949). In some embodiments, the nucleic acid sequence for COPA can be found in NCBI Accession No. NG_050927(5315 . . . 59971). In some embodiments, the nucleic acid sequence for PSMC3 can be found in NCBI Accession No. NC_000011 (complement of 47418775 . . . 47426473). In some embodiments, the nucleic acid sequence for MCM5 can be found in NCBI Accession No. NC_000022 (35400133 . . . 35454942). In some embodiments, the nucleic acid sequence for SNRPE can be found in NCBI Accession No. NG_050954 (5001 . . . 14568).

Lung cancer is the second most common cancer and the leading cause of cancer deaths worldwide, accounting for nearly 25% of all cancer deaths. An estimate from the American Cancer Society shows that in 2021, 235,760 adults (119,100 men and 116,660 women) in the US were diagnosed with lung cancer. In the US, 60%-65% of newly diagnosed lung cancer cases will be never-smokers or former smokers. About 84% of all known lung cancers cases are non-small-cell lung cancer (NSCLC). Major histological subtypes of NSCLC include lung adenocarcinoma (LUAD), squamous cell carcinoma (LUSC), and large cell carcinoma, while LUAD and LUSC accounting for nearly 50% and 30% of NSCLC, respectively. The overall 5-year survival rate after initial diagnosis for NSCLC is less than 15% due to the limited therapeutic response, recurrence, extensive invasion, and metastasis.

NSCLC patients with stage II and above receive chemotherapy, patients with stage III and IV receive additional radiotherapy. Although adjuvant chemotherapy for stage II and stage III disease improves overall survival rate by 10-15%, the prognosis of early-stage NSCLC is still poor, with a 5-year mortality rate of 40% in stage 1, 66% in stage II, and 75% in stage IIIA, as a result of recurrence. These data indicate that many patients may not benefit from prescribed chemotherapy. The FDA recently approved atezolizumab for stage II and IIIA NSCLC after platinum-based chemotherapy in patients with PD-L1>1%.

Currently, physicians do not have a precise tool to identify patients with early-stage NSCLC who are likely to develop recurrence or to predict the clinical benefits of chemotherapy, with or without subsequent immunotherapy depending on their PD-L1 expression levels.

A qRT-PCR based 7-gene microfluidic assay has been developed, including ABCC4, CCL19, SLC39A8, CD27, FUT7, DAG1, and ZNF71, for NSCLC prognosis and prediction of chemotherapeutic benefits. The ability of this gene assay to inform selection of specific chemotherapy for individual patients may improve cancer care. The protein expression of CD27 quantified with ELISA had a strong correlation with its mRNA expression in NSCLC tumors. CD27 is a new generation of immune checkpoint inhibitor (ICI). and is being tested as adjuvant therapy in phase I/II clinical trials for multiple tumor types with promising results. Increasing evidence shows that CD27 agonist antibodies, either alone or in combination with PD1-blockade, can improve the therapeutic efficacy of cancer vaccines and immunotherapy in general. zinc finger protein 71 (ZNF71) protein expression quantified with AQUA was identified as a prognostic marker of NSCLC. The Krüppel associated box (KRAB) is a transcriptional repression domain and is commonly present in human zinc finger protein-based transcription factors, i.e., KRAB zinc finger proteins (KRAB-ZFPs). Transcriptional repression mediated by KRAB-ZFPs is linked to cell proliferation, differentiation, apoptosis, and cancer. Emerging evidence suggests epithelial to mesenchymal transition (EMT) is involved in tumor progression, metastasis, and resistance to cancer therapy. ZNF71 KRAB isoform was found to be associated with EMT in NSCLC tumors and cell lines.

Revelation of genomic and transcriptomic landscape has shed lights to NSCLC subtypes and treatment. An examination of The Cancer Genome Atlas (TCGA) multi-omics data indicated that these seven prognostic genes have more DNA copy number variations (CNV) than mutations in NSCLC tumors. The present disclosure investigates 1) the feasibility of applying the 7-gene signature for NSCLC prognosis using next-generation sequencing (NGS) data of TCGA; and 2) the potential functional mechanisms involving the molecular networks of these seven genes. A novel Boolean implication network scheme was developed to model whole-genome CNV and gene expression profiles in NSCLC tumors (n=371) to dissect the molecular networks involving these seven prognostic genes. Based on the identified multi-omics network of the 7-gene signature, proliferation genes were identified from CRISPR-Cas9 (n=78) and RNAi (n=92) screening data. The identified network genes were further examined for their association with response to radiotherapy in TCGA patient tumors (n=966) and 10 commonly used NSCLC therapeutic regimens in the Cancer Cell Line Encyclopedia (CCLE) NSCLC panel (n=117). The immune infiltration associated with the identified genes was investigated in myeloid dendritic cells, macrophages, neutrophils, CD4+ T cells, CD8+ T cells, and B cells. The association between mRNA expression and protein expression of these network genes in NSCLC CCLE panel and TCGA tumors was analyzed.

EMT is a highly dynamic process, in which epithelial cells can convert to a mesenchymal phenotype. EMT is also reversible by the mesenchymal-to-epithelial transition (MET). The role of EMT in cancer patient outcomes is not well understood and remains controversial. A link between KRAB-ZFPs and EMT was reported. Tripartite motif containing 28 (TRIM28) protein, a universal co-factor for KRAB-ZFP transcription factors, contributes to EMT and might be relevant for tumor metastasis in lung cancer. ZNF71 KRAB was associated with patient outcome and with EMT, i.e., expressed higher in top 50% ZEB1 expressing cells. A mechanistic link between ZNF71 KRAB and EMT is not known. To gain a better understanding of the molecular mechanisms of ZNF71 and its isoforms, this study investigated the correlation between ZNF71 protein expression and EMT markers in NSCLC cell lines. Furthermore, molecular networks formed with proliferation genes co-expressed with ZNF71, ZNF71 KRAB, and ZNF71 KRAB-less isoforms in NSCLC tumors were constructed with the Boolean implication network algorithm, respectively. The gene expression of these proliferation networks was further analyzed with L1000 Connectivity Map (CMap) to identify matched functional pathways in shRNA knock-down, CRISPR-Cas9 knock-out, or overexpression assays in NSCLC cell lines. Further, the 14-gene EMT classifier constructed for characterizing NSCLC tumor EMT states. was combined with the identified multi-omics network of the 7-gene signature to generate mechanistic hypotheses and discover repositioning drugs to reverse EMT, to enhance drug sensitivity, and to inhibit ICIs and NSCLC proliferation. Such a revelation will provide insight for designing therapeutic strategies for NSCLC intervention in future research.

This study identified a multi-omics network of a patent pending 7-gene prognostic and predictive signature for NSCLC. The network genes contained proliferation genes identified from CRISPR-Cas9 and RNA interference (RNAi) screening data in the Cancer Cell Line Encyclopedia (CCLE) NSCLC panel. The network genes were associated with immune infiltration, radiotherapy outcomes in patient tumors, and drug sensitivity or resistance to 10 commonly used therapeutic regimens for treating NSCLC. This multi-omics network led to the discovery of re positioning drugs for improving lung cancer treatment.

Novel combinations of chemotherapy with immunotherapy complicate biomarker discovery and multiple complex interactions relevant to immune responses should be considered in this process. NSCLC tumor immune microenvironment is a complex molecular network functioning in multi-dimensional components of immune and stromal cells as well as blood vessels, exerting intricate interactions with epithelial tumor cells. To model this complicated molecular machinery, the Boolean implication network scheme was developed to integrate genome-scale CNV and gene expression profiles in NSCLC tumors. The multi-omics network involving the 7-gene signature was dissected. All the network genes, including PD-LI (CD274), had a significant correlation with immune infiltration in at least one immune cell types of TCGA NSCLC tumors, including myeloid dendritic cells, macrophages, neutrophils, CD4+ T cells, CD8+ T cells, and B cells. The mRNA and protein expressions of numerous network genes were associated with drug sensitivity or resistance to 10 commonly used therapeutic regimens for treating NSCLC. The multi-omics network genes had a higher percentage (82.9%) of correlated mRNA and protein expression in CCLE NSCLC cell lines compared with the rest of the genome (66.3%). These results further substantiate the potential clinical implications of the multi-omics network of the 7-gene signature.

The presented Boolean implication network algorithm is conceptually innovative and has several advantages over other methodologies. 1) It can analyze discrete CNV data and continuous gene expression data in a seamless and biologically relevant manner that correlation networks cannot; 2) Unlike the acyclic Bayesian networks, the Boolean implication networks can model molecular circuits, such as feedback loops, which are relevant in biological switches and signaling motifs that regulate large-scale cellular processes; and 3) the Boolean implication networks can model multivariate data based on prediction logic in combination with robust statistical tests, whereas other Boolean networks are primarily focused on binary variables. This is an improvement because categorized CNV and gene expression data should have at least three states to be biologically relevant, representing amplification/normal/deletion, or upregulation/normal/downregulation.

Pathway and network approaches will lead to the discovery of novel therapeutic targets as well as repositioning drug candidates. Drug repositioning can improve patient care, due to a lower risk of failure, expediated drug development, and less investment. To design therapeutic strategies to reverse epithelial to mesenchymal transition (EMT), enhance drug sensitivity, and inhibit NSCLC proliferation, significant functional pathways and repositioning drugs were identified with LIOOO CMap. The 14-gene EMT classifier to characterize NSCLC tumors, the multi-omics network of the 7-gene signature, and CRISPR Cas9/RNAi screening data were used to generate up-and down-regulated gene lists as input to CMap. ShRNA Knock down of the following pathways were linked to these mechanisms, including KD_APOLIPO PROTEINS, KD_CYCLINS, KD_LYSINE_ACETYLTRANSFERASES, KD_NO N_HOMOLOGOUS_EN D_JOINING, KD_V_TYPE_ATPASES, and

OE_NFKB_ACTIVATION. Nuclear factor kappa B (NF-kB) is a master regulator in NSCLC pathogenesis and progression. Activated NF-kB signaling may influence the progression of a lung tumor either positively via triggering immune surveillance or negatively via promoting inflammation and tumorigenesis. Activation of NF-kB stimulates transcription of cyclin D1, a key regulator of GI checkpoint control. NNK, a tobacco-specific ni trosamine, can stimulate proliferation through activation of the NF-kB and subsequent upregulation of cyclin 01 in a normal human bronchial cell. Inhibition of NF-kB results in loss of induction of EMT master-switch transcription factors in NSCLC. NF-kB activation has been associated with resistance to chemotherapeutics and tyrosine kinas e inhibitors in EGFR mutant lung cancer. Protein expression of V-ATPase is associated with drug resistance and higher tumor grade in NSCLC. Dasatinib and lestaurtinib used for leukemia treatment were identified as repurposing drugs for NSCLC. Homosalate, FK 888, and penfluridol were discovered as potential repositioning drugs that can kill NSCLC cells, maintaining the expression of drug sensitivity genes and epithelial markers, and inhibit mesenchymal markers, proliferation genes, and immune checkpoint inhibitors for NSCLC treatment.

Currently, there are no clinically available biomarkers to predict the risk for tumor recurrence and the clinical benefits of chemotherapy in early-stage NSCLC patients of all histological subtypes. There are insufficient biomarkers to select specific therapeutic regimens that individual advanced NSCLC patients might respond to. Studies showed that immune checkpoint inhibitors in combination with chemotherapy could improve NSCLC patient survival regardless of PD-LI expression. PD-LI and tumor mutational burdens are not proven indicative biomarkers. To date, predictive biomarkers of immunotherapy are not well established except PDI or PD-LI, and it is unlikely that a single marker is sufficient.

### Results

### A Multi-Omics Network of the 7-Gene NSCLC Prognostic and Predictive Signature

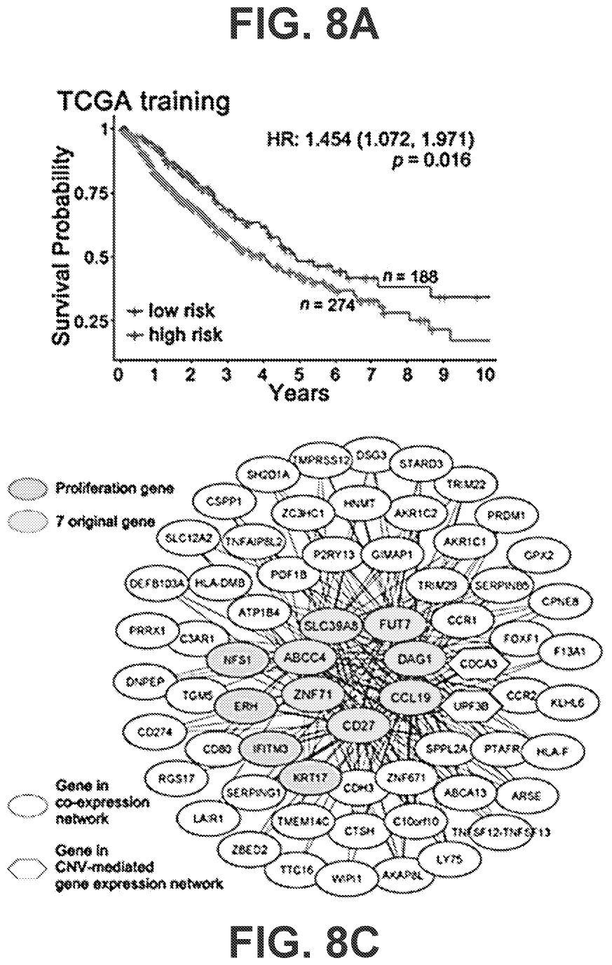

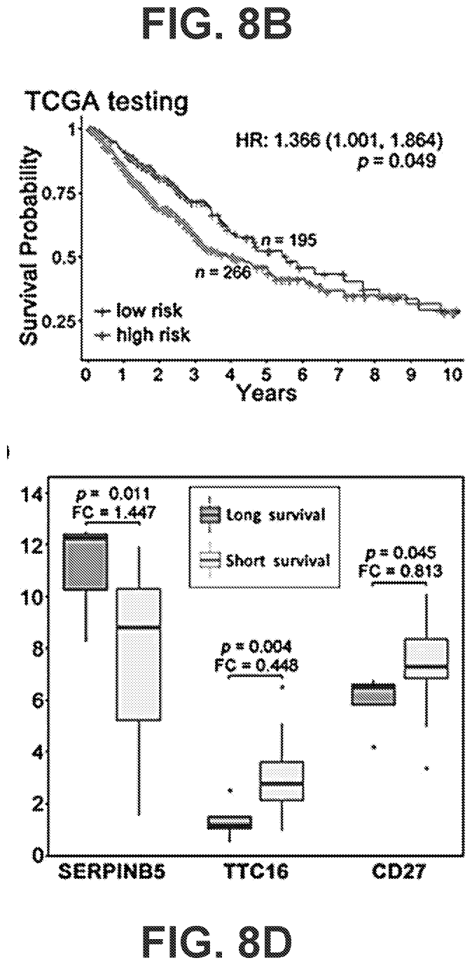

FIGS. 8A-D illustrate examples of a 7-gene signature and associated multi-omics network. FIG. 8A shows a Kaplan-Meier analysis of 7-gene signature in training set randomly partitioned from the combined TCGA-LUSC and TCGA-LUAD data. FIG. 8B shows a Kaplan-Meier analysis of 7-gene signature in testing set randomly partitioned from the combined TCGA-LUSC and TCGA-LUAD data. Patients were grouped by risk-scores with the cutoff value of −0.74. The plots show the survival outcome of the first 10 years after surgery.

A 7-gene NSCLC prognostic and chemopredictive qRT-PCR microfluidic assay can include ABCC4, CCL19, SLC39A8, CD27, FUT7, DAG1, and ZNF71. Here, whether it is feasible to apply this 7-gene signature in NGS data for NSCLC prognosis was investigated. TCGA-LUAD (n=515) and TCGA-LUSC (n=501) datasets were combined as TCGA NSCLC data (n=923), keeping patient samples with sufficient survival information. The combined TCGA NSCLC dataset was randomly partitioned into a training (n=462) and a testing set (n=461), for building and validating the prognostic model, respectively. A multivariate Cox-model was built using the 7 prognostic genes in the training set to calculate coefficients for the gene covariates and the risk-score for each patient. This model was applied to the testing set using the same gene coefficients and cutoff for patient stratification. The Kaplan-Meier analysis results in FIGS. 8A-B showed that the patients who had a risk-score less than −0.74 survived significantly longer than the patients who had a risk-score equal or greater than −0.74 in both training and testing sets (p=0.016, HR: 1.454 [1.072, 1.971] in training set, p=0.049, HR: 1.366 [1.001, 1.864] in the testing set; FIGS. 8A-B) in Kaplan-Meier analysis.

FIG. 8C shows the multi-omics network of the 7-gene signature in NSCLC tumors. All proliferation genes in the networks were identified from CRISPR-Cas9/RNAi screening data in CCLE NSCLC cell lines. FIG. 8D shows genes associated with radiotherapy response. These genes had a significant differential expression (p<0.05, two-sample t-tests) in the long survival (>58 months; n=144) group versus the short survival (<20 months; n=186) group in TCGA-LUSC and TCGA-LUAD. Stage III or IV patients who had received radiotherapy were included in the analysis.

A multi-omics association network of ABCC4, CCL19, SLC39A8, CD27, FUT7, DAG1, and ZNF71 were generated with the Boolean implication network algorithm. In this example, the following genes were selected to build the network. First, genes that had a significant (p<0.05, z-tests) co-expression with at least 2 of the 7 original genes in both patient cohorts GSE31800 and GSE28582 were identified in the genome-scale analysis. Second, genes with a significant (p<0.05, z-tests) CNV-mediated gene expression (GE) association with at least 2 of the 7 original genes in both patient cohorts GSE31800 and GSE28582 were identified. Third, genes with a significant (p<0.05, z-tests) co-occurrence of DNA copy number aberrations with at least 2 of the 7 original genes were searched for in both patient cohorts GSE31800 and GSE28582, but no genes were found. In addition, genes with a significant dependency score in 50% of the tested NSCLC cell lines in CRISPR-Cas9 (n=78) or RNAi (n=92) were defined as proliferation genes, including ERH, IFITM3, NFS1, and KRT17.

As shown in FIG. 8C, the final multi-omics network contained genes having an association with at least 2 of the 7 prognostic genes, with each edge indicating a co-expression or CNV-mediated gene expression dysregulation in NSCLC tumors observed in multiple patient cohorts. PD-L1 (CD274) was co-expressed with multiple signature genes. Down-regulation of CD274 was associated with up-regulation of DAG1 and down-regulation of DAG1 was associated with up-regulation of CD274. Up-regulation of CD274 was associated with up-regulation of CCL19 and down-regulation of CD27. X-ray irradiation induced miR-574-3p expression in NSCLC A549 cells, which in turn suppressed Enhancer Of Rudimentary Homolog (ERH) protein production and delayed cell growth. Interferon induced transmembrane protein 3 (IFITM3) protein expression was mainly detected in the cytoplasm of advanced lung adenocarcinoma tumor tissues and was correlated with higher tumor grade. Single-cell sequencing profiles of IFITM3 and ISG15 characterize an interferon signaling cluster of macrophages in metastasis-bearing lungs. NFS1 cysteine desulfurase is under positive selection in lung adenocarcinomas and protects cancer cells from ferroptosis, a non-apoptotic form of cell death, in response to oxidative damage. Keratin 17 (KRT17), reported to promote lung adenocarcinoma via enhancing cell proliferation and invasion, showed a paradoxical anti-metastatic role of KRT17 both in vitro and in vivo in cancer xenografts in a single-cell lineage study. This supports the relevance of the identified multi-omics network of the 7-gene signature in NSCLC.

According to the current practice guidelines, NSCLC patients with stage III and IV receive radiation therapy. To identify genes associated with radiotherapy response, stage III and IV patients in TCGA-LUAD and TCGA-LUSC who had received radiotherapy were included in the analysis.

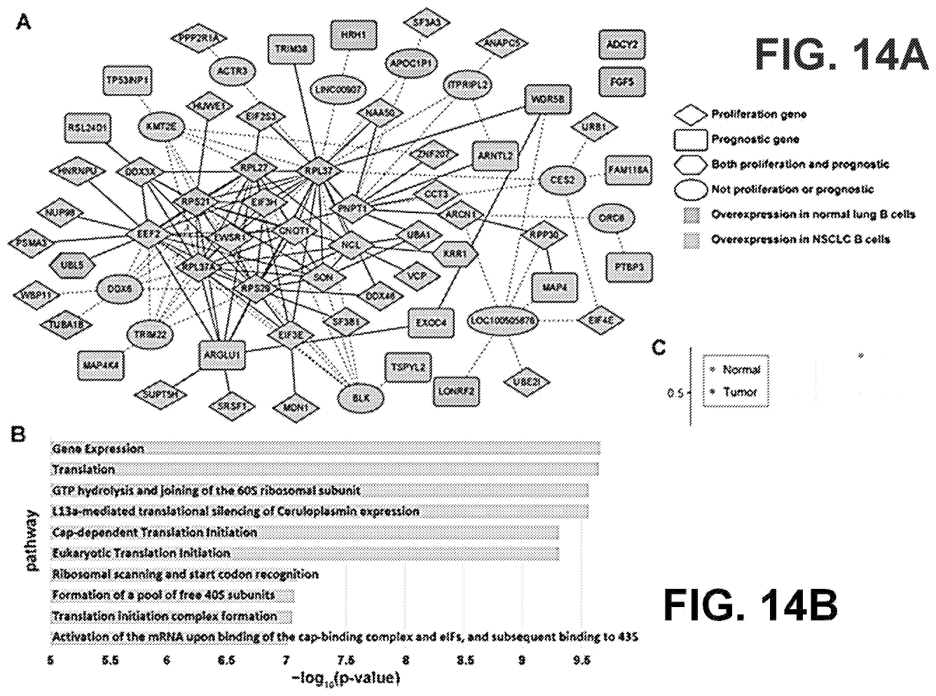

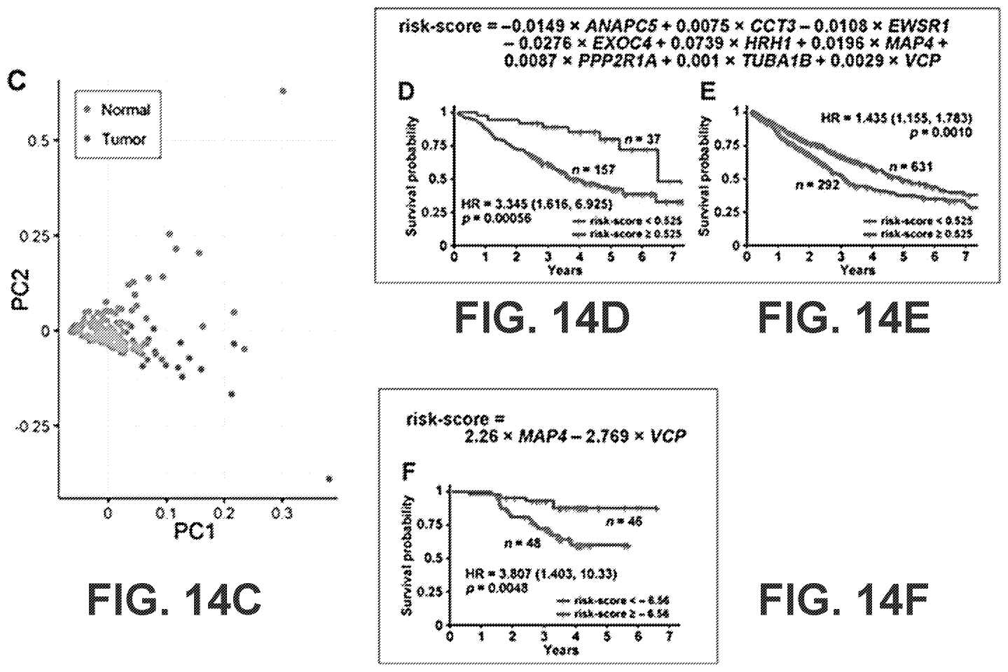

FIG. 8D shows that, among the genes identified in the multi-omics network (FIG. 8C), SERPINB5, TTC16, and CD27 had a significant differential expression (p<0.05, two sample t-tests) in the short survival (<20 mo.) versus long survival (>58 mo.) patient group (FIG. 14D). Many factors other than the response to radiotherapy may impact survival in this studied patient cohort. CD27 and TTC16 expressed higher in the short survival patient group, and SERPINB5 expressed higher in the long survival patient group. Down-regulation of SERPINB5 by small interfering RNA in NSCLC H460 cells increased the resistance of these cells to ionizing radiation, consistent with the observed association between SERPINB5 up-regulation and prolonged survival in TCGA NSCLC patients receiving radiotherapy.

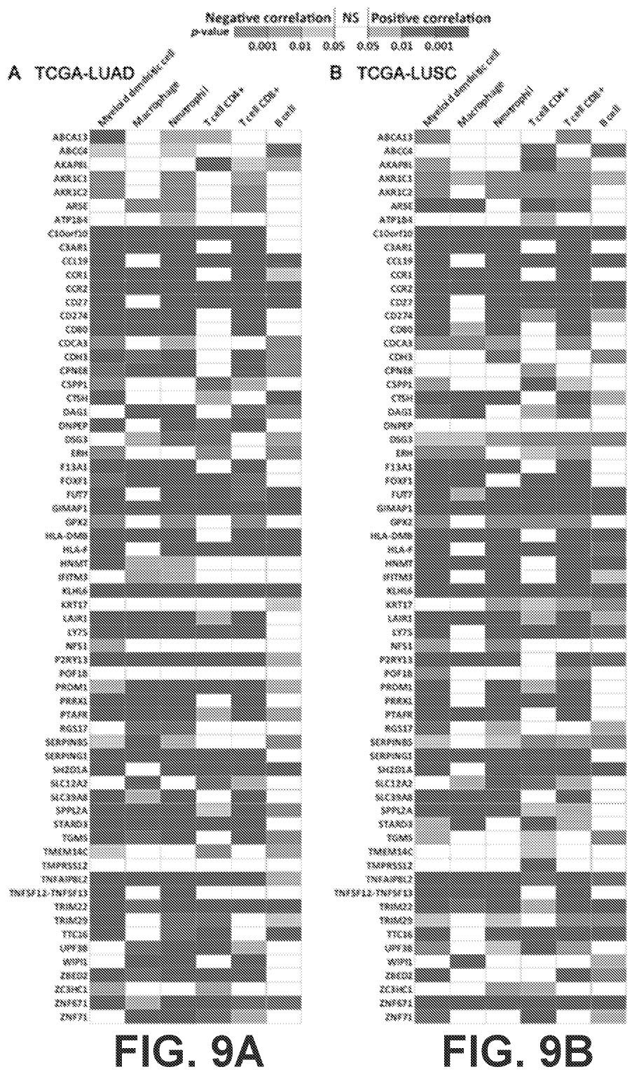

FIGS. 9A-D illustrate examples of an association between immune infiltration and expression of the multi-omics network genes, in accordance with various embodiments of the present disclosure. FIG. 9A shows the correlation of immune infiltration level with gene expression in TCGA-LUAD patients (n=515) estimated with TIMER 2.0. FIG. 9B shows the correlation of immune infiltration level with gene expression in TCGA-LUSC patients (n=501) estimated with TIMER 2.0. The immune infiltration analysis included myeloid dendritic cells, macrophages, neutrophils, CD4+ T cells, CD8+ T cells, and B cells. All the available genes in the 7-gene network had a significant correlation with immune infiltration in at least one examined cell types.

Furthermore, drug responses of 10 commonly used therapeutic regimens for treating NSCLC were included in this example, including carboplatin, cisplatin, paclitaxel (Taxol), pemetrexed, docetaxel, gemcitabine, vinorelbine, etoposide, gefitinib, and erlotinib. The NSCLC cell lines in this example include adenocarcinoma, squamous cell carcinoma, large cell carcinoma, and adenosquamous carcinoma. Due to the synergism and successful results of the combination of cisplatin-etoposide in treating small cell lung cancer, long-term daily administration of oral etoposide in combination with cisplatin was used to treat NSCLC. Cisplatin-etoposide has comparable efficacy as carboplatin-paclitaxel when used with concurrent radiotherapy for patients with stage III unresectable NSCLC. Paclitaxel, a tubulin-binding agent, can be used to treat NSCLC in combination with a platinum-based compound. Gefitinib and erlotinib can be used epidermal growth factor receptor (EGFR) tyrosine kinase inhibitors for treating advanced NSCLC with proven efficacy. Gefitinib and erlotinib have comparable effects on patient survival, overall response rate, and disease control rate, with no considerable variation associated with EGFR mutation status, ethnicity, line of treatment, and baseline brain metastasis status.

Docetaxel offers clinical benefits as a second-line treatment of NSCLC in patients previously treated with platinum-based chemotherapy. The combination of pembrolizumab (anti-PD1 immunotherapy) plus docetaxel was well tolerated and substantially improved progression-free survival and overall response rate in patients with advanced NSCLC after platinum-based chemotherapy, including patients with EGFR variations.

Here, genes associated with drug response were selected based on their differential mRNA expression and protein expression, respectively, in sensitive versus resistant NSCLC cell lines to the studied drugs. In the identified 7-gene network shown in FIG. 8C, multiple genes were associated with drug sensitivity or drug resistance to carboplatin, cisplatin, docetaxel, erlotinib, etoposide, gefitinib, gemcitabine, paclitaxel, pemetrexed, and vinorelbine in the CCLE NSCLC cell lines (Table 2-3).

TABLE 2
Genes (shown in FIG. 8C) with a significant differential mRNA expression (p < 0.05; two sample t-tests) and a fold change <
0.5 or > 2 in sensitive versus resistant CCLE NSCLC cell lines (n = 117) to specific drugs. BOLD font indicates higher mRNA experssion
in sensitive cell lines, and REGULAR indicates high mRNA epxression in resistant cell lines.

| | PRISM | GDSC1 | GDSC2 |
|---|---|---|---|
| Carboplatin | IFITM3, ATP1B4, PRRX1, PRRX1 | | |
| Cisplatin | FUT7 | | POF1B, ZNF671, ZBED2, C10orf10 |
| Docetaxel | ERH, KLHL6 | KLHL6, IFITM3, C10orf10 | SERPING1 |
| Erlotinib | KLHL6, TRIM29, IFITM3, CCR2, | IFITM3, SERPINB5, PRDM1, | DEFB103A, CSPP1, TRIM29, SERPINB5, |
| | TNFSF12-TNFSF13, ATP1B4 | RGS17, CD80, AKR1C1, TRIM29 | AKR1C2, KLHL6, ATP1B4, SERPING1, DSG3 |
| Etoposide | PRDM1, TRIM29 | ATP1B4, SERPING1, ABCA13, ARSE | |
| Gefitinib | DEFB103A, PTAFR, ZEB1, | DEFB103A, KLHL6, TRIM29, | CSPP1, KLHL6, TRIM29, ATP1B4, |
| | ERH, F13A1 | RGS17, ATP1B4, PRRX1 | SERPING1, ARSE |
| Gemcitabine | ARSE, GPX2, RGS17, LAIR1, | SERPING1 | ARSE, C10orf10 |
| | TRIM22 | | |
| Paclitaxel | PTAFR, TRIM29, RAB25, ESRP1 | CD27 | C10orf10 |
| | SERPINB5, KLHL6, CD80, SH2D1A | | |
| Pemetrexed | | SH2D1A | |
| Vinorelbine | FUT7, TNFSF12-TNFSF13, DSG3 | | |

TABLE 3
Genes (shown in FIG. 8C) with a significant differential protein expression (p < 0.05; two sample t-tests) and a fold change <
0.5 or > 2 in sensitive versus resistant CCLE NSCLC cell lines (n = 63) to specific drugs. BOLD font indicates higher protein experssion
in sensitive cell lines, and REGULAR font indicates higher protein epxression in resistant cell lines.

| | PRISM | GDSC1 | GDSC2 |
|---|---|---|---|
| Carboplatin | TGM5 | | |
| Cisplatin | TRIM29 | CD274, HLA-F | LY75, PRRX1, F13A1, CTSH |
| Docetaxel | SERPINB5 | AKR1C2, SERPINB5 | AKR1C2, AKR1C1, CPNE8 |
| Erlotinib | ARSE, HLA-DMB, TRIM22, LY75, TGM5 | AKR1C1 | POF1B, SERPINB5, F13A1 |
| Etoposide | HLA-F, ABCA13, TGM5 | SERPINB5 | |
| Gefitinib | | SLC39A8 | |
| Gemcitabine | TGM5, HNMT, CTSH, HLA-F, SERPINB5, CDH3, | SERPINB5, AKR1C1 | AKR1C1 |
| | HNMT, ABCA13, AKR1C2, GPX2, AKR1C1 | | |
| Paclitaxel | SERPINB5, CDH3, HLA-F, SLC39A8 | | PRRX1, LY75, CTSH, TRIM29 |
| Pemetrexed | | | |
| Vinorelbine | | SERPINB5, AKR1C2, | |
| | | AKR1C1, F13A1, HLA-F | |

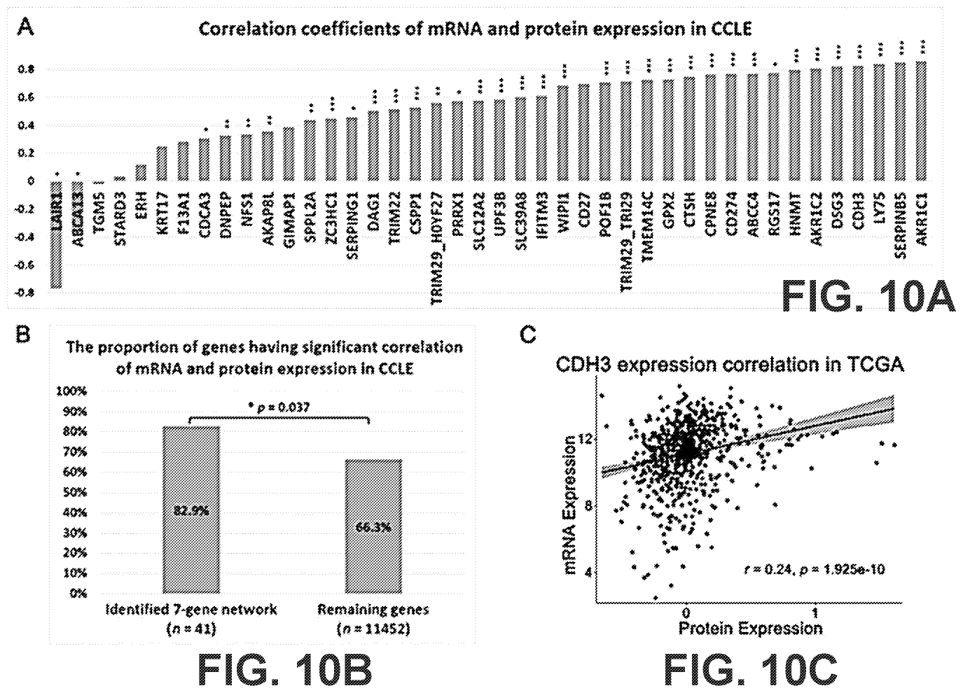

FIGS. 10A-C illustrate examples of the correlation between mRNA and protein expression of genes in the multi-omics network shown in FIG. 8C in NSCLC cell lines and patient tumors. FIG. 10A includes a bar chart that shows the correlation coefficients of mRNA and protein expression from the same gene in NSCLC cell lines. The asterisk (*) indicates the significance level (*p<0.05, **p<0.01, ***p<0.001). FIG. 10B shows the proportion of genes having a significant correlation of mRNA and protein expression in NSCLC cell lines (p<0.05, Pearson's correlation). The proportion of genes having a significant correlation between mRNA and protein expression is significantly higher in identified 7-gene network than the remaining genes in the genome (two-tailed proportion z-test, p<0.05). FIG. 10C shows that CDH3 had a significant positive correlation between mRNA and protein expression in combined TCGA-LUSC and TCGA-LUAD patient tumors.

In CCLE NSCLC cell lines (n=63), 41 genes in the 7-gene network had both mRNA and protein expression measurements. A total of 34 out of 41 genes had significant correlation between mRNA expression and protein expression in NSCLC cell lines (p<0.05, Pearson's correlation; FIG. 10A). TRIM29 had two corresponding protein expression quantifications, both significantly correlated with its mRNA expression. The proportion of genes having a significant mRNA and protein expression correlation is 82.9% in the identified 7-gene network, which is significantly higher than 66.3% in the remaining 11,452 genes in the genome (two-tailed proportion z-test p<0.05, FIG. 10B). In TCGA-LUAD and TCGA-LUSC tumors, only CDH3 and CD274 had both mRNA expression and protein expression. The protein expression of CDH3 was positively correlated with its mRNA expression in TCGA NSCLC patient tumors (p<0.05, Pearson's correlation; FIG. 10B), consistent with the observation in CCLE NSCLC cell lines (FIG. 10A).

Among this 7-gene signature, mRNA expression of ZNF71 was positively associated with survival in patients who received cisplatin, carboplatin, and Taxol in the studied cohorts, indicating its association with chemosensitivity. Although ZNF71 mRNA expression was not associated with NSCLC survival in the overall patient cohorts analyzed in qRT-PCR, a higher ZNF71 protein expression quantified with AQUA was associated with a more favorable survival outcome in two separate NSCLC cohorts (n=291) using Tissue Microarrays.

Protein level of overexpressed ZNF71 (GeneTex, Cat. No. GTX116553) was detected. ZNF71 gene is comprised of four exons, where exons 1 and 2 code for 180nt 5′UTR and the first 11 amino acids of the protein. Exon 3 codes for the next 43 amino acids (aa), encompassing most of the KRAB repression domain. Exon 4, the longest, codes for the remainder of the protein, including the 13-zinc-finger putative DNA binding domain and the predicted ˜4 kb 3′UTR. The third KRAB-domain-containing exon could be alternatively spliced out to produce a KRAB-less isoform that will produce a shorter in-frame protein encoded by the last exon, i.e., exon 4. Approximately half of 800 human C2H2-type ZNF genes contain the evolutionary conserved one-exon-encoded KRAB domain, which could be alternatively spiced. ZNF71 KRAB expression was associated with EMT in both NSCLC patient tumors and cell lines. ZNF71 protein expression was not available in the CCLE database.

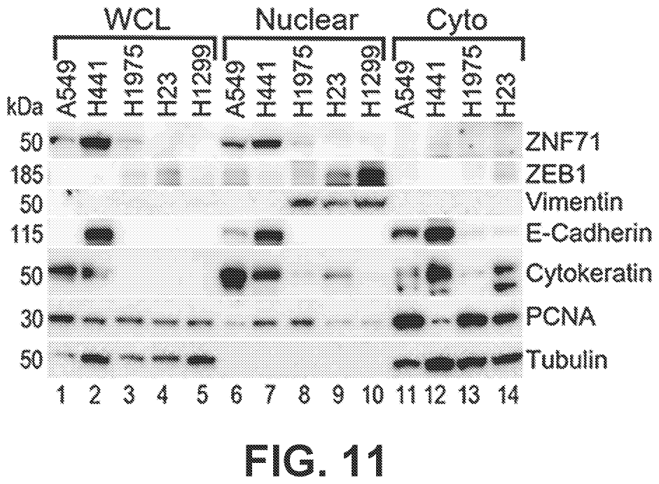

FIG. 11 illustrates an example of a correlation of expression of ZNF71 with EMT markers in five NSCLC cell lines. Correlation of expression of ZNF71 with EMT markers in five NSCLC cell lines. Western blotting analysis of ZNF71, mesenchymal and epithelial markers in the indicated cell lines. PCNA and Tubulin—loading controls. WCL—whole cell lysate, Nuclear and Cyto—nuclear and cytoplasmic fractions isolated from the same cell lines.

In the example of FIG. 11, ZNF71 protein expression (GeneTex, Cat. No. GTX116553) was detected in A549 and H441 cell lines in whole cell lysate and nuclear fractions isolated from the same cell lines (FIG. 11), consistent with its function as a transcription factor. ZNF71 protein expression was positively correlated with epithelial markers, including E-cadherin and Cytokeratin, and was negatively correlated with mesenchymal markers, including ZEB1 and Vimentin in Western blots. These results are consistent with the observed positive association between ZNF71 protein expression and prolonged NSCLC patient survival.

The expression of ZNF71 overall and isoforms was analyzed in the NGS dataset GSE81089 of NSCLC tumor samples (n=197). Proliferation networks of ZNF71, ZNF71 KRAB, and ZNF71 KRAB-less were constructed, respectively, with the Boolean implication network algorithm. First, genes that had a significant (p<0.05, z-tests) co-expression with ZNF71, ZNF71 KRAB, or ZNF71 KRAB-less in GSE81089 were identified in the genome-scale analysis, respectively. Next, the proliferation genes which meet the criteria were selected based on CRISPR-Cas9/RNAi screening data in CCLE NSCLC cell lines. These proliferation genes were pinpointed in the gene co-expression networks to form the proliferation networks.

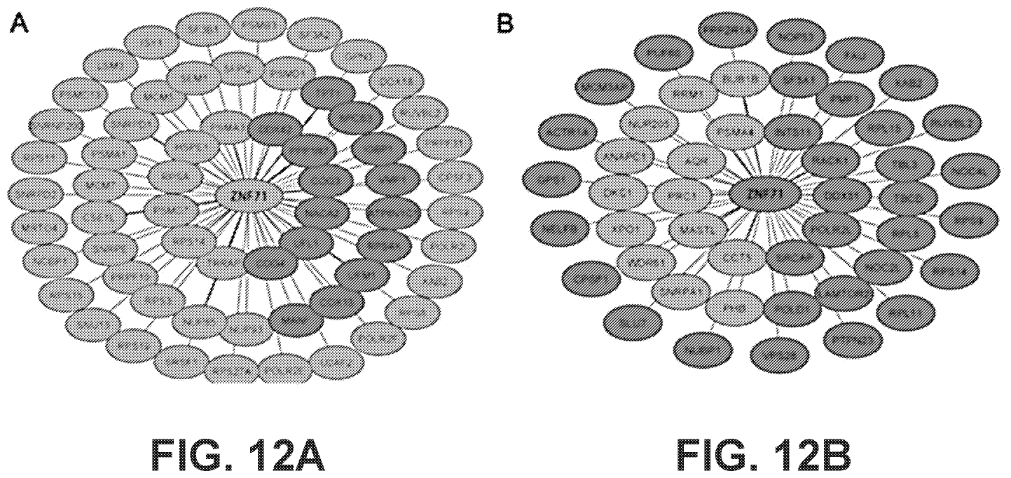

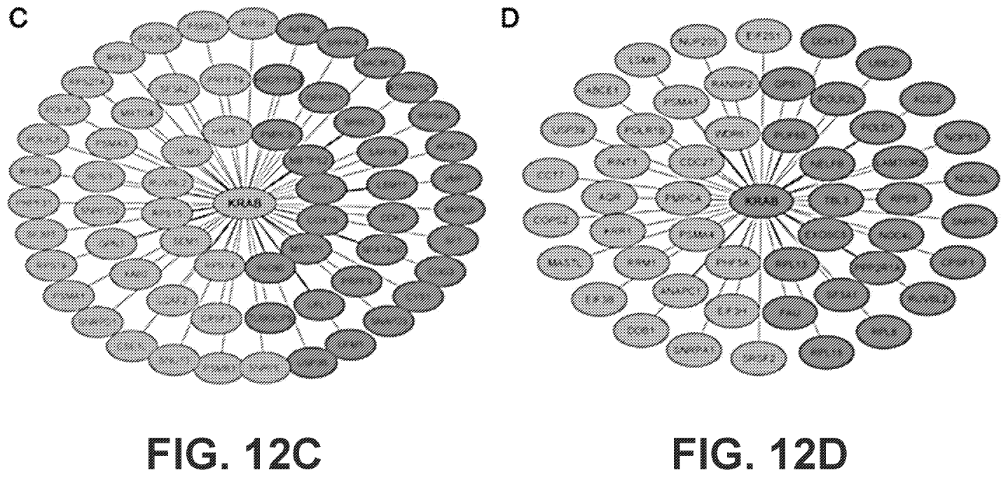

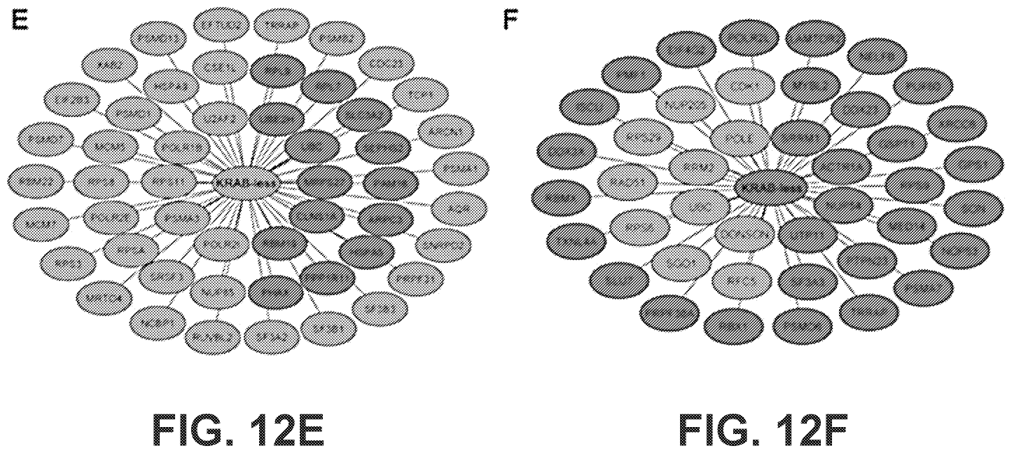

FIGS. 12A-F illustrate examples of ZNF71 gene level, ZNF71 KRAB, and ZNF71 KRAB-less co-expression networks in patient cohort GSE81089. FIGS. 12A-F show several categories of the constructed proliferation networks. FIG. 12A shows proliferation genes that had a significant (p<0.05, z-tests) co-expression with up-regulated ZNF71. FIG. 12B shows proliferation genes that had a significant (p<0.05, z-tests) co-expression with down-regulated ZNF71. FIG. 12C shows proliferation genes that had a significant (p<0.05, z-tests) co-expression with up-regulated ZNF71 KRAB isoform. FIG. 12D shows proliferation genes that had a significant (p<0.05, z-tests) co-expression with down-regulated ZNF71 KRAB isoform. FIG. 12E shows proliferation genes that had a significant (p<0.05, z-tests) co-expression with up-regulated ZNF71 KRAB-less isoform. FIG. 12F shows proliferation genes that had a significant (p<0.05, z-tests) co-expression with down-regulated ZNF71 KRAB-less isoform. The gene expression signatures containing up-and down-regulated NSCLC proliferation genes from these 6 networks were input into CMap, respectively, to identify significant (p<0.05) perturbagen signatures and functional pathways. The common significant (connectivity score>0.9) functional pathways were summarized for ZNF71 (Table 4), ZNF71 KRAB (Table 5), and ZNF71 KRAB-less (Table 6).

The functional pathways associated with the identified proliferation networks of ZNF71, KRAB, and KRAB-less isoforms were largely different, although with some overlaps, i.e., CP_FGFR_INHIBITOR between ZNF71 and KRAB, and PID_CIRCADIAN_PATHWAY for ZNF71 and KEGG_CIRCADIAN_RHYTHM_MAMMAL for KRAB-less. When the up-regulated and down-regulated proliferation genes in each network (FIG. 12) were uploaded to CMap separately, significant (p<0.05, connectivity score>0.9) perturbagen signatures and functional pathways were identified, showing that KD_AHSP_PATHWAY, KD_PHOSPHOLIPASES, and KD_PI3K_SIGNALING were commonly associated with ZNF71, KRAB, and KRAB-less proliferation networks. These results generated hypotheses for future mechanistic studies of ZNF71 and its isoforms.

TABLE 4
The common significant (p < 0.05, connectivity score > 0.9) functional pathways from CMap using ZNF71 up-regulated
network genes (FIG. 12A) and ZNF71 down-regulated network genes (FIG. 12B) as input.

| | ZNF71 upregulated | | ZNF71 downregulated | |
|---|---|---|---|---|
| | network genes | | network genes | |
| src_set_id | cell line | type | cell line | type |
| BIOCARTA_CTL_PATHWAY | A549 | TRT_SH.CGS | A549 | TRT_SH.CGS |
| | HCC515 | TRT_SH.CGS | HCC515 | TRT_SH.CGS |
| CP_FGFR_INHIBITOR | A549 | TRT_CP | A549 | TRT_CP |
| (PD-173074, dovitinib, brivanib, orantinib) | | | | |
| KD_RNA_POLYMERASE_ENZYMES | A549 | TRT_SH.CGS | HCC515 | TRT_SH.CGS |
| (POLR2A, POLR2C, POLR2D, POLR2E, POLR2F, POLR21, POLR2K) | | | | |
| KEGG_GALACTOSE_METABOLISM | HCC515 | TRT_SH.CGS | HCC515 | TRT_SH.CGS |
| PID_CIRCADIAN_PATHWAY | A549 | TRT_SH.CGS | A549 | TRT_SH.CGS |
| PID_INTEGRIN2_PATHWAY | HCC515 | TRT_SH.CGS | A549 | TRT_SH.CGS |
| PID_S1P_S1P2_PATHWAY | HCC515 | TRT_SH.CGS | HCC515 | TRT_SH.CGS |
| REACTOME_CHYLOMICRON_MEDIATED_LIPID_TRANSPORT | HCC515 | TRT_SH.CGS | HCC515 | TRT_SH.CGS |
| REACTOME_JNK_C_JUN_KINASES_PHOSPHORYLATION_AND_ | HCC515 | TRT_SH.CGS | HCC515 | TRT_SH.CGS |
| ACTIVATION_MEDIATED_BY_ACTIVATED_HUMAN_TAK1 | | | | |
| REACTOME_PURINE_RIBONUCLEOSIDE_ | A549 | TRT_SH.CGS | A549 | TRT_SH.CGS |
| MONOPHOSPHATE_BIOSYNTHESIS | | | HCC515 | TRT_SH.CGS |
| SULFONYLUREA | HCC515 | TRT_CP | HCC515 | TRT_CP |
| (glipizide, glibenclamide, gliquidone) | | | | |

TABLE 5
The common significant (p < 0.05, connectivity score > 0.9) fucntional pathways from CMap using ZNF71 KRAB upregulated
network genes (FIG. 12C) and ZNF71 KRAB downregulated network genes (FIG. 12D) as input.

| | KRAB upregulated | | KRAB downregulated | |
|---|---|---|---|---|
| | network genes | | network genes | |
| src_set_id | cell line | type | cell line | type |
| BIOCARTA_CTL_PATHWAY | HCC515 | TRT_SH.CGS | HCC515 | TRT_SH.CGS |
| | | | A549 | TRT_SH.CGS |
| BIOCARTA_GLYCOLYSIS_PATHWAY | HCC515 | TRT_SH.CGS | A549 | TRT_SH.CGS |
| BIOCARTA_SET_PATHWAY | A549 | TRT_OE | A549 | TRT_OE |
| CP_CCK_RECEPTOR_ANTAGONIST | HCC515 | TRT_CP | HCC515 | TRT_CP |
| (devazepide, LY-225910, SR-27897) | | | | |
| CP_FGFR_INHIBITOR | A549 | TRT_CP | HCC515 | TRT_CP |
| (PD-173074, dovitinib, brivanib, orantinib) | | | | |
| KD_INTEGRIN_SUBUNITS_BETA | A549 | TRT_XPR | A549 | TRT_XPR |
| (ITGB1, ITGB4, ITGB5) | | | | |
| PID_DNA_PK_PATHWAY | A549 | TRT_SH.CGS | A549 | TRT_SH.CGS |
| REACTOME NFKB_IS_ACTIVATED_AND_SIGNALS_SURVIVAL | A549 | TRT_SH.CGS | HCC515 | TRT_SH.CGS |
| REACTOME_REGULATION_OF_COMPLEMENT_CASCADE | HCC515 | TRT_SH.CGS | HCC515 | TRT_SH.CGS |
| SODIUM/GLUCOSE_COTRANSPORTER_INHIBITOR | A549 | TRT_CP | A549 | TRT_CP |
| (maackiain, phloretin) | | | | |
| SULFONYLUREA | HCC515 | TRT_CP | HCC515 | TRT_CP |
| (glipizide, glibenclamide, gliquidone) | | | | |
| TGF_BETA_RECEPTOR_INHIBITOR | HCC515 | TRT_CP | HCC515 | TRT_CP |

TABLE 6
The common significant (p < 0.05, connectivity score > 0.9) functional pathways from CMap using ZNF71 KRAB-less
upregulated network genes (FIG. 12E) and ZNF71 KRAB-less downregulated network genes (FIG. 12F) as input.

| | KRAB-less upregulated | | KRAB-less downregulated | |
|---|---|---|---|---|
| | network genes | | network genes | |
| src_set_id | cell line | type | cell line | type |
| ABL_KINASE_INHIBITOR | A549 | TRT_CP | HCC515 | TRT_CP |
| BIOCARTA_BLYMPHOCYTE_PATHWAY | A549 | TRT_SH.CGS | A549 | TRT_SH.CGS |
| KD_CYCLINS | HCC515 | TRT_SH.CGS | A549 | TRT_OE |
| (CCNL1, CCND1, CCNA1, CCNH) | | | | |
| KEGG_CIRCADIAN_RHYTHM_MAMMAL | A549 | TRT_SH.CGS | A549 | TRT_SH.CGS |
| REACTOME_TRAF3_DEPENDENT_IRF_ | A549 | TRT_SH.CGS | A549 | TRT_SH.CGS |
| ACTIVATION_PATHWAY | | | | |

Functional Pathways Associated with the 7-Gene Multi-Omics Network and Discovery of Repositioning Drugs

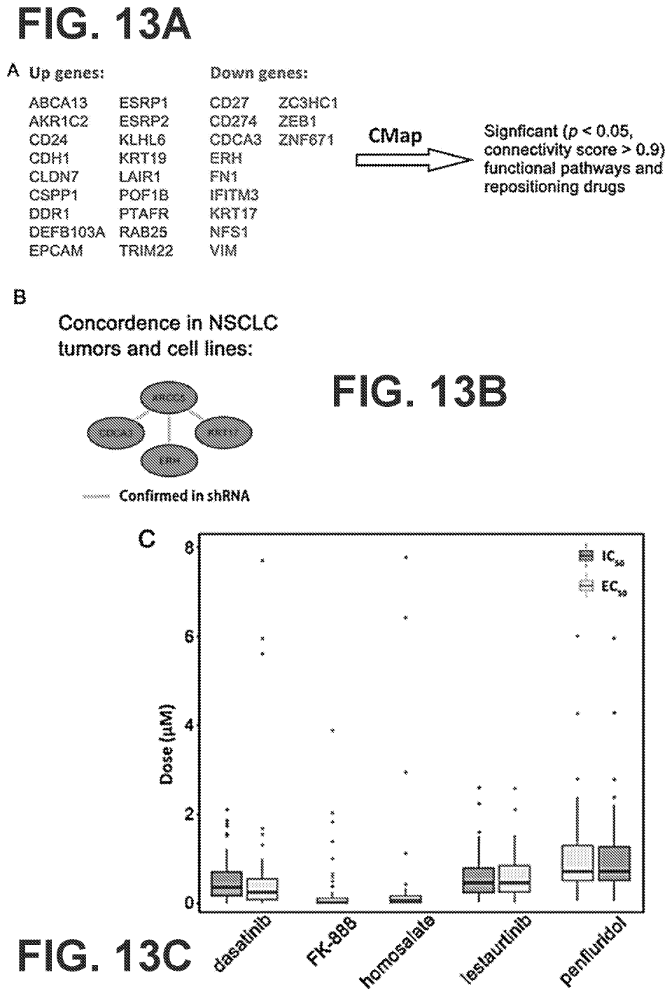

FIGS. 13A-C illustrate examples of identification of functional pathways associated with the 7-gene multi-omics network and discovery of repostitioning drugs. ZNF71 KRAB was reported to be associated with EMT. Protein expression of ZNF71 was positively correlated with epithelial markers and negatively correlated with mesenchymal markers in the nuclear fraction of NSCLC cells (FIG. 11). The identified 7-gene multi-omics network (FIG. 8C) and 14 EMT markers were used to discover connected perturbagen signatures and repositioning drugs with potential implications in improving NSCLC treatment.

The following mechanisms of action, for example, were considered to reverse EMT, to enhance drug sensitivity, to induce immune response, and to inhibit NSCLC proliferation through 1) up-regulation of epithelial markers; 2) up-regulation of drug sensitive genes; 3) down-regulation of mesenchymal markers; 4) down-regulation of proliferation genes; and 5) down-regulation of CD27 and PD-L1 (CD274). The 14-gene EMT classifier was used to define the EMT states of NSCLC tumors, including 3 mesenchymal markers (ZEB1, VIM, and FN1) and 11 epithelial markers (CDH1, EPCAM, ESRP1, ESRP2, DDR1, CTNNB1, CD24, CLDN7, KRT8, KRT19, and RAB25). The drug sensitive genes were selected if a gene's mRNA expression is sensitive to one or more of the studied 10 NSCLC therapeutic regimens and is not resistant to any studied drugs in PRISM or GDSC1/2 datasets. The drug sensitive genes and the epithelial markers were excluded from the list if they were also proliferation genes. Here, the proliferation genes referred to those with a significant dependency score (<−0.5) in CRISPR-Cas9 or RNAi in more than 10 NSCLC cell lines. The proliferation gene list also excluded the epithelial markers (FIG. 13A).

FIG. 13A shows selection of significant functional pathways and repositioning drugs based on the identified 7-gene multi-omics prolieration network with CMap.

FIG. 13B shows confirmed co-expression network of XRCC5 in NSCLC patient tumors in GSE31800 or GSE28582 showing concordant co-expression patterns after shRNA knock-down of XRCC5 in A549 cell line. The single line indicates down-regulation or non-upregulation of genes. The double line indicates that the co-expression relations (p<0.05, z-tests) observed in patient tumors were confirmed in shRNA knockdown experiments in LINCS L1000 CMap NSCLC cell lines.

FIG. 13C shows small molecules that had a low average concentration of drug response in the CCLE NSCLC cell lines. Measurements with a drug activity value larger than 10 μM were considered as outliers and were removed from the plot. Dasatinib EC50 measurements had 2.38% of outliers. FK-888 EC50 measurements had 14.49% of outliers. Homosalate EC50 measurements had 7.25% of outliers. Penfluridol had 1.25% of outliers in both IC50 and EC50 measurements.

With the above defined up-and down-regulated gene lists, significant (p<0.05, connectivity score>0.9) functional pathways were identified with CMap (Table 6). Specifically, shRNA knock-down of the following pathways matched the 7-gene multi-omics network, including KD_APOLIPOPROTEINS, KD_CYCLINS, KD_LYSINE_ACETYLTRANSFERASES, KD_NON_HOMOLOGOUS_END_JOINING, KD_V_TYPE_ATPASES, and OE_NFKB_ACTIVATION (Table 6). After checking the gene co-expression networks in NSCLC patient tumors in GSE31800 and GSE28582, XRCC5 from KD_NON_HOMOLOGOUS_END_JOINING pathway was found to have the same gene co-expression patterns in patient tumors as in shRNA knock-down in A549 cell line (FIG. 13B; Table 7). A co-downregulation between XRCC5 and CDCA3, ERH, and KRT17, respectively, was found in both NSCLC tumors and LINCS L1000 CMap NSCLC cell lines (FIG. 13B). Low XRCC5 protein expression was more prevalent among squamous cell carcinoma compared with lung adenocarcinoma compared with normal lung tissues. Cell division cycle associated 3 gene (CDCA3) mRNA and protein expressions were increased in NSCLC versus in nonmalignant tissue, with high levels of CDCA3 being associated with poor patient prognosis.

TABLE 7
The identified significant (p < 0.05, connectivity score > 0.9) functional pathways from
shRNA know-down experiments in CMap using the up-regulated and down-regulated gene
lists in FIG. 13A as input.

| src_set_id | cell_iname | pert_type | genes |
|---|---|---|---|
| KD_APOLIPOPROTEINS | A549 | TRT_SH.CGS | APOB, APOC2, APOE |
| KD_CYCLINS | A549 | TRT_SH.CGS | CCNL1, CCND1, CCNA1, CCNH |
| | HCC515 | TRT_SH.CGS | |
| KD_LYSINE_ACETYLTRANSFERASES | A549 | TRT_SH.CGS | KAT6B, KAT6A, NCOA3 |
| KD_NON_HOMOLOGOUS_END_JOINING | A549 | TRT_SH.CGS | RAD50, FEN1, XRCC4, XRCC5 |
| KD_V_TYPE_ATPASES | HCC515 | TRT_SH.CGS | ATP6V1A, ATP6V0C, ATP6VOB, |
| | | | ATP6V1F |
| OE_NFKB_ACTIVATION | HCC515 | TRT_SH.CGS | CD40, FADD, LTBR, TNFRSF10A, |
| | | | TNFRSF10B, TNFRSF1A, BCL10 |

Based on the designed mechanisms of action, 39 repositioning drugs were identified with CMap (p<0.05). From this list, drugs that have lower average IC₅₀ and EC₅₀ in CCLE NSCLC cell lines were selected for potential inhibition of the growth of lung cancer cells with a safer dose. In the original PRISM drug screen, eight doses ranging from 0.0006 μM to 10 μM were tested on most of the compounds. Five small molecules (dasatinib, FK-888, homosalate, lestaurtinib, and penfluridol) had a small average IC₅₀ or EC₅₀ value (smaller than or close to 1 μM) in the PRISM drug screen and were selected for drug repurposing (FIG. 13C). Dasatinib is a targeted therapy used to treat chronic myelogenous leukemia and acute lymphoblastic leukemia. FK 888 is a selective, high-affinity tachykinin NK1 receptor antagonist. Homosalate is an organic compound commonly used in sunscreen that absorbs shortwave UVB rays, which helps preventing DNA damage and decreasing the risk of skin cancer. Lestaurtinib is a tyrosine kinase inhibitor and is structurally related to staurosporine. It has undergone clinical trials for the treatment of various cancers, including leukemia. and refractory pediatric neuroblastoma. Penfluridol is the first-generation antipsychotic drug used to treat schizophrenia. These results provided evidence for future drug repurposing study to improve NSCLC treatment in combination with clinical therapies.

### Discussion

Lung cancer remains the leading cause of cancer-related mortality in both men and women due to its complex etiology, advanced disease stage at the time of diagnosis, and a lack of biomarkers for the selection of chemotherapy, immunotherapy and radiotherapy in individual patients based on their tumor characteristics. According to the National Cancer Institute, 56% of lung cancer cases are at the distant stage, meaning that cancer has metastasized at the time of diagnosis. Novel biomarkers can aid the selection of appropriate chemotherapy and immunotherapy for treatment of advanced NSCLC. For early-stage NSCLC, it remains a challenge to cure the disease and improve survival outcomes due to the varying performance of lymphadenectomy, the limitations of current pathologic nodal staging, and insufficient understanding of molecular features underlying tumor aggressiveness. The development of clinically applicable prognostic biomarkers for early-stage NSCLC could potentially resolve this issue by identifying more aggressive tumors prone to recurrence/metastasis beyond the morphological assessment. Patients with molecularly more aggressive tumors might benefit from adjuvant therapy. To date, physicians do not have a precise tool to identify patients with resectable NSCLC who are likely to develop recurrence or to predict clinical benefits of chemotherapy followed by immunotherapy. Thus, a significant unmet need exists for a reliable diagnostic method to 1) identify stage I NSCLC who might benefit from adjuvant chemotherapy; and 2) select specific therapeutic regimens that a resectable NSCLC patient might respond to.

These needs are met with the development of a qRT-PCR based 7-gene microfluidic assay for resectable NSCLC prognosis and prediction of benefits of chemotherapy. The 7-gene assay was developed from the training cohort (n=83) with accurate patient stratification (p=0.0043) and was validated in independent patient cohorts including a clinical trial JBR.10. (n=248, p<0.0001) in Kaplan-Meier analysis. In the predicted chemotherapy-benefit group, there was a significantly better disease-specific survival in patients receiving adjuvant chemotherapy in both training (p=0.035) and validation (p=0.0049) sets. In the predicted non-benefit group, there was no survival benefit in patients receiving chemotherapy in either set. The ability of this gene assay to inform selection of specific adjuvant chemotherapy could potentially improve patient care. The present disclosure shows that is feasible to apply this 7-gene signature for NSCLC prognosis using NGS profiles of the TCGA patient tumors randomly partitioned into training and testing sets. In the future studies, the development of an NGS-based 7-gene assay will need to be defined with specific NGS data generation protocols, data processing methods, and model parameters from the training set that are validated in external patient cohorts for potential clinical applications.

Within this 7-gene assay, ZNF71 KRAB isoform is associated with EMT in NSCLC tumors and cell lines, i.e., expressed higher in top 50% of ZEB1 expressing cells. ZNF71 protein expression was positively correlated with epithelial markers, including E-cadherin and Cytokeratin, and was negatively correlated with mesenchymal markers, including ZEB1 and Vimentin in NSCLC cell lines in Western blots. These results were consistent with the observed positive association between ZNF71 protein expression and prolonged NSCLC patient survival. Together, these results supported the transcriptional repression role of the KRAB domain.

Zinc finger proteins (ZFPs) are the largest family of transcription factors in human cells and in most eukaryotes, representing about 3% of the total human genome. A repeating motif within ZFPs contains two histidine and two cysteine amino acid residues (i.e., C2H2) coordinating zinc ion, which has the ability to bind to DNA, RNA, or cellular proteins. ZFPs thus have a wide range of predicted functions according to their molecular structure, including DNA repair, degradation of proteins, signal transductions, migration of cells, regulation of apoptosis, lipid binding, and transcription regulation. KRAB-ZFPs are a family of transcriptional repressors with diverse functions, most notably the silencing of transposable elements. They contain an N-terminal KRAB domain and a C-terminal C2H2-type zinc finger array. The repressor action of KRAB-ZFPs requires the recruitment of KRAB Associated Protein 1, also known as Tripartite Motif Protein 28, KAP1/TRIM28. KAP1 functions as a scaffold complex composed of histone methyl transferase (SETDB1), Heterochromatin Protein-1 (HP-1), Nucleosome Remodeling and Deacetylation (NuRD), and DNA Methyl Transferase. When KAP-1 is recruited to KRAB-ZFPs, the formed repressor complex leads to heterochromatin formation. KAP1 promotes proliferation and metastatic progression of breast cancer cells, consistent with the observed association between KAP1 and breast cancer progression. RB-associated KRAB zinc finger (BRAK) was upregulated in NSCLC and was associated with patient poor-prognosis. Zinc finger protein 668 (ZNF668) was reported to suppress NSCLC invasion and migration by down-regulating Snail and upregulating E-cadherin and zonula occludens-1. Down-regulated protein expression of ZNF668 was found in NSCLC tumors compared with normal lung tissues, and there was a negative association between ZNF668 protein expression and lymph node metastasis.

A mechanistic link between ZNF71 KRAB, ZNF71 protein, and EMT is currently not known. EMT could be associated with significant changes in splicing and hence can potentially skew splicing for inclusion of the KRAB domain. The link between other KRAB-ZFPs and EMT was reported. TRIM28 protein, a universal co-factor for KRAB-ZFP transcription factors, is known to participate in a wide range of aspects of cellular biology, either promoting cell proliferation. or mediating anti-proliferative activities. TRIM28 protein is involved in cancer by regulating gene expression through heterochromatin formation, mediation of DNA damage response, inhibition of p53 activity, regulation of EMT, and maintenance of stem cell pluripotency and genome stability. TRIM28 expression is induced following TGF-β treatment at both protein and mRNA levels. TRIM28 deficiency impairs TGF-β induced EMT and decreases cell migration and invasion, and the expression of TRIM28 affects the acetylation of histones on E-cadherin and N-cadherin promoters, suggesting that TRIM28 contributes to EMT and might be relevant for tumor metastasis in lung cancer. To gain a better understanding of ZNF71 and its isoforms, genome-scale co-expression networks containing all the genes with a statistically significant co-expression association relations with ZNF71 overall, KRAB, and KRAB-less were constructed, respectively. Proliferation networks were dissected based on CRISPR-Cas9/RNAi screening data in CCLE NSCLC panel. Significant functional pathways matching the up-and down-regulation signatures of these proliferation networks were identified with L1000 CMap. The results showed that multiple different functional pathways were associated with ZNF71, KRAB, and KRAB-less isoforms in shRNA knock-down or overexpression assays in NSCLC cell lines, respectively. Three pathways, including KD_AHSP_PATHWAY, KD_PHOSPHOLIPASES, and KD_PI3K_SIGNALING in shRNA knock-down assays of NSCLC cell liens, were commonly associated with ZNF71, KRAB, and KRAB-less isoforms. These results provided hypotheses of future molecular and cellular studies of ZNF71 and its isoforms in NSCLC.

The identified 7 signature genes have interactions with major inflammatory and cancer signaling hallmarks including TNF, PI3K, NF-kB, and TGF-β. The protein expression of CD27 quantified with ELISA had a strong correlation with its mRNA expression in NSCLC tumors. CD27, a new generation of ICI, is being tested as adjuvant therapy in phase I/II clinical trials for multiple tumor types with promising results. Increasing evidence supports that CD27 agonist antibodies, either alone or combined with PD1-blockade, can improve the therapeutic efficacy of cancer vaccines and immunotherapy in general. The synergy between PD-1 blockade and CD27 stimulation for CD8+ T-cell driven anti-tumor immunity was reported recently, indicating the therapeutic potential of CD27 in neoadjuvant PD-1 blockade in resectable lung cancer.

Novel combinations of chemotherapy with immunotherapy complicate biomarker discovery and multiple complex interactions relevant to immune responses should be considered in this process. NSCLC tumor immune microenvironment is a complex molecular network functioning in multi-dimensional components of immune and stromal cells as well as blood vessels, exerting intricate interactions with epithelial tumor cells. To model this complicated molecular machinery, the Boolean implication network scheme was developed to integrate genome-scale CNV and gene expression profiles in NSCLC tumors. The multi-omics network involving the 7-gene signature was dissected. All the network genes, including PD-L1 (CD274), had a significant correlation with immune infiltration in at least one immune cell types of TCGA NSCLC tumors, including myeloid dendritic cells, macrophages, neutrophils, CD4+ T cells, CD8+ T cells, and B cells. The mRNA and protein expressions of numerous network genes were associated with drug sensitivity or resistance to 10 commonly used therapeutic regimens for treating NSCLC. The multi-omics network genes had a higher percentage (82.9%) of correlated mRNA and protein expression in CCLE NSCLC cell lines compared with the rest of the genome (66.3%). These results further substantiate the potential clinical implications of the multi-omics network of the 7-gene signature.

The presented Boolean implication network algorithm is conceptually innovative and has several advantages over other methodologies. 1) It can analyze discrete CNV data and continuous gene expression data in a seamless and biologically relevant manner that correlation networks cannot; 2) Unlike the acyclic Bayesian networks, the Boolean implication networks can model molecular circuits, such as feedback loops, which are relevant in biological switches and signaling motifs that regulate large-scale cellular processes; and 3) the Boolean implication networks can model multivariate data based on prediction logic in combination with robust statistical tests, whereas other Boolean networks are primarily focused on binary variables. This is an improvement because categorized CNV and gene expression data should have at least three states to be biologically relevant, representing amplification/normal/deletion, or upregulation/normal/downregulation.

Pathway and network approaches will lead to the discovery of novel therapeutic targets as well as repositioning drug candidates. Drug repositioning can improve patient care, due to a lower risk of failure, expediated drug development, and less investment. To design therapeutic strategies to reverse EMT, enhance drug sensitivity, and inhibit NSCLC proliferation, significant functional pathways and repositioning drugs were identified with L1000 CMap. The 14-gene EMT classifier to characterize NSCLC tumors, the multi-omics network of the 7-gene signature, and CRISPR-Cas9/RNAi screening data were used to generate up-and down-regulated gene lists as input to CMap. ShRNA Knock-down of the following pathways were linked to these mechanisms, including KD_APOLIPOPROTEINS, KD_CYCLINS, KD_LYSINE_ACETYLTRANSFERASES, KD_NON_HOMOLOGOUS_END_JOINING, KD_V_TYPE_ATPASES, and OE_NFKB_ACTIVATION. Nuclear factor kappa B (NF-κB) is a master regulator in NSCLC pathogenesis and progression. Activated NF-κB signaling may influence the progression of a lung tumor either positively via triggering immune surveillance. or negatively via promoting inflammation and tumorigenesis. Activation of NF-κB stimulates transcription of cyclin D1, a key regulator of G1 checkpoint control. NNK, a tobacco-specific nitrosamine, can stimulate proliferation through activation of the NF-κB and subsequent upregulation of cyclin D1 in a normal human bronchial cell. Inhibition of NF-κB results in loss of induction of EMT master-switch transcription factors in NSCLC. NF-κB activation has been associated with resistance to chemotherapeutics and tyrosine kinase inhibitors in EGFR mutant lung cancer. Protein expression of V-ATPase is associated with drug resistance and higher tumor grade in NSCLC. Dasatinib and lestaurtinib used for leukemia treatment were identified as repurposing drugs for NSCLC. Homosalate, FK 888, and penfluridol were discovered as potential repositioning drugs that can kill NSCLC cells, maintaining the expression of drug sensitivity genes and epithelial markers, and inhibit mesenchymal markers, proliferation genes, and ICIs for NSCLC treatment.

### Materials and Methods

### Patient Cohorts

TCGA NSCLC Patient Cohorts. The TCGA-LUAD (n=515) and TCGA-LUSC (n=501) data were available from LinkedOmics. (http://linkedomics.org/, accessed on 28 Apr. 2021). The gene expression was quantified with Illumina HiSeq 2000 RNA Sequencing platform (Illumina, San Diego, CA, USA). The RNA-Seq data values were log-transformed. Normalized gene-level reverse phase protein array data were also available from the source. The TCGA-LUSC and TCGA-LUAD dataset also had the cancer stage and radiotherapy information for each sample.

NSCLC Patient Cohort GSE31800. DNA copy number profiles of 271 NSCLC tumor samples were measured, including 179 adenocarcinoma samples and 92 squamous cell carcinoma samples (raw data available at NCBI GEO with accession number GSE31800). The DNA copy number profiles were measured with whole genome tiling path array CGH. A total of 49 out of the 271 samples had matched custom microarray gene expression profiles, with 29 adenocarcinomas and 20 squamous cell carcinomas.

NSCLC Patient Cohort GSE28582. A publicly available NSCLC patient cohort (n=100) was included in this study, including 50 adenocarcinomas, 22 large cell carcinomas, and 28 squamous cell carcinomas (raw data available at NCBI GEO with the accession number GSE28582). All the samples had matched DNA copy number variation profiled by SNP array and microarray gene expression data. Patients who survived shorter than 20 months after treatment were defined as short-term survivors, and those who survived longer than 58 months after treatment were defined as long-term survivors.

NSCLC Patient Cohort GSE81089. A total of 199 NSCLC patient tumors were collected. Deep RNA sequencing of the patient tumors was generated with Illumina HiSeq 2500 (raw data available at NCBI GEO with accession number GSE81089). Patients with sufficient survival information (n=197) were included in this study. The raw RNA-seq data were processed with Salmon to quantify the expression of ZNF71 at the isoform level by using transcripts per million (TPM) reads.

### Data Pre-Processing

CNV Data Pre-Processing. The original copy number data of GSE31800 was converted from human genome assembly Hg18 to Hg38 in this study. According to the converted chromosomal locations, 26,061 genes were matched. Bioconductor R package “CGHbase” (v1.46.0), and “CGHcall” (v2.48.0). were used to call the gains and losses of the copy number data

The original GSE28582 data were processed by converting the Hg17 reference to Hg38 in this study. According to the converted chromosomal locations, 12,842 genes were matched. PennCNV package. was used to process the SNP array data, the CNV data were categorized into 3 levels: 1 (amplification), −1 (deletion), and 0 (normal).

Gene Expression Data Pre-Processing. To define the level of gene expression, 27 housekeeping genes (ACTB, B2M, CDKN1B, ESD, FLOT2, GAPDH, GRB2, GUSB, HMBS, HPRT1, HSP90AB1, IPO8, LDHA, NONO, PGK1, POLR2A, PPIA, PPIH, PPP1CA, RHOA, RPL13A, SDCBP, TBP, TFRC, UBC, YAP1, YWHAZ) [10,95-98] were used to define the thresholds of gene expression categorization. The total percentage of over-expression and under-expression samples for all the housekeeping genes was fixed to be 30%, and the number n which meets the following Equation (1) was computed for each gene:

$$( the number of samples with expression > mean + n \times std ) + ( the number of samples with expression < mean - n \times std ) = 30 \% of the total number of samples \qquad ( 1 )$$

The averaged n value of all 27 housekeeping genes was 0.874 in the dataset GSE31800 and 0.977 in the dataset GSE28582. These n values were used to categorize each gene expression dataset. The mean and standard deviation for each gene were calculated. The sample with a gene expression higher than (mean+n×standard deviation) was defined as over-expressed; the sample with a gene expression lower than (mean−n×standard deviation) was defined as under-expressed; the sample with a gene expression in between the two thresholds was defined as normal. After categorizing with the above-mentioned thresholds, more than 50% of the genes in both patient cohorts had more over-expressed or under-expressed samples than the housekeeping genes.

### Boolean Implication Networks

The implication network algorithm used in this study was proposed by Guo et al. based on prediction logic. Formal logic rules relating two binary variables were inferenced for each pair of genes to form the implication network. In this study, the algorithm was improved by counting the error cell of each implication rule such that it can model not only the binary variable but also multivariate data. This network induction algorithm is generic for various general applications, including multinomial datasets and multi-classification problems.

The Boolean implication algorithm evaluates six implication rules: A→B (positive implication), A→¬B (forward negative implication), ¬A→B (inverse negative implication), ¬A→¬B (negative implication), A [symbol] B (positive equivalence), and A [symbol] ¬B (negative equivalence). (A &¬B) is the error cell for A→B, (A & B) is the error cell for A→¬B. (¬A &¬B) is the error cell for ¬A→B. (¬A & B) is the error cell for ¬A→¬B. (A &¬B) and (¬A & B) are the error cells for A [symbol] B. (A & B) and (¬A &¬B) are the error cells for A [symbol] ¬B.

The Boolean implication algorithm relies predominantly upon error cells for a particular rule while calculating the scope (Uₚ), indicating how much the data is covered, and precision (∇ₚ), indicating accuracy, for that rule. For instance, while looking for co-amplification in a pair of genes (A→B), the algorithm checks if a gene is amplified, and the remaining two states of deletion and normalcy become simply the negate of amplification; the error would be when gene A is amplified but gene B is not amplified. In this manner, the algorithm can use the same fundamental principle to create implication relations amongst genes, irrespective of the number of states for each gene.

For each implication rule to be significant, it must have a scope and precision value which is greater than the threshold value, and the greatest amongst all the rules for the pair of variables in question. The threshold for scope and precision depends on the significance level in statistical tests. The Algorithm 1 was described as:

```
  Begin
    Set a significance level ∇ₘᵢₙ and a minimal Uₘᵢₙ
    For nodeᵢ, i ∈ [0, νₘₐₓ − 1] and nodeⱼ, j ∈ [i +1, νₘₐₓ]
        (Note: νₘₐₓ is the total number of nodes)
      For all empirical case samples N, compute a contingency table:
```

| | |
|---|---|
| Mᵢⱼ = | N₁₁ N₁₂ |
| | N₂₁ N₂₂ |

For each relation type k out of the six cases find the solution:

| | |
|---|---|
| | Max Uₚ |
| Subject to | Max Uₚ > Uₘᵢₙ |
| | ∇ₚ > ∇ₘᵢₙ |
| | ∇_{error cells} > ∇_{non-error cells} |

If the solution exists, then return a type k relation
End

In the contingency table Mij, i stand for the index of the first gene (A) in the methodology, and j stand for the index of the second gene (B). One association type (i.e., co-amplification, etc.) was tested each time. N₁₁ represents the number of samples where both A and B have a true statement in the examined association type; N₁₂ is the count of samples where A is true, but B is not true; N₂₁ is the count of samples where A is not true, but B is true; N₂₂ is the number of samples where both A and B are not true. For a single error cell, where Nᵢⱼ is the number of error occurrences, N_{i⋅} is the row sum of contingency table Mᵢⱼ, and N_{⋅j} is the column sum of contingency table Mᵢⱼ. N is the total number, which is the sum of N₁₁, N₁₂, N₂₁, and N₂₂. The scope Uₚ and the precision ∇ₚ is defined as (2):

$${U}_{p} = {U}_{ij} = \frac{{N}_{i .} \times {N}_{. j}}{{N}^{2}} , {∇}_{p} = {∇}_{ij} = 1 - \frac{{N}_{ij}}{N \times {U}_{p}} \qquad ( 2 )$$

For multiple error cell types, the scope Uₚ and the precision ∇ₚ is defined as the Equations (3), where ωᵢⱼ₌1 for error cells; otherwise, ωᵢⱼ₌0.

$${U}_{p} = {\sum }_{i} {\sum }_{j} {ω}_{ij} \times {U}_{ij} , {∇}_{p} = {\sum }_{i} {\sum }_{j} ( \frac{{ω}_{ij} \times {U}_{ij}}{{U}_{p}} ) {∇}_{ij} \qquad ( 3 )$$

The pairwise gene relationships at the CNV level included co-amplification, co-deletion, amplification-deletion, and deletion-amplification. The pairwise gene relationships at gene expression level included co-upregulation, co-downregulation, upregulation-downregulation, and downregulation-upregulation. The modelled CNV and gene expression cross-level included amplification-upregulation, amplification-downregulation, deletion-upregulation, and deletion-downregulation. The scope and precision threshold can be adjusted by altering the statistical significance for the implication rules. The threshold value was calculated from a one-tailed z-test based on the sample size by setting the ideal z value. The z value used for this study was 1.64 (95% confidence interval, α=0.05, one-tailed z-tests).

### Cancer Cell Line Encyclopedia (CCLE)

Gene expression data for CCLE were downloaded from DepMap 20Q2 (https://figshare.com/articles/dataset/DepMap_20Q2_Public/12280541, accessed on 1 Apr. 2021). Gene expression data were obtained from the CCLE data portal (https://data.broadinstitute.org/ccle/CCLE_RNAseq_081117.rpkm.gct, accessed on 1 Apr. 2021). RNA-seq data were quantified using the GTEx pipelines. A total of 117 NSCLC cell lines were included in this analysis.

Proteomics data for CCLE were downloaded from the Gygi lab (https://gygi.hms.harvard.edu/publications/ccle.html, accessed on 1 Oct. 2021). A total of 63 NSCLC cell lines were included in this analysis.

### CRISPR-Cas9 Assays

Gene knockout effects in CCLE using CRISPR-Cas9 screens were quantified in Project Achilles. The data were obtained from DepMap 20Q2 (https://figshare.com/articles/dataset/DepMap_20Q2_Public/12280541, accessed on 1 Apr. 2021). The CRISPR-Cas9 data were processed with the CERES method. Gene effects in each cell line were normalized such that the median non-essential gene knockout effect is 0 and the median essential gene knockout effect is −1. A gene is defined as an essential gene if it is essential to the cell growth in each line; otherwise, it is defined as a non-essential gene. There were 78 NSCLC cell lines with genome-scale CRISPR-Cas9 knockout results. A normalized dependence score less than −0.5 indicates a significant effect in CRISPR-Cas9 knockout.

### RNAi Functional Assays

Genome-scale RNAi screening data in CCLE were obtained from Project Achilles (https://depmap.org/R2-D2/, accessed on 1 Apr. 2021). The DEMETER2 method was used to estimate average gene dependency scores in each cell line for short hairpin RNA (shRNA) libraries. Gene dependency scores were standardized with DEMETER2 such that the median of the across-cell-line average dependency scores of the positive control gene set was −1 and that of the negative control gene set was 0. There were 92 NSCLC cell lines with genome-scale RNAi screening results normalized with DEMTER2. A normalized dependence score less than −0.5 indicates a significant effect in RNAi knockdown.

### Immune Infiltration Estimation

TIMER 2.0 was used to find the association of gene expression and immune infiltration. TIMER is a comprehensive resource for systematically analyzing the immune infiltration of different cancer types (http://timer.cistrome.org/, accessed on 14 Jun. 2021). It provides the abundance of immune infiltration estimated by a variety of immune deconvolution methods.

### PRISM Drug Response in CCLE

The growth inhibitory activity of 4,518 drugs was quantified in 578 human cancer cell lines using the PRISM molecular barcoding and multiplexed screening method. The PRISM dataset is available at the Cancer Dependency Map portal (https://depmap.org/portal/download/, accessed on 1 Apr. 2021). Drug responses of 9 commonly used chemotherapeutic regimens in treating NSCLC were included in this study: carboplatin, cisplatin, paclitaxel, docetaxel, gemcitabine, vinorelbine, etoposide, gefitinib, and erlotinib. For each drug, cell lines with IC₅₀ or EC₅₀ value higher than the maximum dose were defined as resistant; cell lines with IC₅₀ or EC₅₀ value lower than the minimum dose were defined as sensitive. The remaining cell lines were divided into groups of resistant, sensitive, or partial response by using the mean±0.5 standard deviation (SD) of the drug activity metrics, including IC₅₀, ln(IC₅₀), EC₅₀, or ln(EC₅₀) values. Cell lines with a drug activity value less than the mean−0.5 SD were defined as sensitive to the drug, and those with a drug activity value between the mean+0.5 SD and the mean−0.5 SD were defined as having a partial response to the drug. Cell lines a drug activity value greater than the mean+0.5 SD were defined as resistant to the drug. This categorization corresponds to the RECIST 1.1 system (i.e., complete response, partial response, and stable disease/disease progression) in evaluating chemotherapeutic response in solid tumors.

### Genomics of Drug Sensitivity in Cancer (GDSC1/2)

Drug screening data were downloaded from Genomics of Drug Sensitivity in Cancer (GDSC) Project (https://www.cancerrxgene.org/downloads/bulk_download, accessed on 15 Apr. 2021). The GDSC Project screened more than 1,000 genetically characterized human cancer cell lines with a wide range of anti-cancer therapeutic agents. Among the commonly used chemotherapeutic regimens in treating NSCLC drugs, nine were found in GDSC1, including cisplatin, docetaxel, erlotinib, etoposide, gefitinib, gemcitabine, pemetrexed, and vinorelbine; seven were found in GDSC2, including cisplatin, docetaxel, erlotinib, gefitinib, gemcitabine, paclitaxel, and vinorelbine. For each drug, cell lines were defined as resistant, sensitive, or partial response by using the mean±0.5 SD of the IC₅₀ and ln(IC₅₀) values as described above.

### Drug Repurposing Using Connecitivity Map (CMap)

CMap (https://clue.io/, accessed on 1 Oct. 2021) was used to find perturbagen signatures that connected with gene expression signatures. It is a resource of gene expression profiles of pharmacologic perturbed cell lines, using transcriptional expression signatures to connect disease, cell physiology, and therapeutics.

### Western Blots

NSCLC cell lines H441, H23 and H1299 (kind gift of Dr. Scott Weed, WVU), A549 and H1975 (kind gift of Dr. Ivan Martinez, WVU) were grown in DMEM (Corning, cat. #15-018-CV) supplemented with 10% FBS (HyClone, UT, USA), 2 mM L-glutamine (Corning, NY, USA; cat. #25-005-CI), and 1×Antibiotic Antimycotic Solution (Corning, NY, USA; cat. #30-004-CI). All cells were maintained at 37° C. in a 5% CO2 incubator. Whole/total cell lysates were prepared in nonreducing Laemmli buffer as described in. Nuclear-cytoplasmic fractionation was performed using ProteoJET Cytoplasmic and Nuclear Protein Extraction Kit (Fermentas, cat. #K0311) as described in.

Protein concentration was quantified by Pierce BCA Protein Assay (ThermoFisher, MA, USA; cat #23225). Lysates with equal amount of total protein were separated on 4% to 12% Bis-Tris NuPAGE Novex gels (Invitrogen, MA, USA) and transferred to a polyvinylidene difluoride (PVDF) membrane. Protein bands were detected using standard chemiluminescence techniques using GE Healthcare Amersham Imager 680.

ZNF71 antibody was purchased from GeneTex (Cat. No. GTX116553). The following antibodies were included in Western blots: ZEB1 (Sigma, St. Louis, MO, USA; HPA027524), Vimentin (V-9; Santa Cruz Biotechnology, TX, USA; sc-6260), E-cadherin (BD Biosciences, NJ, USA; cat #610181), pan-Cytokeratin (C11, Santa Cruz Biotechnology, Dallas, TX, USA; sc-8018), PCNA (eBioSciences, 14-6748-81), and Tubulin α (Sigma, St. Louis, MO, USA; T9026). Standard chemiluminescence was used to detect the protein bands.

### Statistical Methods

Statistical analysis was performed using Rstudio version 1.4.1106. The multivariate Cox regression analysis was used to build the model of risk-scores. Survival analysis was performed using Kaplan-Meier analysis with the survival package in R. Log-rank tests were used to assess the difference in survival probability from different groups in Kaplan-Meier analyses. Differential gene mRNA and protein expression between two groups was evaluated with Student's t-tests, and a two-sided p-value<0.05 was considered statistically significant. Pearson's correlation test was used to find the relationship between two variables. One-tailed two proportion z-test was used to compare if two proportions are the same.

The present disclosure substantiates the feasibility of applying the 7-gene signature developed with qRT-PCR microfluidic assay to TCGA NGS data for NSCLC prognosis. CNV mediated transcriptional regulatory network of the 7-gene signature was identified in multiple NSCLC cohorts using Boolean implication networks. The multi-omics network genes, including PD-L1 (CD274), were significantly correlated with immune infiltration and drug sensitivity or resistance to 10 commonly used therapeutic regimens for treating NSCLC. ShRNA knock-down of NF-κB and cyclin D1 pathways were identified as associated with the multi-omics network for designing therapeutic strategies for NSCLC intervention. PI3K was identified as a potential pathway related to proliferation networks involving ZNF71 and its isoforms for future mechanistic study. Dasatinib, lestaurtinib, homosalate, FK 888, and penfluridol were discovered as potential repositioning drugs that can kill NSCLC cells, maintaining the expression of drug sensitivity genes and epithelial markers, and inhibit mesenchymal markers, proliferation genes, and ICIs for NSCLC treatment.

In NSCLC, there is a pressing need for immunotherapy predictive biomarkers. The processes underlying B-cell dysfunction, as well as their prognostic importance in NSCLC, are unknown. Tumor-specific B-cell gene co-expression networks were constructed by comparing Boolean implication modeling of single-cell RNA sequencing of NSCLC tumor B cells and normal B cells, respectively. Proliferation genes were selected from the networks using in vitro CRISPR-Cas9/RNA interfering (RNAi) screening data in more than 92 human NSCLC epithelial cell lines. The prognostic and predictive evaluation was performed using public NSCLC transcriptome and proteome profiles. A B-cell proliferation and prognostic gene co-expression network was present only in normal lung B cells but missing in NSCLC tumor B cells. A 9-gene signature was identified from this B-cell network that provided accurate prognostic stratification using bulk NSCLC tumor transcriptome (n=1,313) and proteome profiles (n=103). Multiple genes (HLA-DRA, HLA-DRB1, OAS1, and CD74) differentially expressed in NSCLC B cells, peripheral blood lymphocytes, and tumor T cells had concordant prognostic indications at mRNA and protein expression levels. The selected genes were associated with drug-sensitivity/-resistance to 10 commonly used NSCLC therapeutic regimens. Lestaurtinib was discovered as a potential repositioning drug for treating NSCLC.

Non-small cell lung cancer (NSCLC), accounting for 84% of lung cancer cases, is the leading cause of cancer-related mortality. The major histological subtypes that constitute NSCLC are lung adenocarcinoma (40% of NSCLC cases), squamous cell carcinoma (25-30%), and large cell carcinoma (5-10%), and each subtype represents a distinct prognosis for the patients as does the treatment option. Adjuvant chemotherapy of stage II/III NSCLC has resulted in 10-15% increased overall survival. However, the overall 5-year survival rate for NSCLC is less than 15% due to the limited therapeutic response and the resulted tumor recurrence/metastasis. Immunotherapy has shown promising results in NSCLC. Neoadjuvant PD-1 inhibitor nivolumab induced pathological response in 9 of 20 (45%) resected NSCLC tumors in stages I, II, and IIIA, including both PD-1 positive and negative tumors, with few side effects. In an open-label phase I clinical trial of chemotherapy-naive NSCLC patients with stage IIIB or stage IV, nivolumab with anti-CTLA4 ipilimumab in first-line therapy had an acceptable safety profile and demonstrated promising clinical performance, with a high response rate and long-term response. Patients with stage IIIB who received anti-PD-L1 durvalumab after chemoradiation had a significant overall survival benefit (median OS not reached in the durvalumab arm compared to 29.1 months in the placebo arm [HR 0.69 (95% CI 0.55-0.86)] in phase III randomized trial. The overall survival benefit of pembrolizumab mono-therapy was demonstrated in untreated stage IV NSCLC patients compared to chemo-therapy in patients with tumors expressing PD-L1 tumor proportion score (TPS) ≥ 50%, TPS ≥ 20% and TPS ≥ 1% in randomized phase III clinical trials. Atezolizumab received the FDA approval for stage II and IIIA NSCLC following chemotherapy in patients with PDL1>1%. Nevertheless, only a subset of NSCLC patients responded to immunotherapy due to primary, adaptive, or acquired immune resistance, mutations, and the varying amount and properties of tumor infiltrating lymphocytes (TILs). PDL1 and tumor mutational burdens haven't been demonstrated to be reliable predictive biomarkers. There are currently no well-established predictive biomarkers for immunotherapy response. In addition, there are no clinically applied biomarkers to identify early-stage NSCLC patients with all histological subtypes who are at high risk for tumor recurrence and metastasis for adjuvant therapies.

The tumor immune microenvironment is complex, dynamic, and heterogeneous, comprising interweaving signaling pathways and networks of genes and proteins within the immune system, stromal cells, and the host factors. When tertiary lymphoid structures (TLSs) are present in the tumor microenvironment, cancer patients typically have favorable clinical outcomes. TLSs in NSCLC tumors contain follicular B cells and adjacent clusters of dendritic cells and T cells. TLSs and tumor-infiltrating B cells increase immune checkpoint inhibitors (ICIs) responses in cancer immunotherapy, which has prognostic implications. Profiling of immune cell composition showed that only B cells had a significantly higher presence in tumors compared to the distal lung in an NSCLC patient cohort. A high density of B cells within TLS is positively correlated with tumor antigen-specific antibody responses, increased intratumor CD4+ T cell clonality, as well as early differentiated, activated, and non-regulatory CD4+ T cells, suggesting a central role of B cells in determining protective T cell responses in NSCLC patients. T cell dysfunction and therapy have been established for cancer treatment, including NSCLC. However, the biology, prognostic significance, and potential benefit of B cell-based immunotherapy in lung cancer have yet to be deciphered.

Given this intricacy and heterogeneity, a gene signature integrating various features should be created to identify the right patient for the specific immunotherapy, in concert with chemotherapy and/or radiotherapy. Novel computational network modeling can reveal essential molecular interactions with implications in prognosis, proliferation, and response to therapies for improving NSCLC treatment. Rigorous pathway and network approaches are crucial for the discovery of innovative targeted therapies and repurposing drugs to prolong NSCLC survival. This study utilized Boolean implication networks to identify tumor-specific B-cell gene co-expression networks in NSCLC using public single-cell RNA-sequencing data. Within the identified B-cell networks, proliferation genes were identified from CRISPR-Case9/RNA interference (RNAi) screening assays in human NSCLC cell lines. Differentially expressed genes in B cells and T cells with prognostic implications were selected using bulk NSCLC tumor transcrip-tome (n=1,313) and proteome profiles (n=103). Chemopredictive genes were filtered using public in vitro drug screening data in human NSCLC epithelial cell lines (n=135). Further, functional pathways, targeted therapies, and repositioning drugs were discovered based on the genes identified using the artificial intelligence (AI) network approach for improving NSCLC outcomes.

### Materials and Methods

### Public Patient Cohorts

Single-cell RNA Sequencing of B cells. Single-cell RNA sequencing data of 192 B-cell samples were generated from one NSCLC patient, including 96 tumor B-cell samples and 96 normal B-cell samples from adjacent normal lung tissues. Live CD45+ CD19+ cells were directly sorted in 2 μl of Qiagen TCL buffer in 96-well plates. The modified SmartSeq2 protocol was used to prepare the libraries. The RNA samples were analyzed with Illumina HiSeq 2000. Gene features were counted with the featureCounts method. based on the Gencode v19 transcriptome annotation. The data containing feature counts were available at the NCBI GEO with accession number GSE84789. Feature counts of 20,092 genes in all 192 samples were included to construct single-cell gene co-expression networks and perform differential expression analysis.

Single-cell RNA Sequencing of T cells from Peripheral Blood Lymphocytes (PBLs). Single-cell RNA sequencing data of T cells from PBLs was generated by Chiou et al. at Stanford FACS Facility. A total of 92 T-cell samples were collected from three NSCLC patients; 531 T-cell samples were collected from four healthy donors. The samples were sequenced using Illumina HiSeq 4000. The reads were mapped with STAR aligner. based on human genome reference GRCh38. SAMtools. were used to sort and index the mapped reads. The sorted PBL counts data was available at the NCBI GEO (GSE151531). PBL counts of 17,004 genes were used in differential expression analysis in this study.

Single-cell RNA Sequencing of NSCLC Tumor T cells. Tumor-infiltrating T-cell data was also provided in Chiou et al. A total of 2,950 T-cell samples were collected from 10 NSCLC patients. The data was sequenced, mapped, sorted, and indexed with the same process as for the PBL T-cell data described above. Chiou et. al. performed the dimension reduction using Uniform Manifold Approximation and Projection (UMAP) on this NSCLC tumor T-cell data and identified 14 cell clusters (C1-C14). The counts of 20,719 genes and the cell cluster identify were available at the NCBI GEO (GSE151537).

RNA Sequencing of bulk NSCLC Tumors. RNA sequencing data from 199 NSCLC patient tumor samples were collected. Sequencing was performed with Illumina HiSeq 2500. The reads were mapped using TopHat. based on human genome reference GRCh37. Raw read count was estimated using featureCounts. Fragments per kilobase million (FPKM) data was generated with Cufflinks. FPKM and read counts data were available at NCBI GEO (GSE81089). A total of 197 samples with sufficient survival information were included for prognostic evaluation.

Affymetrix microarray of NSCLC bulk Tumors. Affymetrix data of 100 NSCLC tumor samples was reported. This data included samples from 53 short-term (<20 months) survivors and 47 long-term (>58 months) survivors. Transcriptional expression profiles were generated with Affymetrix Human Genome U133 Plus 2.0 Array. Raw microarray data (GSE28571) was normalized using the robust multi-array average (RMA) method. This data was used to select prognostic genes in this study.

RNA Sequencing data of The Cancer Genome Atlas (TCGA). Two NSCLC cohorts from TCGA were obtained from LinkedOmics. (http://linkedomics.org/, accessed on 28 Apr. 2021). These cohorts included 515 lung adenocarcinoma (LUAD) samples and 501 lung squamous cell carcinoma (LUSC) samples with matched normalized RNA sequencing data and clinical follow-up information. The TCGA datasets were used for prognostic and radiotherapy response evaluation.

Proteomics of LUAD tumors. Proteomic quantification of 11,056 unique genes was measured in 103 LUAD tumors using LC-MS/MS. Log₁₀ transformed proteomic data was used in the prognostic evaluation in this study.

### Boolean Implication Networks

The Boolean implication network disclosed herein algorithm was used to construct single-cell gene co-expression networks in the present study. All implication rules representing gene co-expression relations are evaluated with scope and precision determined by the algorithm. Thresholds for scope and precision were computed by a one-tailed z-score test with the defined significance level and the sample size. z=3.891 (α=0.0001, one-tailed z-tests) was used for the calculation of thresholds. If an implication rule had both scope and precision value pass the threshold, that rule will be kept. Among all rules that were kept for the same pair of genes, only the one with the maximum precision was kept.

The normal and the NSCLC tumor-associated B-cell data were used to construct single-cell gene co-expression networks separately. In the data pre-processing for the Boolean implication network modeling, the sample with a feature count number greater than zero was denoted as “expressed”, and the sample with a feature count of zero was denoted as “not expressed” for the corresponding gene.

### Single-cell Differential Expression (DE) Analysis

The DE analysis of single-cell RNA sequencing data was performed with DEsingle. in Bioconductor. Genes with a p-value<0.05, an absolute log₂fold change (FC) value>1, and a false discovery rate (FDR)<0.25 were considered as significantly differentially expressed.

### Cancer Cell Line Encyclopedia (CCLE)

CCLE provides public access to comprehensive genetic characterizations for human cancer cell lines. Data of available NSCLC epithelial cell lines were used in this study. The datasets included RNA sequencing data quantified with the GTEx pipelines. (n=135, data obtained from https://portals.broadinstitute.org/ccle/data, accessed on 7 Dec. 2021) and proteomic data quantified with reverse-phase protein arrays. (n=63, data obtained from https://gygi.hms.harvard.edu/publications/ccle.html, accessed on 1 Oct. 2021).

### CRISPR-Cas9 Knockout Assays

Whole-genome CRISPR-Cas9 knockout screening results in CCLE cell lines were generated in Project Achilles. The genome-wide dependency scores in 94 NSCLC cell lines were obtained from the DepMap portal version 21Q4 data release. (https://depmap.org/portal/download/, accessed on 7 Dec. 2021). A normalized dependency score less than −0.5 indicates a significant effect of CRISPR-Cas9 knockout.

### RNAi Knockdown Assays

Whole-genome RNAi screening data for CCLE cell lines were also generated in Project Achilles. (https://depmap.org/R2-D2/, accessed on 1 Apr. 2021). The genome-wide dependency scores in 92 NSCLC cell lines were used for analysis in this study. RNAi knockdown was considered to have a significant effect if the normalized dependency score is smaller than −0.5.

### PRISM Drug Response in CCLE

The PRISM dataset. was obtained from the DepMap portal (https://depmap.org/portal/download/, PRISM repurposing 19Q4, accessed on 1 Apr. 2021). Carboplatin, cisplatin, docetaxel, erlotinib, etoposide, gefitinib, gemcitabine, paclitaxel (Taxol), pemetrexed, and vinorelbine were commonly used chemotherapeutic drugs in NSCLC treatment, the analysis focused on these ten regiments. Drug activity measurements, including IC₅₀, ln(IC₅₀), EC₅₀, and ln(EC₅₀), were used in the analysis. The available CCLE NSCLC cell lines were divided into three groups by the drug activity measurements: sensitive, resistant, and intermediate.

### Genomics of Drug Sensitivity in Cancer (GDSC)

Drug activity measurements were generated in the GDSC Project. (https://www.cancerrxgene.org/downloads/bulk_download, accessed on 15 Apr. 2021). IC₅₀ and ln(IC₅₀) were the main metrics used in this analysis. The available CCLE NSCLC cell lines were divided into three groups by the drug activity measurements in the GDSC1/2 datasets, i.e., sensitive, resistant, and intermediate.

### Drug Repurposing Using Connectivity Map (CMap)

CMap provides a tool to identify genes, drugs, and disease states connected with gene expression signatures. By matching input gene expression signatures, candidate functional pathways and repositioning drugs can be generated with CMap. The results with a p-value<0.05 and a connectivity score>0.9 can be used as hypotheses for further investigation. The up-regulated and down-regulated gene lists discovered in this study were used as database queries to find the connected functional pathways and reposition drugs.

### Pathway Enrichment Analysis Using ToppGene

ToppGene. Suite (https://toppgene.cchmc.org) provides several tools for interpreting gene functional classification and enrichment analysis. The ToppFun tool of the ToppGene suite was used to find the statistical enrichment of pathways in this study.

### Statistical Methods

RStudio (version 1.4.1106), an integrated development environment for R, was used as the main tool for performing the statistical analyses. A two-tailed, unpaired Student's t-test was used to evaluate the significance of differential expression between two groups. Survival analysis and curves were generated using the Kaplan-Meier method, and log-rank tests were used to assess the difference in survival probability between different groups. The univariate and multivariate Cox regression analyses were used in the prognostic evaluation and curve generation with R packages “survival”, “survminer”, and “ggplot2”. The strength of the linear relationship between two sample groups was measured with Pearson's correlation test. Results in statistical tests were considered significant with a p-value<0.05.

### Results

Tumor-Specific Gene Co-Expression Networks in NSCLC B cells

Using the Boolean implication network algorithm, whole-genome mRNA co-expression networks were generated with single-cell RNA sequencing data of normal and NSCLC tumor B cells, respectively. The constructed tumor and normal B-cell gene co-expression networks were compared to identify tumor-specific B-cell co-expression networks, i.e., gene co-expression relations that existed only in tumor B cells but not in normal B cells, and vice versa. To obtain a manageable amount of network edges, the significant (p<0.00005, one-tailed z-tests) gene associations (i.e., implication rules) were selected for further analysis. A total of 232 significant gene co-expression relations (network edges) existed only in the NSCLC tumor B-cell network but not in the normal B-cell network, and 615,332 significant co-expression relations existed only in the normal B-cell network but not in the NSCLC tumor B-cell network. These identified tumor-specific B-cell gene co-expression networks were included in further analysis.

### DE Genes in NSCLC B Cells and T Cells With Prognostic Implications

FIGS. 14A-F illustrate examples of identified proliferation and prognostic gene co-expression network in NSCLC B cells, in accordance with various embodiments of the present disclosure.

In FIG. 14A, the shown gene co-expression network was only present in normal B cells and missing in NSCLC tumor B-cells. None of the co-expression relations was present in tumor B cells. All genes were significantly differentially expressed in NSCLC tumor-associated B cells vs. B cells in adjacent normal lung tissues. The intermediate genes were in the ellipse circles. These genes were not in the selected proliferation or prognostic gene list, but the selected genes were connected through these intermediate genes. The solid line indicates the direct connections between the selected genes, and the dashed line indicates the connections through the intermediate genes.

The significant DE genes (p<0.05, |log₂FC|>1, and FDR<0.25) were included in tumor vs. normal B cells in the next analysis. A total of 1,086 unique genes were selected, including 1,082 genes from the network only present in the normal B cells and 35 genes from the network only present in NSCLC tumor B cells.

Proliferation and prognostic genes were further selected from these 1,086 DE genes. The proliferation genes were selected that had a significant effect in both CRISPR-Cas9 and RNAi screening assays in more than 50% of the tested human NSCLC epithelial cell lines (FIG. 14A). The prognostic genes were selected that had a significant hazard ratio (HR, p<0.05, univariate Cox model) in the survival analysis of an NSCLC cohort (GSE81089), and significant differential expression (p<0.05, two-sample t-tests) between short-and long-term NSCLC survivors (GSE28571). The selected prognostic genes had a concordant association with patient survival in both cohorts.

The identified proliferation and prognostic gene co-expression network (FIG. 14A) was only present in the normal B cells but missing in the NSCLC tumor B cells. Some genes were not directly connected with the network but connected through one of the intermediate genes differentially expressed in NSCLC tumor B cells. ADCY2 and FGF5 were not connected with the network directly or through any intermediate genes. The B-cell proliferation and prognostic network pathway analyses were conducted with ToppGene.

FIG. 14B shows −log10(p-value) of the top 10 significantly enriched pathways in the ToppGene functional enrichment analysis of the proliferation and prognostic network. FIG. 14C shows a principal component analysis (PCA) using all the genes shown in FIG. 14A, which generated a clear separation of normal and NSCLC tumor B cells in single-cell RNA sequencing data.

From the B-cell proliferation and prognostic gene co-expression network (FIG. 14A), a 9-gene prognostic signature was identified in the multivariate Cox model from the training set (GSE81089) and was validated in the combined TCGA-LUAD and TCGA-LUSC data of NSCLC patients with stage I, II, or IIIA. In the multivariate Cox model analysis, a stepwise gene selection method was used to construct the model in the training set. In each iteration, the least statistically significant gene variable in the multivariate Cox model was dropped. This iteration was repeated until the model with the optimal prognostication in the training set was achieved. This model was then validated on the test set (TCGA data) with all the training parameters fixed. The nine marker genes were selected in this process. These nine genes included ANAPC5, CCT3, EWSR1, EXOC4, HRH1, MAP4, PPP2R1A, TUBA1B, and VCP.

FIG. 14D shows a Kaplan-Meier analysis of the 9-gene signature using RNA-sequencing data in the training set GSE81089. FIG. 14E shows a Kaplan-Meier analysis of the 9-gene signature using RNA-sequencing data in the TCGA-LUAD & TCGA-LUSC validation set. The Kaplan-Meier analysis results showed that the patients with a risk-score lower than 0.525 survived significantly longer than the patients with a risk-score higher than 0.525 in both training (p=0.00056, HR: 3.345 [1.616, 6.925]; FIG. 14D) and validation cohorts (p=0.0010, HR: 1.435 [1.155, 1.783]; FIG. 14E).

FIG. 14F shows a Kaplan-Meier analysis of the 9-gene signature using protoeomic data of MAP4 and VCP in LUAD patients. The protein expression of the 9-gene marker panel also provided significant patient stratification in a LUAD patient cohort. Only MAP4 and VCP were included in the proteomic multivariate Cox model due to data availability and the univariate significance. The Kaplan-Meier analysis results showed that the patient group with a risk-score lower than −6.56 survived significantly longer than the patient group with a risk-score higher than −6.56 (p=0.0048, HR: 3.807 [1.403, 10.33]; FIG. 14F).

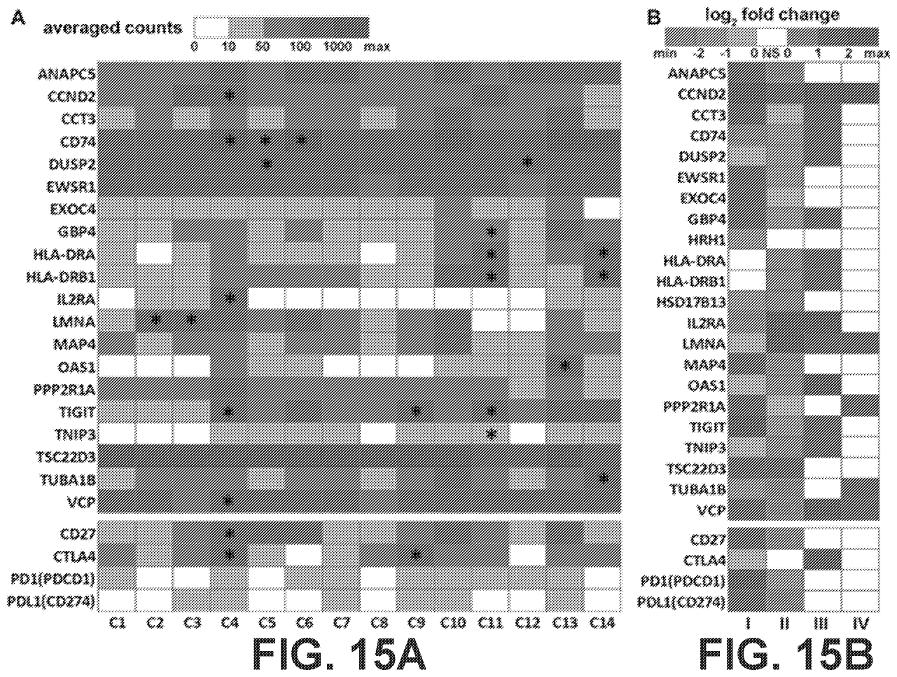

Interactions and coordination between B cells and T cells are crucial in antibody generation and immune protection. To gain a better understanding of genes relevant in NSCLC immunogenetics, DE genes in T cells in NSCLC PBL and tumors were examined next. Firstly, significant DE genes (p<0.05, |log₂FC|>1, and FDR<0.25) between normal and NSCLC PBL T cells (GSE151531) were identified. Secondly, this list of DE genes was compared with two T-cell DE gene lists from published studies. One published gene list. contained DE genes (p<0.05, two-sided moderated t-tests) between suppressive tumor Tregs of CD4-C9-CTLA4 cells (n=868) vs. other tumor-infiltrating Tregs of CD4-C8-FOXP3 cells (n=122), and DE genes (p<0.05, two-sided moderated t-tests) between activated tumor Tregs of CD4-C9-CTLA4 (TNFRSF9⁺, n=519) vs. non-activated tumor Tregs of CD4-C9-CTLA4 (TNFRSF9⁻, n=420). The other published gene list. consisted of the DE genes in each of the 14 T-cell clusters. Eleven genes, including CCND2, CD74, DUSP2, GBP4, HLA-DRA, HLA-DRB1, IL2RA, LMNA, OAS1, TIGIT, and TNIP3, were the common DE genes in all three lists. Two genes, HSD17B13 and TSC22D3, had concordant significant DE (p<0.05, |log₂FC|>1, and FDR<0.25) between NSCLC vs. normal PBL T cells (GSE151531) and B cells in the lung tissues (GSE84789). These selected 22 genes, including 13 DE genes and the 9-gene prognostic marker panel identified from the B-cell network (FIG. 14A), and 4 ICIs (CD27, CTLA4, PD1, and PDL1) were visualized in heatmaps (FIG. 15).

FIGS. 15A-B illustrate examples of differential expression patterns of selected genes in NSCLC T cells and B cells, in accordance with various embodiments of the present disclosure. FIG. 15A shows a heatmap of the average expression of the selected genes in 14 NSCLC tumor T cell clusters. The asterisk (*) indicates that the gene was significantly differentially expressed in the corresponding T cell cluster. FIG. 15B shows a heatmap of the log2(FC) patterns of the selected genes in this study. I. NSCLC tumor B cells (n=96) vs. normal B cells (n=96). II. Peripheral blood lymphocyte T cells from NSCLC patients (n=531) vs. healthy donors (n=92). III. NSCLC suppressive tumor Tregs of CD4-C9-CTLA4 cells (n=868) vs. other tumor-infiltrating Tregs of CD4-C8-FOXP3 cells (n=122). IV. NSCLC Activated tumor Tregs of CD4-C9-CTLA4 (TNFRSF9+, n=519) vs. non-activated tumor Tregs of CD4-C9-CTLA4 (TNFRSF9-, n=420).

CTLA4, PD1, and PDL1 are well-established immunotherapy targets in NSCLC. CD27 is a new ICI. that is being investigated in phase I/II clinical studies for a variety of cancer types, with encouraging results. CD27 was also included in a 7-gene prognostic and chemopredictive assay, showing concordant prognostic indications at mRNA and protein expression levels in NSCLC tumors. Here, the expression of these ICIs in NSCLC tumor B cells, T cells, and PBL T cells in single-cell RNA sequencing profiles was examined. The average expression of these genes in the 14 NSCLC T-cell clusters. was shown in FIG. 15A and their DE patterns in NSCLC T cells and B cells were shown in FIG. 15B. HRH1 and HSD17B13 were not available in the NSCLC tumor T cell dataset. and therefore were not included in FIG. 15A.

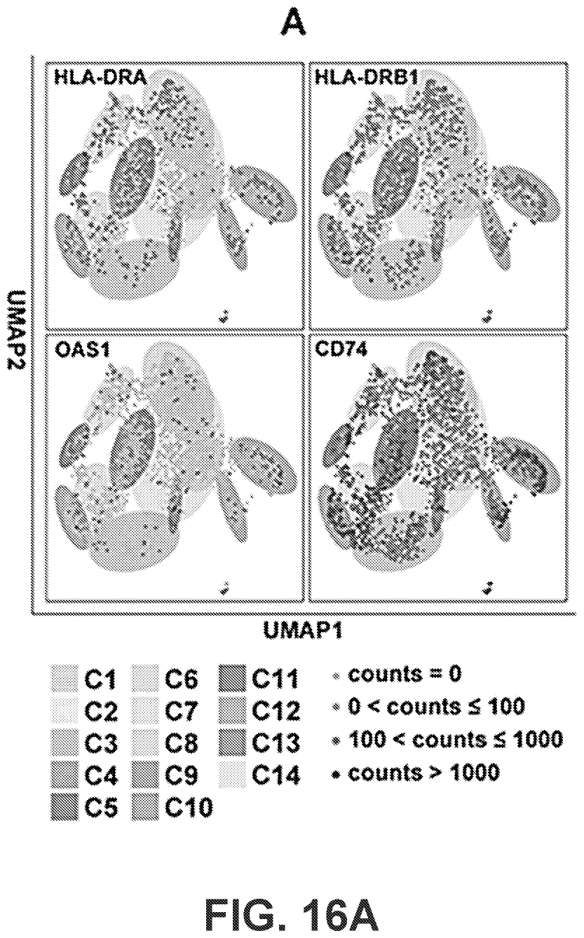

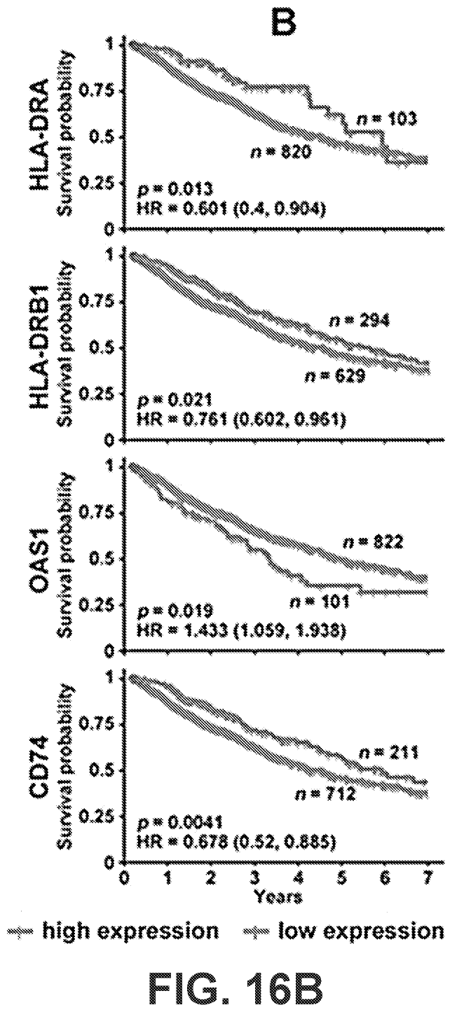

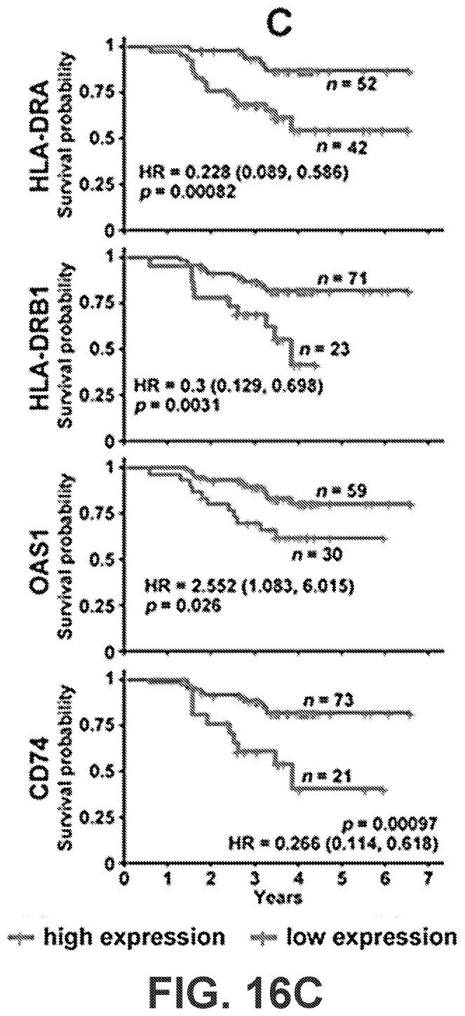

FIGS. 16A-B illustrate examples of differentially expressed genes in NSCLC T cells with prognostic indications, in accordance with various embodiments of the present disclosure. FIG. 16A shows an expression of HLA-DRA, HLA-DRB1, OAS1, and CD74 in 2,950 single T cells across 14 clusters illustrated in the UMAP layout. FIG. 16B shows a Kaplan-Meier analysis of TCGA-LUAD & TCGA-LUSC patients stratified based on mRNA expression of HLA-DRA, HLA-DRB1, OAS1, and CD74 in the RNA-sequencing data, respectively. (C) Kaplan-Meier analysis of patients in the Xu cohort stratified based on the protein expression of HLA-DRA, HLA-DRB1, OAS1, and CD74, respectively.

Fourteen major distinct T cell states of activation/exhaustion were identified, of which 13 (C1 to C13) could be linked to the cell states reported by Guo et al. using a different patient cohort. Cluster C14 represented CD4⁺ and CD8⁺ T cells in the cell cycle. Clusters C5, C6, and C12 were CD8⁺ T cells with effector phenotypes. Clusters C7 and C10 were CD8⁺ T cells with a resident memory phenotype. C11./CD8-C6-LAYN. (CD8⁺ exhausted T cells) and C4./CD4-C9-CTLA4. (CD4⁺ Tregs) were almost entirely consisted of cells originated from tumors. Clusters C5, C6, C7, and C12 were inferred to exhibit virus-specific T cell states.

Among the selected genes (FIG. 15), HLA-DRA, HLA-DRB1, OAS1, and CD74 had concordant prognostic indications at mRNA and protein expression levels in bulk NSCLC tumors with stage I, II, or IIIA (FIG. 15). FIG. 15A presented their mRNA expression in 2,950 single NSCLC T cells across 14 clusters (GSE151537) illustrated in the UMAP layout. HLA-DRA and HLA-DRB1 were both DE genes in C11./CD8-C6-LAYN. (CD8⁺ exhausted T cells) and the C14 cluster of CD4⁺ and CD8⁺ cells in the cell cycle. (FIG. 14A). OAS1 was a DE gene in C13./C4-GZMK and C4-CD69. CD4⁺ cells. CD74 was a DE gene in C4./C9-CTLA4. CD4⁺ cells and the C5 and C6 clusters of CD8⁺ effector T cells. The results showed that the patients with a higher expression of HLA-DRA, HLA-DRB1, and CD74 survived significantly longer (p<0.05, Kaplan-Meier analysis) than those with a lower expression of these genes at both mRNA (FIG. 15B) and protein levels. (FIG. 15C). When OAS1 expressed higher at both mRNA and protein levels, patients survived significantly shorter (p<0.05, Kaplan-Meier analysis) in both TCGA-LUAD & TCGA-LUSC patients (FIG. 15B) and the proteomic LUAD cohort from Xu et al. (FIG. 15C). Although different patient cohorts were used in the single-cell and prognostic analysis, these results indicate the potential utility of the identified genes in single B-cell and T-cell analysis of NSCLC patient tumors and PBL for diagnosis and prognosis.

### Genes Associated With Response to Radiotherapy and Chemotherapy

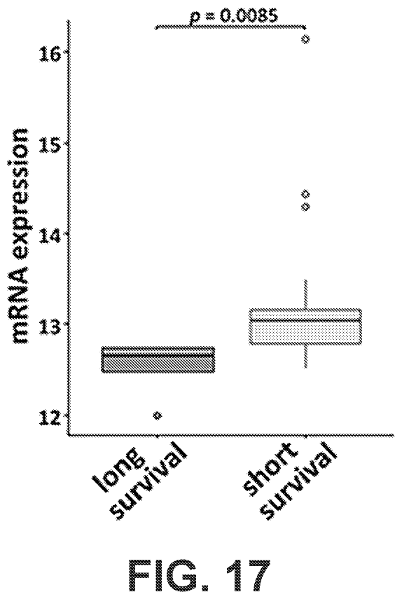

FIG. 17 illustrates an example of the mRNA expression of VCP was associated with resistance to radiotherapy. The studied patient cohort was TCGA-LUSC and TCGA-LUAD stage III and stage IV patients who had been treated with radiotherapy. VCP showed significantly higher expression level (p=0.0085, two-sample student t-tests) in the short-survival patient group (<20 months; n=186) compared with the long-survival patient group (>58 months; n=144).

To assess the association with radiotherapy response, the mRNA expression of the selected genes (FIG. 15) was evaluated in TCGA-LUAD and TCGA-LUSC cohorts. The cohorts only included the stage III and stage IV patients who received radiotherapy (n=56). VCP had a significantly higher mRNA expression level (p<0.05, two-sample Student's t-tests) in the short-survival (<20 months) patient group than in the long-survival (>58 months) patient group (FIG. 17). The averaged log₂ transformed RPKM of VCP was 12.52 in the long-survival patient group and 13.22 in the short-survival patient group. The average original RPKM of VCP was 5975.11in the long-survival patient group and 12367.55 in the short-survival patient group. The fold change of VCP was 2.07 in the short-survival vs. the long-survival patient group who received radiotherapy using the original gene expression measurements.

The association of the 22 selected DE genes (FIG. 15) with drug sensitivity profiles of selected NSCLC chemotherapeutic drugs in 135 NSCLC cell lines was next examined. The lists of genes that significantly differentially expressed (p<0.05; two-sample t-tests) in sensitive NSCLC cell lines vs. resistant NSCLC cell lines at mRNA level (Table 8) and protein level (Table 9) were provided.

TABLE 8
Genes with a significant differential expression (p < 0.05; two-sample t-tests) in
mRNA expression in sensitive vs. resistant NSCLC cell lines (n = 135) to the selected
regiments. The genes in BOLD font were drug-sensitive genes which expressed higher in
sensitive cell lines; the genes in REGULAR font were drug-resistant genes that expressed
higher in resistant cell lines.

| | GDSC1 | GDSC2 | PRISM |
|---|---|---|---|
| Carboplatin | | | DUSP2, HLA-DRB1, TIGIT, TNIP3 |
| Cisplatin | | DUSP2 | ANAPC5, OAS1, VCP, IL2RA |
| Docetaxel | LMNA, PPP2R1A, | LMNA | IL2RA, CCND2, PPP2R1A |
| | TNIP3 | | |
| Erlotinib | DUSP2, TSC22D3 | HRH1, LMNA | HRH1, LMNA |
| Etoposide | CCT3, EWSR1, | | CCT3, VCP, HSD17B13 |
| | TUBA1B | | |
| Gefitinib | VCP, HSD17B13 | LMNA, MAP4, TUBA1B | CCND2, CCT3, HRH1, |
| | | | HSD17B13 |
| Gemcitabine | | CCT3 | CD74, DUSP2, HLA-DRA, TIGIT |
| Paclitaxel | | DUSP2, VCP | OAS1, PPP2R1A, TNIP3, |
| | | | HSD17B13 |
| Pemetrexed | CCT3, DUSP2 | | |
| Vinorelbine | EXOC4 | CCT3, DUSP2, LMNA, | CCND2, DUSP2, HRH1, LMNA |
| | | VCP | |

TABLE 9
Genes with a significant differential expression (p < 0.05; two-sample t-tests) in
protein expression in sensitive vs. resistant NSCLC cell lines (n = 63) to the selected
regiments. The genes in BOLD font were drug-sensitive genes that expressed higher in
sensitive cell lines; the genes in REGULAR font were drug-resistant genes that expressed
higher in resistant cell lines.

| | GDSC1 | GDSC2 | PRISM |
|---|---|---|---|
| Carboplatin | | | |
| Cisplatin | LMNA | | CCT3, OAS1, VCP, PPP2R1A |
| Docetaxel | PPP2R1A, TUBA1B | | GBP4 |
| Erlotinib | | CCT3 | VCP |
| Etoposide | | | LMNA |
| Gefitinib | CCT3, HLA-DRA, TSC22D3 | CCT3 | CCT3, OAS1 |
| Gemcitabine | | EXOC4 | CD74, LMNA, VCP |
| Paclitaxel | | | CD74, HLA-DRA, TUBA1B |
| Pemetrexed | ANAPC5, HRH1 | ANAPC5, HRH1 | |
| Vinorelbine | | | |

### Discovery of Targeted Therapies and Repositioning Drugs

A 7-gene prognostic and predictive assay for early-stage NSCLC (including ABCC4, CCL19, SLC39A8, CD27, FUT7, DAG1, and ZNF71) has been developed. ZNF71 protein and its KRAB isoform gene expression are associated with epithelial to mesenchymal transition (EMT). A 14-gene classifier characterizing NSCLC tumor EMT states can be associated with distinct patient survival outcomes. To improve NSCLC treatment outcomes, targeted therapies and repositioning drugs were searched in the CMap database to prolong survival, enhance drug response, inhibit NSCLC proliferation, and reverse EMT. The CMap input gene list included the 9-gene prognostic marker panel identified from the B-cell network (FIG. 14A), drug-sensitive and-resistant genes (Table 7), 6 proliferation genes with a significant effect in both CRISPR-Cas9 and RNAi assays of all the tested human NSCLC cell lines, and EMT genes used in a previous CMap analysis. Drug-sensitive/resistant genes with conflict results between mRNA (Table 8) and protein expression (Table 9) were removed. One additional gene, CDK7, was added to the CMap input gene list. CDK7 was associated with DAG1 in a 7-gene NSCLC prognostic and predictive marker panel. in the NSCLC B-cell networks. Specifically, a co-upregulation of CDK7 and DAG1 was observed in normal lung B cells, but not in the tumor B cells (p<0.05, one-tailed z-tests). CDK7 was significantly overexpressed (log₂FC=2.92, p<0.05, and FDR<0.25) in NSCLC tumor B cells vs. normal B cells. CDK7 was a survival-hazard gene with an HR greater than 1 (p<0.05, univariate Cox model) in GSE81089 and a fold change (short-survival/long-survival) greater than 1 (p<0.05, two-sample t-tests) in GSE28571. CDK7 had a significant knockout effect in CRISPR-Cas9 assays in all 94 tested human NSCLC cell lines. The mRNA expression of CDK7 was associated with drug resistance to cisplatin and erlotinib in human NSCLC cell lines (n=135).

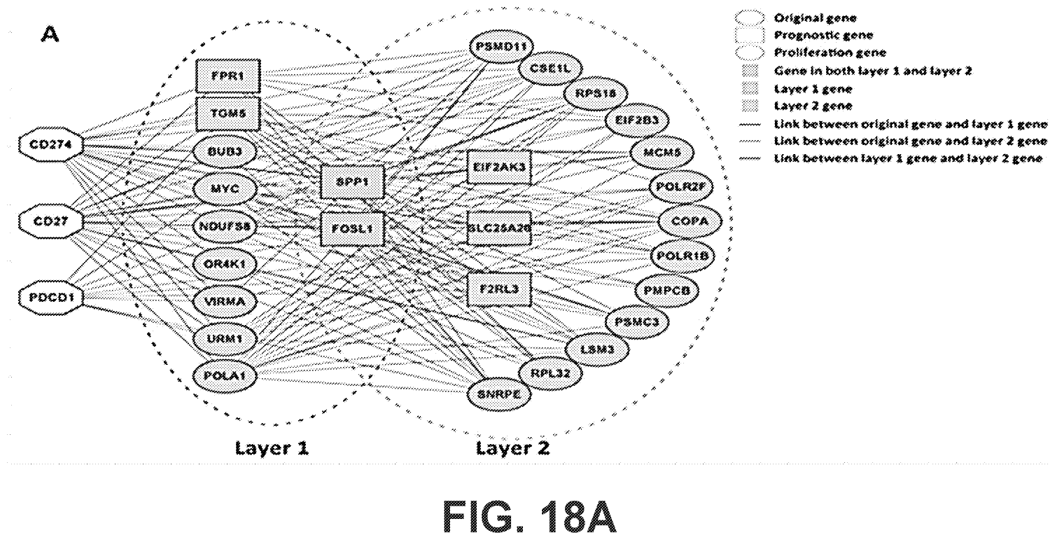

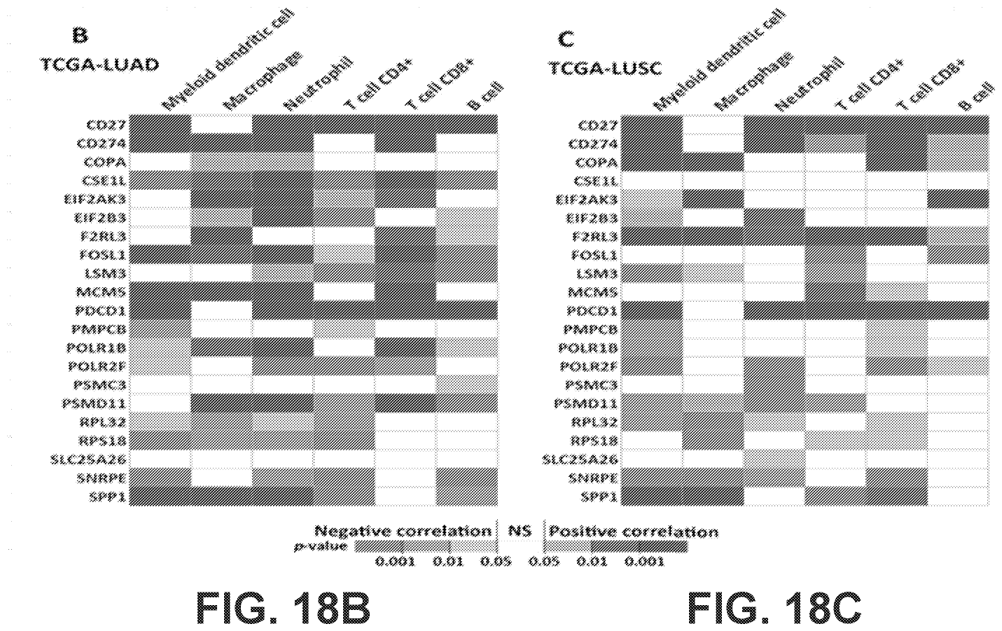

The following mechanisms were applied to identify functional pathways, targeted therapies, and repositioning drugs for improving NSCLC treatment: (1) up-regulation of survival-protective genes; (2) down-regulation of survival-hazard genes; (3) up-regulation of drug-sensitive genes; (4) down-regulation of drug-resistant genes; (5) down-regulation of proliferation genes; (6) up-regulation of the epithelial markers; (7) down-regulation of mesenchymal markers. The input up-regulated and down-regulated gene lists (FIG. 18A) for CMap analysis were created based on these mechanisms, and genes present in both up-, and down-regulated lists were removed.

FIGS. 18A-C illustrate examples of discovery of repositioning drugs and functional pathways based on selected genes, in accordance with various embodiments of the present disclosure. FIG. 18A shows a selection of significant functional pathways and repositioning of drugs based on the identified B-cell proliferation and prognostic network with CMap. FIG. 18B shows the Pearson correlation of CD27 mRNA expression and danusertib EC50 in NSCLC cell lines (n=79). FIG. 18C shows CMap selected compounds that had a low average concentration of drug response (IC50 and EC50) in the CCLE NSCLC cell lines (nlestaurtinib=67, nTW-37=81, ndanusertib=80).

Several targeted therapeutic candidates were identified with CMap. First, compounds were selected as targeted therapies that had a significant negative correlation (p<0.05, Pearson's correlation test) between drug-response measurements [IC₅₀, ln(IC₅₀), EC₅₀, ln(EC₅₀)] and gene expression of one of four ICIs (CD27, PD1, CTLA4, or PDL1) in human NSCLC epithelial cell lines (n=135). EC₅₀of danusertib had a significant negative correlation (p<0.05, Pearson's correlation test) with CD27 mRNA expression (FIG. 18B). Second, drugs were selected if they had a low average IC₅₀ and EC₅₀ in human NSCLC epithelial cell lines (n=135), indicating their efficacy in inhibiting NSCLC cell growth. Three small molecules were selected, including danusertib, lestaurtinib, and TW-37 (FIG. 18C), which had small average IC₅₀ and EC₅₀ values (less than 1 μM) without any outliers (IC₅₀ or EC₅₀ higher than 10 μM) in NSCLC cells.

Lestaurtinib is a tyrosine kinase inhibitor of fms-like tyrosine kinase 3 (FLT3) for treating AML. The results from this study suggest that lestaurtinib could be repositioned to improve NSCLC treatment when combined with existing therapies. Evidence supports the use of the other selected small molecules for NSCLC treatment. Danusertib, an aurora kinase inhibitor, was investigated in treating advanced solid tumors including NSCLC in phase I. and phase II. clinical studies, in which single-agent danusertib was well tolerated and showed marginal anti-tumor activity in common solid tumor types. TW-37, a small molecule inhibitor of B-cell lymphoma 2 (Bcl-2) family proteins, enhanced the pro-apoptosis and anti-migration ability of gefitinib in NSCLC. TG-101348, an inhibitor of Janus kinase 2 (JAK2), reduced PDL1 protein expression. and reversed erlotinib-resistance in NSCLC cells. TG-101348 mediated radiosensitization and EMT blockade in a xenograft mouse model of lung cancer. These results in the literature support the effectiveness of the presented artificial intelligence pipeline for identifying targeted therapies and repositioning drugs.

Significant functional pathways (p<0.05, connectivity score>0.9) in overexpression and shRNA knockdown assays of NSCLC cell lines were identified using CMap that matched the input gene expression signature (Table 10). The identified significant functional pathways included metabolism, VEGF, DNA synthesis and repair, protein regulation, ribosomal subunit (40S) relevant in the scanning of mRNAs and initiation of protein synthesis, and hallmarks including PI3K, STAT3, and BRAF, etc., in shRNA knockdown assays. Overexpression of cell cycle inhibition was also identified as a significant functional pathway (Table 10). It should be noted that although the results from various functional assays were used to select these significant pathways via bioinformatics matching, they can only be interpreted as hypotheses for future investigations.

TABLE 10
The significant (p < 0.05, connectivity score > 0.9) consensus signatures from overexpression and shRNA knockdown
targeted the same genes in NSCLC cell lines in CMap. OE: overexpression assay. KD: knockdown. SH: shRNA assay.

| | cell_ | | |
|---|---|---|---|
| src_set_id | name | pert_type | genes |
| OE_CELL CYCLE_INHIBITION | A549 | TRT_OE | CDKN1A, CDKN1B, CDKN2C |
| BIOCARTA_AHSP_PATHWAY | A549 | TRT_SH.CGS | AHSP, ALAD, ALAS1, ALAS2, CPOX, FECH, GATA1, HBA1, HBA2, |
| | | | HBB, HMBS, UROD, UROS |
| KD_AHSP_PATHWAY | A549 | TRT_SH.CGS | ALAD, FECH, GATA1, UROD |
| KD_RIBOSOMAL_40S_SUBUNIT | A549 | TRT_SH.CGS | FAU, RPS3, RPS3A, RPS5, RPS6, RPS7, RPS9, RPS10, RPS13, |
| | | | RPS14, RPS15A, RPS16, RPS19, RPS27A |
| KEGG_TAURINE_AND_HYPOTAURINE_ | A549 | TRT_SH.CGS | ADO, BAAT, CDO1, CSAD, GAD1, GAD2, GGT1, GGT5, GGT6, |
| METABOLISM | | | GGT7 |
| KEGG_TERPENOID_BACKBONE_ | A549 | TRT_SH.CGS | ACAT1, ACAT2, DHDDS, FDPS, GGPS1, HMGCR, HMGCS1, |
| BIOSYNTHESIS | | | HMGCS2, IDI1, IDI2, MVD, MVK, PDSS1, PDSS2, PMVK |
| OE_PHOSPHOLIPASES | A549 | TRT_SH.CGS | PLCG2, PLA2G12B, PLCB1, PLD1 |
| PID_VEGF_VEGFR_PATHWAY | HCC515 | TRT_SH.CGS | FLT1, FLT4, KDR, NRP1, NRP2, PGF, VEGFA, VEGFB, VEGFC, |
| | | | VEGFD |
| REACTOME_HOMOLOGOUS_RECOMB | A549 | TRT_SH.CGS | ATM, BRCA1, BRCA2, BRIP1, H2AX, LIG1, MDC1, MRE11, NBN, |
| INATION_REPAIR_OF_REPLICATION_ | | | RAD50, RAD51, RAD52, RPA1, RPA2, RPA3, TP53BP1 |
| INDEPENDENT_DOUBLE_STRAND_ | | | |
| BREAKS | | | |
| REACTOME_HYALURONAN_ | A549 | TRT_SH.CGS | ABCC5, CD44, CEMIP, CHP1, GUSB, HAS1, HAS2, HAS3, HEXA, |
| METABOLISM | | | HEXB, HMMR, HYAL1, HYAL2, HYAL3, LYVE1, SLC9A1, STAB2 |
| REACTOME_HYALURONAN_UPTAKE | A549 | TRT_SH.CGS | CD44, CHP1, GUSB, HEXA, HEXB, HMMR, HYAL1, HYAL2, HYAL3, |
| AND_DEGRADATION | | | LYVE1, SLC9A1, STAB2 |
| REACTOME_PROCESSIVE_SYNTHESIS_ | A549 | TRT_SH.CGS | DNA2, FEN1, LIG1, PCNA, POLA1, POLA2, POLD1, POLD2, POLD3, |
| ON_THE_LAGGING_STRAND | | | POLD4, PRIM1, PRIM2, RPA1, RPA2, RPA3 |
| REACTOME_REMOVAL_OF_THE_FLAP | A549 | TRT_SH.CGS | DNA2, FEN1, PCNA, POLD1, POLD2, POLD3, POLD4, RPA1, RPA2, |
| _INTERMEDIATE_FROM_THE_C_ | | | RPA3, WRN |
| STRAND | | | |
| REACTOME_REPAIR_SYNTHESIS_ | A549 | TRT_SH.CGS | PCNA, POLD1, POLD2, POLD3, POLD4, POLE, POLE2, RFC2, |
| FOR_GAP_FILLING_BY_DNA_POL_IN_ | | | RFC3, RFC4, RFC5, RPA1, RPA2, RPA3 |
| TC_NER | | | |
| REACTOME_SIGNAL_REGULATORY_ | A549 | TRT_SH.CGS | CD47, FYB, GRB2, PTK2, PTK2B, PTPN6, SIRPA, SIRPB1, SIRPG, |
| PROTEIN_SIRP_FAMILY_ | | | SRC, TYROBP |
| INTERACTIONS | | | |
| ST_G_ALPHA_I_PATHWAY | A549 | TRT_SH.CGS | AKT1, AKT2, AKT3, ASAH1, BRAF, CFB, DAG1, DRD2, EGFR, |
| | | | EPHB2, GRB2, ITPKA, ITPKB, ITPR1, ITPR2, ITPR3, KCNJ3, KCNJ5, |
| | | | KCNJ9, MAPK1, PI3, PIK3CB, PITX2, PLCB1, PLCB2, PLCB3, |
| | | | PLCB4, RAF1, RAP1GAP, RGS20, SHC1, SOS1, SOS2, SRC, |
| | | | STAT3, TERF2IP |

### Discussion

The present disclosure identified a gene co-expression network observed only in normal B cells (FIG. 14A) and missing in NSCLC tumor B cells. The transcriptomic and proteomic profiles of the 9-gene prognosis marker panel identified from this B-cell network accurately stratified resectable NSCLC patients into different groups with distinct survival outcomes. A similar approach was applied to dissect tumor-specific B-cell proliferation networks involving PD1 (PDCD1), PDL1 (CD274), and CTLA4. Each gene associated with these three established ICIs was significantly differentially expressed in NSCLC tumor B cells vs. normal B cells and had a significant impact on proliferation in CRISPR-Cas9 and/or RNAi assays in more than 50% of the tested human NSCLC cell lines. VCP, also present in FIG. 14A, was co-upregulated with PDL1 (p<0.05, one-tailed z-test). VCP was overexpressed in NSCLC tumor B cells, NSCLC PBL and tumor T cells (FIG. 15), as well as C4./C9-CTLA. CD4 T-cell cluster. VCP was in the 9-gene prognostic marker panel as a survival-hazard gene and was associated with radiotherapy resistance (FIG. 17) in NSCLC patients. Consistently, VCP protein expression was correlated with metastasis and poor prognosis in NSCLC patients. VCP mRNA and/or protein expression was associated with drug sensitivity to cisplatin, etoposide, paclitaxel, vinorelbine, and gemcitabine, and resistance to gefitinib (Tables 8 and 8).

Evidence supports the therapeutic significance of the other genes in the 9-gene prognostic marker panel identified from the B-cell network (FIG. 14A). ANAPC5 was overexpressed in NSCLC tumor B cells and PBL T cells (FIG. 15) and was associated with resistance to cisplatin in NSCLC epithelial cells (Table 8). ANAPC5 protein expression was associated with drug sensitivity to pemetrexed (Table 9). Depletion of ANAPC5 exhibited a synthetic lethal interaction with paclitaxel in NSCLC cells, suggesting enhanced sensitivity to APC/C inhibition in the tumor cells. CCT3, a survival-hazard gene identified in this study, was overexpressed in NSCLC tumor B cells and suppressive tumor Tregs of CD4-C9-CTLA cells vs. other tumor infiltrating Tregs of CD4-C8-FOXP3 cells. CCT3 was under-expressed in NSCLC PBL T cells (FIG. 15). Suppression of CCT3 inhibited tumor progression through the impairment of ATP production and cytoplasmic translation in lung adenocarcinoma. CCT3 mRNA/protein expression was associated with drug sensitivity to erlotinib, etoposide, gefitinib, gemcitabine, pemetrexed, and vinorelbine (Tables 8 and 9). HRH1, an NSCLC survival-hazard gene found in this study, was under-expressed in NSCLC tumor B cells and was associated with resistance to erlotinib and vinorelbine in human NSCLC epithelial cell lines. HRH1 protein expression was associated with sensitivity to pemetrexed (Table 9). HRH1 was reported as an apatinib (VEGFR2 inhibitor) upregulated gene and was also associated with neuroactive ligand in NSCLC cells. HRH1 had different prognostic implications in various cancer types. TUBA1B, an identified survival-hazard gene, was under-expressed in NSCLC tumor B cells. TUBA1B was overexpressed in NSCLC PBL T cells, C14 CD4/CD8 T-cell cluster, and activated tumor Tregs of CD4-C9-CTLA4 (TNFRSF9⁺) vs non-activated tumor Tregs of CD4-C9-CTLA4 (TNFRSF9^{-l ). TUBA}1B mRNA and/or protein expression was associated with resistance to gefitinib, docetaxel, paclitaxel, and sensitivity to etoposide (Tables 8 and 9). TUBA1B protein expression differed significantly between NSCLC patients and healthy individuals and the difference was correlated with the lipid response. These results indicate that the identified network missing in NSCLC tumor B cells (FIG. 14A) has implications for proliferation, prognosis, and therapeutic responses. The pathway enrichment analysis of this network (FIG. 14B) showed over-representation in ribosomal scanning, protein synthesis, and metabolism, consistent with the results from the functional assays of NSCLC cell lines matching the gene expression signature associated with clinical phenotypes (Table 10). These results shed light on dysregulated gene expression networks in singular B cells with prognostic and therapeutic significance in NSCLC.

Among the identified DE genes in NSCLC PBL T cells and tumor T-cell clusters, HLA-DRA, HLA-DRB1, OAS1, and CD74 had concordant prognostic indications at mRNA and protein expression levels in bulk NSCLC tumors with resectable disease. HLA-DRA and HLA-DRB1 belong to HLA class II alpha and beta chain paralogs, respectively. Both HLA-DRA and HLA-DRB1 were under-expressed in NSCLC PBL T cells and overexpressed in NSCLC tumor C11./C6-LAYN. CD8 T-cell cluster, C14 CD4/CD8 T-cell cluster, as well as suppressive tumor Tregs of CD4-C9-CTLA4 cells vs. other tumor infiltrating Tregs of CD4-C8-FOXP3 cells. A higher mRNA and protein expression of HLA-DRA and HLA-DRB1 in bulk NSCLC tumors was associated with prolonged patient survival (FIG. 16). HLA-DRA protein expression was associated with sensitivity to gefitinib and resistance to paclitaxel (Table 9). HLA-DRB1 mRNA expression was associated with resistance to carboplatin (Table 8). HLA-DR protein expression was decreased in tumor infiltrating immune cells and regional lymph nodes of NSCLC. OAS1 was overexpressed in C13 CD4 T-cell cluster./C4-GZMK/C4-CD69 CD8/CD4 T-cell cluster, and suppressive tumor Tregs of CD4-C9CTLA cells vs. other tumor infiltrating Tregs of CD4-C8-FOXP3 cells. OAS1 was under-expressed in NSCLC tumor B cells and PBL T cells (FIG. 15). OAS1 mRNA and protein expression were associated with poor prognosis in NSCLC patients (FIG. 16). OAS1 mRNA/protein expression was associated with resistance to cisplatin and gefitinib, and sensitivity to paclitaxel (Tables 8 and 9). Increased OAS1 expression in cancer cells promotes their ability to survive DNA damage by attenuating Poly(ADP-ribose) synthesis and thus preventing cell death. CD74 was overexpressed in C4./C9-CTLA4. CD4 T-cell cluster, C5 and C6./C4-GZMK. CD8 T-cell clusters, NSCLC tumor B cells, and suppressive tumor Tregs of CD4-C9-CTLA4 cells vs. other tumor infiltrating Tregs of CD4-C8-FOXP3 cells. CD74 was under-expressed in NSCLC PBL T cells (FIG. 15). Higher CD74 mRNA and protein expression was associated with a good prognosis in NSCLC patients (FIG. 16). CD74 protein expression was associated with sensitivity to gemcitabine (Table 9). CD74-NRG1 fusions, originally found in non-smoking lung adenocarcinoma patients, provide the ligand for ERBB2-ERBB3 receptor complexes through the extracellular expression of the EGF-like domain of NRG1 III-β3. CD74-NTRK1 fusions are oncogenic and lead to constitutive TRKA kinase activity in lung cancer.

TIGIT was overexpressed in C4./C9-CTLA. CD4 T-cell cluster, C5 and C6./C4-GZMK. CD8 T-cell clusters, NSCLC tumor B cells, and suppressive tumor Tregs of CD4-C9CTLA cells vs. other tumor infiltrating Tregs of CD4-C8-FOXP3 cells. TIGIT was under-expressed in NSCLC PBL T cells (FIG. 15). TIGIT gene expression was associated with sensitivity to carboplatin and gemcitabine (Table 8). The immunomodulatory receptor TIGIT is an emerging ICI for cancer immunotherapy. Tiragolumab (anti-TIGIT therapy) in combination with atezolizumab (Tecentriq) was approved by the FDA for metastatic NSCLC with high PD-L1 based on promising clinical evidence. TSC22D3 expression induction could lead to the modulation of T cell activation and apoptosis. TSC22D3 was overexpressed in NSCLC tumor B cells and PBL T cells (FIG. 15). TSC22D3 was associated with sensitivity to erlotinib and gefitinib (Tables 8 and 9). TSC22D3 was associated with a good prognosis in NSCLC patients. TSC22D3 was frequently present in the T cell state transitions from intermediate to the pre-dysfunction and dysfunction states. Overall, this study conducted a comprehensive analysis of public single-cell RNA sequencing data and identified DE genes in NSCLC PBL and tumor T cells with prognostic and therapeutic importance.

Targeted therapies and repositioning drugs were identified from the CMap database using a novel Al pipeline integrating Boolean implication modelling of tumor-specific B-cell co-expression networks, DE genes in NSCLC PBL and tumor T cells, bulk tumor transcriptome and proteome, and in vitro CRISPR-Cas9/RNAi and drug screening in human NSCLC epithelial cell lines. The CMap input genes were differentially expressed in single-cell RNA sequencing of NSCLC tumor B cells, PBL and tumor T cells with prognostic and predictive implications in a comprehensive evaluation of public patient data. Their functional involvement in NSCLC cell proliferation was substantiated with public genome-scale CRISPR-Cas9/RNAi screening data. Rigorous filtering criteria were applied in defining the CMap gene expression signature. Three pro-survival genes in the 9-gene prognostic marker panel (ANAPC5, EWSR1, and EXOC4) were removed from the input list due to their proliferative roles in NSCLC indicated by the CRISPR-Cas9/RNAi screening results. Similarly, genes showing any conflicting drug sensitivity or-resistance results to 10 studied therapeutic regimens were removed from the CMap list. As a result, the identified targeted therapies and repositioning drugs are highly relevant to up-regulating epithelial and drug-sensitive genes, down-regulating mesenchymal, drug-resistant, proliferative, and survival-hazard genes in NSCLC patient samples. The selected small molecules were further examined based on their public drug response measurements in 135 human CCLE NSCLC epithelial cell lines. Lesaurtinib, TW-37, and danusertib had small average IC₅₀ and EC₅₀ measurements without any outliers (IC₅₀ or EC₅₀ higher than 10 μM) in 135 cell lines, indicating their efficacy in inhibiting NSCLC cell growth with a potentially safe dose. Danusertib showed an inhibitory association with the gene expression of CD27, an emerging ICI for solid tumors. TG-101348 had an inhibitory association with PDL1 gene expression. Out of the four selected compounds, danusertib, TG-101348 and TW-37 have been investigated in pre-clinical and clinical studies for improving NSCLC treatment. Lestaurtinib, a tyrosine kinase inhibitor of FLT3 for treating AML could potentially be repositioned for treating NSCLC based on its ability to potently kill and inhibit NSCLC cells and its high relevance in reversing EMT, enhancing drug response, inhibiting proliferation in NSCLC cells, and down-regulating survival-hazard genes in NSCLC patients. This Al pipeline can effectively determine the disease relevance of targeted therapies before clinical trials, thereby expediting drug repositioning R&D for pharmaceutical companies. It also provides an efficient tool for oncologists to choose a targeted therapy for refractory NSCLC patients after the failure of initial treatments.

The current single-cell RNA sequencing techniques have not become routine assays for cancer research or clinical testing. Similarly, although CRISPR-Cas9 has been explored in editing immune cells, genome-scale CRISPR-Cas9/RNAi screening data are lacking for broad research applications. This study leveraged public data consortia including TCGA and CCLE. The public single B-cell sequencing data. does not have matched patient clinical follow-up information. This dataset. was used to identify the single B-cell gene co-expression work (FIG. 14) that was missing in tumor B cells. This network consisted of proliferation genes identified from CRISPR-Cas9/RNAi genome-scale screening data of human epithelial NSCLC cell lines. The 9-gene signature was derived from this network showing prognostic capacity in RNA-sequencing profiles of NSCLC bulk tumors. An ideal study design would be using the same patient cohort for single cell sequencing, CRISPR-Cas9/RNAi screening, and prognostication. Nevertheless, it is not technically feasible to conduct such studies currently. The presented conceptual framework could be applied in future research when it is feasible to implement this study design with technology advancement.

There was a remarkable difference in the proportion of low-risk patients in the training and validation sets, i.e., 19% vs. 68%. The training set (GSE81089) utilized snap-frozen tumor samples in RNA-sequencing, whereas the validation set (TCGA). contained mostly formalin-fixed paraffin-embedded (FFPE) samples. The difference in the sample preservation and RNA degradation since fixing is a possible factor contributing to the different RNA expression quantification scales in both datasets. In RNA-sequencing data analysis, housekeeping genes are not generally used for normalization as in qRT-PCR. Therefore, the variation in gene expression measurements due to different sample preparation is not accounted for in the RNA expression analysis. The 9-gene prognostic model was unchanged when applied in the training and validation sets. Therefore, the resulted patient stratification in the validation set containing FFPE samples could be skewed because of the variation in the gene expression measurements caused by different sample preparation. This study showed the feasibility of using this 9-gene signature for patient stratification using RNA-sequencing of bulk tumors. In future clinical applications, the training model based on this 9-gene panel will need to be calibrated according to a specific manufacturing platform to ensure optimal prognostication.

NSCLC remains the leading cause of cancer-related mortality despite the promising results from immunotherapy. There are currently no biomarkers to identify early-stage NSCLC patients of all histology who are at risk for tumor recurrence/metastasis and benefit from adjuvant therapies. There is a pressing demand for predictive biomarkers of immunotherapy in NSCLC. The mechanisms underlying B-cell dysfunction and their prognostic significance in NSCLC are not well understood. To meet these critical needs, this study identified a tumor-specific B-cell proliferation and prognostic gene co-expression network in NSCLC using Boolean implication modelling of single-cell RNA sequencing data. A 9-gene marker panel within this network provided accurate prognostic stratification for early-stage NSCLC patients using RNA sequencing and proteomic profiles. DE genes in NSCLC tumor B cells, PBL and tumor T cells with prognostic implications were garnered. Numerous selected genes had a significant association with radiotherapy response and drug-sensitivity/-resistance to 10 commonly used NSCLC therapeutic regimens. Based on these results, functional pathways, targeted therapies, and repositioning drugs were discovered using a novel Al pipeline to prolong NSCLC patient survival, improve treatment response, inhibit proliferation, and reverse EMT. These rigorous analyses of extensive public data generated solid results and hypotheses for future clinical investigations and will aid the development of novel therapies to improve NSCLC patient outcomes.

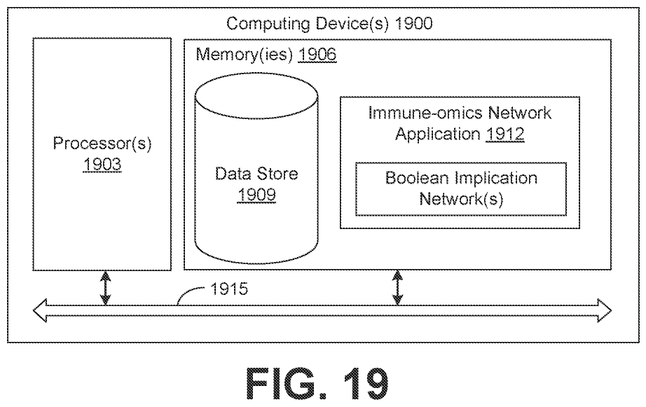

With reference to FIG. 19, shown is a schematic block diagram of a computing device 1900 that can be utilized to execute an immune-omics network application 1912 for biomarker identification and repurposing drug discovery for evaluation and treatment of cancer such as NSCLC. In some embodiments, among others, the computing device 1900 may represent a mobile device (e.g. a smartphone, tablet, laptop computer, etc.). Each computing device 1900 includes at least one processor circuit, for example, having a processor 1903 and a memory 1906, both of which are coupled to a local interface 1915. To this end, each computing device 1900 may comprise, for example, at least one server computer or like device. The local interface 1915 may comprise, for example, a data bus with an accompanying address/control bus or other bus structure as can be appreciated.

Stored in the memory 1906 are both data and several components that are executable by the processor 1903. In particular, stored in the memory 1906 and executable by the processor 1903 are an immune-omics network application 1912 and potentially other applications. Also stored in the memory 1906 may be a data store 1909 and other data. In addition, an operating system may be stored in the memory 1906 and executable by the processor 1903.

It is understood that there may be other applications that are stored in the memory 1906 and are executable by the processor 1903 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

A number of software components are stored in the memory 1906 and are executable by the processor 1903. In this respect, the term “executable” means a program file that is in a form that can ultimately be run by the processor 1903. Examples of executable programs may be, for example, a compiled program that can be translated into machine code in a format that can be loaded into a random access portion of the memory 1906 and run by the processor 1903, source code that may be expressed in proper format such as object code that is capable of being loaded into a random access portion of the memory 1906 and executed by the processor 1903, or source code that may be interpreted by another executable program to generate instructions in a random access portion of the memory 1906 to be executed by the processor 1903, etc. An executable program may be stored in any portion or component of the memory 1906 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1906 is defined herein as including both volatile and nonvolatile memory and data storage components. Volatile components are those that do not retain data values upon loss of power. Nonvolatile components are those that retain data upon a loss of power. Thus, the memory 1906 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1903 may represent multiple processors 1903 and/or multiple processor cores and the memory 1906 may represent multiple memories 1906 that operate in parallel processing circuits, respectively. In such a case, the local interface 1915 may be an appropriate network that facilitates communication between any two of the multiple processors 1903, between any processor 1903 and any of the memories 1906, or between any two of the memories 1906, etc. The local interface 1915 may comprise additional systems designed to coordinate this communication, including, for example, performing load balancing. The processor 1903 may be of electrical or of some other available construction.

Although the immune-omics network application 1912 and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the immune-omics network application 1912, that comprises software or code can be embodied in any non-transitory computer-readable medium for use by or in connection with an instruction execution system such as, for example, a processor 1903 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the immune-omics network application 812, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 800, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0. 1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method for treating non-small cell lung cancer (NSCLC) in a subject, comprising administering to the subject an effective amount of a compound selected from the group consisting of anisomycin, puromycin, aztreonam, ivermectin, natural compound piperlongumine, disulfiram (Antabuse), trichostatin A, dasatinib, lestaurtinib, homosalate, FK 888, penfluridol, and lestaurtinib in a pharmaceutically acceptable carrier.

2. A method for treating non-small cell lung cancer (NSCLC) in a subject, comprising:
(a) assaying a sample from the subject for expression levels of COPA, CSE1L, EIF2AK3, FOSL1, LSM3, MCM5, PMPCB, POLR1B, POLR2F, PSMD11, RPL32, RPS18, SNRPE, SPP1, IFITM3, ATP1B4, PRRX1, FUT7, POF1B, ZNF671, ZBED2, C10orf10, ERH, KLHL6, SERPING1, TRIM29, CCR2, TNFSF12-TNFSF13, ATP1B4, PRDM1, RGS17, CD80, ARK1C1, DEFB103A, CSPP1, AKR1C2, DSG3, PRDM1, ABCA13, ARSE, PTAFR, ZEB1, ERH, F13A1, KLHL6, PRRX1, GPX2, LAIR1, TRIM22, PTAFR, RAB25, ESRP1, SERPINB5, KLHL6, CD80, SH2D1A, CD27, TGM5, CD274, HLA-F, LY75, F13A1, CTSH, CPNE8, ARSE, HLA-DMB, SLC39A8, HNMT, CTSH, CDH3, HNMT, ABCA13, F13A1, DUSP2, HLA-DRB1, TIGIT, TNIP3, ANAPC5, OAS1, VCP, IL2RA, LMNA, PPP2R1A, TNIP3, CCND2, TSC22D3, HRH1, CCT3, EWSR1, TUBA1B, HSD17B13, MAP4, HRH1, CD74, HLA-DRA, HSD17B13, EXOC4, GBP4, TSC22D3, or a combination thereof; and
(b1) detecting elevated levels of DUSP2, VCP, IL2RA, LMNA, CCT3, PPP2R1A, POF1B, CD274, HLA-F, LY75, PRRX1, and/or F13A1, detecting reduced levels of PSMD11, ANAPC5, OAS1, FUT7, ZNF671, ZBED2, C10orf10, CTSH, and/or TRIM29, or a combination thereof, in a sample from the subject, and treating the subject with cisplatin;
(b2) detecting elevated levels of RPS18, PMPCB, TNIP3, IL2RA, CCND2, GBP4, ERH, KLHL6, AKR1C1, AKR1C2, SERPINB5, and/or POLR1B, detecting reduced levels of IFITM3, C10orf10, SERING1, PSMD11, LMNA, PPP2R1A, TUBA1B, CPNE8, and/or EIF2AK3, or a combination thereof, and treating the subject with docetaxel;
(b3) detecting elevated levels of RPL32, TSC22D3, CCT3, VCP, KLHL6, TRIM29, IFITM3, SERPINB5, PRDM1, RGS17, CD80, AKR1C1, AKR1C2, DEFB103A, CSPP1, ARSE, HLA-DMB, TRIM22, POF1B, LSM3, and/or POLR2F, detecting reduced levels of CSE1L, DUSP2, HRH1, LMNA, LY75, TGM5, F13A1, IFITM3, CCR2, TNFSF12-TNFSF13, ATP1B4, SERPING1, DSG3, and/or FOSL1, or a combination thereof, and treating the subject with erlotinib;
(b4) detecting elevated levels of CCT3, EWSR1, TUBA1B, VCP, HSD17B13, LMNA, PSMD11, PRDM1, ATP1B4, SERPING1, ABCA13, HLA-F, ABCA13, and/or SERPINB5, detecting reduced levels of TGM5, TRIM29, and/or ARSE, or a combination thereof, and treating the subject with etoposide;
(b5) detecting elevated levels of CCND2, CCT3, CCT3, HLA-DRA, TSC22D3, CCT3, DEFB103A, PTAFR, CSPP1, KLHL6, TRIM29, and/or SLC39A8, detecting reduced levels of COPA, OAS1, VCP, HSD17B13, LMNA, MAP4, TUBA1B, HRH1, HSD17B13, ZEB1, ERH, F13A1, RGS17, ATP1B4, PRRX1, SERPING1, and/or ARSE, or a combination thereof, and treating the subject with gefitinib;
(b6) detecting elevated levels of CCT3, CD74, DUSP2, HLA-DRA, TIGIT, LMNA, VCP, ARSE, GPX2, RGS17, LAIR1, TRIM22, TGM5, CTSH, HLA-F, SERPINB5, CDH3, HNMT, SPP1, and/or MCM5, detecting reduced levels of PSMC3, EXOC4, SERPING1, ARSE, C10orf10, ABCA13, AKR1C2, GPX2, C10orf10, and/or RPS18, or a combination thereof, and treating the subject with gemcitabine;
(b7) detecting elevated levels of DUSP2, VCP, OAS1, TNIP3, PTAFR, TRIM29, RAB25, ESRP1, SERPINB5, KLHL6, CD80, CD27, CDH3, PRRX1, PMPCB, RPS18, POLR1, and/or LY75, detecting reduced levels of PSMD11, PPP2R1A, HSD17B13, CD74, HLA-DRA, TUBA1B, SH2D1A, C10orf10, HLA-F, SLC39A8, CTSH, and/or TRIM29, or a combination thereof, and treating the subject with paclitaxel;
(b8) detecting elevated levels of CCT3, DUSP2, ANAPC5, HRH1, and/or RPS18, detecting reduced levels of SH2D1A, or a combination thereof, and treating the subject with pemetrexed;
(b9) detecting elevated levels of FUT, VCP, CCT3, DUSP2, CCND2,, SERPINB5, AKR1C2, AKR1C1, F13A1, HLA-F, and/or POLR2F, detecting reduced levels of CSE1L, EXOC4, HRH1, LMNA, TNFSF12-TNFSF13, DSG3, SNRPE, and/or CTSH, or a combination thereof, and treating the subject with vinorelbine; or
(b10) detecting elevated levels of DUSP2, TIGIT, TGM5, and/or IFITM3, detecting reduced levels of HLA-DRB1, TNIP3, ATP1B4, and/or PRRX1, or a combination thereof, and treating the subject with carboplatin.

3. A system, comprising:
a computing device comprising a processor and memory; and
an application to generate Boolean implication networks when compiled with the computing device, the application comprising machine readable instructions stored in the memory that, when executed by the processor, cause the computing device to at least:
generate one or more multi-omics networks from patient cohort information based upon one or more Boolean implication networks;
identify prognostic genes based upon the one or more multi-omics networks and patient gene expression profiles and associated clinical information;
identify proliferation genes based upon the one or more multi-omics networks and screening results in cell lines;
delineating a final multi-omics network based upon identified prognostic and proliferation genes; and
identifying a repurposing drug based upon the final multi-omics network.

4. The system of claim 3, wherein the screening results comprise CRSPR-Cas9 and RNA interference (RNAi).

5. The system of claim 3, wherein the repurposing drug is identified based upon the final multi-omics network using a Connectivity Map database.

6. The system of claim 3, wherein the patient cohort information is associated with cancer, the patient cohort information including disease-free survival time with status and/or disease-specific survival time with status.

7. The system of claim 3, wherein the cancer is non-small cell lung cancer (NSCLC).

8. The system of claim 3, wherein the cell lines are NSCLC.

9. The system of claim 3, comprising obtaining the patient cohort information from a patient cohort database.

10. The system of claim 9, wherein the patient cohort information includes genome-scale mRNA expression profiles and DNA copy number variations from the patient cohort database.

11. The system of claim 9, wherein the patient cohort information is obtained from a NSCLC patient cohort.

12. The system of claim 3, wherein the patient cohort information is evaluated based upon a plurality of implication rules of the one or more Boolean implication networks.

13. The system of claim 3, further comprising identifying genes associated with radiotherapy and chemotherapy.

14-18. (canceled)
